(19) United States
(12) Patent Application Publication
(10) Pub. No.: US 2015/0366890 A1
(43) Pub. Date: Dec. 24, 2015

(54) COMPOSITIONS AND METHODS FOR TREATING FUNGAL INFECTIONS

(71) Applicant: TRUSTEES OF BOSTON UNIVERSITY, Boston, MA (US)

(72) Inventors: James J. COLLINS, Newton, MA (US); Peter A. BELENKY, Framingham, MA (US); Diogo M. CAMACHO, Framingham, MA (US)

(21) Appl. No.: 14/766,592

(22) PCT Filed: Feb. 24, 2014
(86) PCT No.: PCT/US2014/017942

§ 371 Date: Aug. 7, 2015

Related U.S. Application Data
(60) Provisional application No. 61/768,854, filed on Feb. 25, 2013.

(51) Int. Cl. A61K 31/7048; A61K 31/7004; A61K 31/4418; A61K 45/06; A61K 31/4174

(52) U.S. Cl. CPC A61K 31/7048; A61K 45/06; A61K 31/4174; A61K 31/4418; A61K 31/7004

(57) ABSTRACT

The methods and compositions described herein relate to the treatment of fungal infections, e.g. by potentiating the sensitivity of fungi to anti-fungal agents.

### CROSS-REFERENCE TO RELATED APPLICATIONS

This application claims benefit under 35 U.S.C. §119(e) of U.S. Provisional Application No. 61/768,854 filed Feb. 25, 2013, the contents of which are incorporated herein by reference in its entirety.

### GOVERNMENT SUPPORT

This invention was made with Government Support under Grant No. OD003644 awarded by the National Institutes of Health. The Government has certain rights in the invention.

### FIELD OF THE INVENTION

The field of the invention relates to treating and/or potentiating the sensitivity of fungi to antifungal compounds.

### SEQUENCE LISTING

The instant application contains a Sequence Listing which has been submitted electronically in ASCII format and is hereby incorporated by reference in its entirety. Said ASCII copy, created on Feb. 21, 2014, is named 701586-075931-PCT_SL.txt and is 296,876 bytes in size.

### BACKGROUND OF THE INVENTION

A rapid rise in immunocompromised patients over the past five decades has led to increasing incidence of systemic fungal infections. Systemic candidiasis is the most common form of fungal infection in the United States, accounting for approximately 50% of cases, and is the third most common form of bloodstream infection (Ostrosky-Zeichner et al., 2010). Despite current treatment options, the morbidity and mortality rates associated with fungal infections, specifically those of Candida species, remain high. One of the key problems associated with current antifungal treatment is toxicity.

### SUMMARY OF THE INVENTION

As described herein, the inventors have identified a common oxidative damage cellular death pathway triggered by three representative fungicides in Candida albicans and Saccharonmyces cerevisiae. This mechanism utilizes a signaling cascade involving the GTPases Ras1/2 and Protein Kinase A, and culminates in cellular death through the production of toxic hydroxyl radicals in a tricarboxylic acid cycle- and respiratory chain-dependent manner. Consistent with this, cellular mitochondrial activity is substantially elevated by fungicide treatment. In addition, it is demonstrated herein that the metabolome of C. albicans is altered by antifungal drug treatment, exhibiting a shift from fermentation to respiration, a jump in the AMP/ATP ratio, and elevated production of sugars, such as glucose, fructose and trehalose. Additionally. DNA damage is demonstrated herein to play a critical role in antifungal-induced cellular death and blocking DNA repair mechanisms potentiates antifungal activity. Finally, it was found that artificially stimulating the cAMP sensitive RAS pathway with db-cAMP elevated fungicide toxicity.

Accordingly, described herein are methods and compositions relating to the modulation of these pathways to select antifungal agents and/or increase the susceptibility of fungi to antifungal agents. Through metabolic or small molecule based potentiation of currently available antifungal agents, the concentration required to achieve fungal killing can be reduced, thereby alleviating toxicity complications. The invention thereby improves the current arsenal of antifungals.

In one aspect, described herein is a method for inhibiting a fungal infection, the method comprising administering to a subject having or at risk for a fungal infection an effective amount of one or more potentiator compounds and an effective amount of an antifungal agent. In one aspect, described herein is a method for inhibiting a fungal infection, the method comprising administering to a subject having or at risk for a fungal infection an effective amount of a pharmaceutical composition comprising one or more potentiator compounds and an antifungal agent. In one aspect, described herein is a method for treating a fungal infection, comprising administering to a patient having a fungal infection and undergoing treatment with an antifungal agent, an effective amount of one or more potentiator compounds.

In one aspect, described herein is a method for inhibiting fungal growth, the method comprising contacting a fungal cell with an effective amount of one or more potentiator compounds and an effective amount of an antifungal agent.

In one aspect, described herein is a potentiator compound for use in inhibiting or treating a fungal infection, wherein the potentiator compound is an agonist of the RAS/PKA pathway; an agonist of the TCA cycle or respiration; an inhibitor of DNA repair; cAMP or a mimetic or analog thereof; a cAMP modulator; a phosphodiesterase inhibitor, or glucose.

In one aspect, described herein is a composition comprising an antifungal agent formulated in a glucose solution.

In one aspect, described herein is a method comprising selecting a compound that increases ROS production in a target fungal pathogen or that increases ROS-induced cellular damage in the target fungal pathogen, and formulating the compound for treatment of a fungal pathogen, optionally with one or more additional antifungal agents.

In some embodiments of any of the foregoing aspects, the potentiator compound is an agonist of the RAS/PKA pathway; an agonist of the TCA cycle or respiration; an inhibitor of DNA repair, cAMP or a mimetic or analog thereof; a cAMP modulator, a phosphodiesterase inhibitor; or glucose. In some embodiments of any of the foregoing aspects the agonist of the RAS/PKA pathway is an agonist of RAS1; RAS2; Cyr1; Cdc25; Srv2; Tpk1; Tpk2; Tpk3; and orthologs and homologs thereof; or an inhibitor of Bcy1; Pde1; Pde2; or orthologs and homologs thereof. In some embodiments of any of the foregoing aspects, the inhibitor of Pde1 is IC224. In some embodiments of any of the foregoing aspects, the agonist of the TCA cycle or respiration is an agonist of Hap2; Hap3; Hap4; Hap5; Cit1; Cit2; Sdh1/2 or orthologs and homologs thereof. In some embodiments of any of the foregoing aspects, the potentiator compound modulates carbon source utilization or inhibits glucose utilization. In some embodiments of any of the foregoing aspects, the inhibitor of DNA repair is an inhibitor of double-strand break repair; an inhibitor of single-strand repair, or an inhibitor of direct reversal. In some embodiments of any of the foregoing aspects, the inhibitor of double-strand break repair is an inhibitor of Rad54; Rad51; Rad52; Rad55; Rad57; RPA; Xrs2; Mre11; Lif1; Nej1; or orthologs and homologs thereof. In some embodiments of any of the foregoing aspects, the inhibitor is wortmannin; rapamycin; vorinostat; 0⁶-BG; NVP-BEZ235; 2-(Morpholin-4-yl)-benzo[h]chomen-4-one; 1-(2-hydroxy-4-morpholin-4-yl-phenyl)-ethanone; Ku55933; NU7441; or SU11752. In some embodiments of any of the foregoing aspects, the cAMP mimetic or analog or modulator thereof is diburtyryl cAMP; caffeine; forskolin; 8-bromo-cAMP; phorbol ester, sclareline; cholera toxin (CTx); aminophylline; 2,4 dinitrophenol (DNP); norepinephrine; epinephrine; isoproterenol; isobutylmethylxanthine (IBMX); theophylline (dimethylxanthine); dopamine; rolipram; iloprost; prostaglandin E₁; prostaglandin E₂; pituitary adenylate cyclase activating polypeptide (PACAP); vasoactive intestinal polypeptide (VIP); (S)-adenosine; cyclic 3′,5′-(hydrogenphosphorothioate)triethyl ammonium; 8-bromoadenosine-3′,5′-cyclic monophosphate; 8-chloroadenosine-3′,5′-cyclic monophosphate; or N6,2′-O-dibutyryladenosine-3′,5′-cyclic monophosphate. In some embodiments of any of the foregoing aspects, the phosphodiesterase inhibitor is rolipram, mesembrine, drotaverine, roflumilast, ibudilast, piclamilast, luteolin, cilomilast, diazepam, arofylline, CP-80633, denbutylline, drotaverine, etazolate, filaminast, glaucine, HT-0712, ICI-63197, irsogladine, mesembrine, Ro20-1724, RPL-554, YM-976, sildenafil, vardenafil, tadalafil, udenafil, avanafil sofyllin, pentoxifylline, acetildenafil, bucladesine, cilostamide, cilostazol, dipyridamole, enoximone, glaucine, ibudilast, icariin, inamrinone (formerly amrinone), lodenafil, luteolin, milrinone, mirodenafil, pimobendan, propentofylline, zardaverine, caffeine, theophylline, theobromine, 3-isobutyl-1-methylxanthine (IBMX), aminophylline, or paraxanthine. In some embodiments of any of the foregoing aspects, the potentiator is selected for its ability to increase ROS production or increase susceptibility to oxidative stress. In some embodiments of any of the foregoing aspects, the ROS is O₂⁻, H₂O₂, or O₂⁻ and H₂O₂.

In some embodiments of any of the foregoing aspects, the antifungal is fungicidal or fungistatic. In some embodiments of any of the foregoing aspects, the antifungal agent is a polyene; an imidazole; a triazole; a thiazole; an allylamine; or an echinocandin; or any salts or variants thereof. In some embodiments of any of the foregoing aspects, the polyene antifungal agent is amphotericin B; candicidin; filipin; hamycin; natamycin; nystatin; or rimocidin. In some embodiments of any of the foregoing aspects, the imidazole antifungal agent is bifonazole; butoconazole; clotrimazole; econazole; fenticonzole; isoconazole; ketoconazole; miconazole; omoconazole; oxiconazole; sertaconazole; sulconazole; or tioconazole. In some embodiments of any of the foregoing aspects, the trizaole antifungal agent is albaconazole; fluconazole; isavuconazole; itraconazole; posaconazole; ravuconazole; terconazole; or voriconazole. In some embodiments of any of the foregoing aspects, the thiazole antifunal agent is abafungin. In some embodiments of any of the foregoing aspects, the allylamine antifungal agent is amorolfin; butenafine; naftifine; or terbinafine. In some embodiments of any of the foregoing aspects, the echinocandin is anidulafungin; caspofungin; or micafungin. In some embodiments of any of the foregoing aspects, the antifungal agent is benzoic acid; ciclopirox; flucytosine; griseofulvin; haloprogin; polygodial; tolnaftate; undecylenic acid; or crystal violet.

In some embodiments of any of the foregoing aspects, the fungal infection is an infection of skin or soft tissue; a superficial mycosis; a cutaneous mycosis; a subcutaneous mycosis; a vaginal mycosis; a systemic mycosis; or is an infected wound or burn. In some embodiments of any of the foregoing aspects, the infection is a surface wound, burn, or infection; infection of a mucosal surface; respiratory infection; infections of the eyes, ears, nose, or throat; or infection of an intestinal pathogen. In some embodiments of any of the foregoing aspects, the fungal infection is resistant to one or more anti-fungal agents. In some embodiments of any of the foregoing aspects, the fungal infection involves one or more of: Candida spp.; Cryptococcus spp.; Aspergillus spp.; Microsporum spp.; Trichophyton spp.; Epidermophyton spp.; Trichosporon spp.; Fusarium spp.; Tinea versicolor; Tinea barbae; Tinea corporis; Tinea cruris; Tinea manuum; Tinea pedis; Tinea unguium; Tinea faciei; Tinea imbricate; Tinea incognito; Epidermophyton floccosum; Microsporum canis; Microsporum audouinii; Trichophyton interdigitale; Trichophyton mentagrophytes; Trichophyton tonsurans; Trichophyton schoenleini; Trichophyton rubrum; Hortaea werneckii; Piedraia hortae; Malasserzia furfur; Coccidioides immitis; Coccidioidesposadasii; Histoplasma capsulatum; Histoplasma duboisii; Lacazia loboi; Paracoccidioides brasiliensis; Blastomyces dermatitidis; Sporothrix schenckii; Penicillium marneffei; Candida albicans; Candida glabrata; Candida tropicalis; Candida lusitaniae; Candida jirovecii; Candida krusei; Candida parapsilosi; Exophiala jeanselmei; Fonsecaea pedrosoi; Fonsecasea compacta; Phialophora verrucosa; Geotrichum candidum; Pseudallescheria boydii; Rhizopus oryzae; Muco indicus; Absidia corymbifera; Synceplasastrum racemosum; Basidiobolus ranarum; Conidiobolus coronatus; Conidiobolus incongruous; Cryptococcus neoformans; Enterocytozoan bieneusi; Encephalitozoon intestinalis; and Rhinosporidium seeberi.

In some embodiments of any of the foregoing aspects, the potentiator compound and the antifungal agent are co-formulated. In some embodiments of any of the foregoing aspects, the potentiator compound is glucose. In some embodiments of any of the foregoing aspects, the potentiator compound and the antifungal agent are administered separately. In some embodiments of any of the foregoing aspects, the potentiator compound is administered systemically or locally. In some embodiments of any of the foregoing aspects, the potentiator compound is administered intravenously, orally, or topically. In some embodiments of any of the foregoing aspects, the fungal infection occurs at or in a surface wound or burn, and the potentiator compound is administered topically to the affected area. In some embodiments of any of the foregoing aspects, the potentiator compound is formulated as a cream, gel, foam, spray, or as a tablet or capsule for oral delivery.

### BRIEF DESCRIPTION OF THE DRAWINGS

FIGS. 1A-1E demonstrate that fungicide-Dependent ROS Production Leads to Fungal Cell Death and a Common Transcriptional Response. FIGS. 1A-1B depict graphs of the generation of ROS as measured by a change in HPF fluorescence after 1.5 hours of drug treatment in S. cerevisiae (FIG. 1A) and C. albicans (FIG. 1B). FIG. 1C depicts a graph of Log of CFU/ml remaining after drug exposure in the presence and absence of 50 mM thiourea (TU). Drug concentrations used: AMB 1 μg/ml, MCZ 50 μg/ml, CIC 75 μg/ml, H₂O₂ 1 mM, geneticin (G418) 100 μg/ml, fluconazol (FLC) 50 μg/ml, 5-flucytosine (5-FC) 15 μg/ml, and ketoconazole (KTZ) 501 μg/ml. The reported error is standard deviation (s.d.) with n≧3. FIG. 1D depicts a schematic of the common transcriptional response to antifungal treatment identified by performing differential expression analysis against a compendium of expression arrays. The common set of differentially expressed genes is represented by the intersection of the three sets of genes. FIG. 1E depicts a schematic of pathway analysis of the common set of differentially expressed genes, which identified six major processes that are upregulated in response to antifungals and three processes that are downregulated under the same treatments.

FIGS. 2A-2F demonstrate that TCA-Dependent Respiration and the Ras/PKA Pathway Play a Critical Role in Antifungal-Induced Cell Death. FIGS. 2A and 2E depict graphs of Log of CFU/ml remaining after three hours of drug exposure of wildtype S. cerevisiae and mutants targeting the TCA cycle, respiration and the Ras/PKA pathway. FIGS. 2B and 2F depict graphs of cellular ROS levels quantified as percent change in fluorescence after the addition of HPF. The indicated strains were treated with antifungal drugs for 1 hour prior to the addition of HPF. Drug concentrations used: AMB 1 μg/ml, MCZ 50 μg/ml, and CIC 75 μg/ml. The reported error is s.d. with n≧3. FIG. 2C depicts a graph of Log of CFU/ml remaining after three hours of drug exposure. Drug concentrations used, increasing from left to right: AMB (1 μg/ml, 1.5 μg/ml, 2.5 μg/ml and 8 μg/ml), MCZ (50 μg/ml, 75 μg/ml, 100 μg/ml and 150 μg/ml), and CIC (75 μg/ml 100 μg/ml, 125 μg/ml and 150 μg/ml). FIG. 2D depicts a graph of yeast mitochondrial content assayed by fluorescent staining with MitoTracker Red probe after one hour of exposure to the indicated drug or metabolite.

FIGS. 3A-3E demonstrate that antifungal treatment leads to common metabolic changes resulting in the production of sugars and a dramatic reduction of ATP levels. FIG. 3A depicts a schematic of the metabolomics study. Cells were treated with antifungal drugs for 1.5 hours and intracellular metabolites were analyzed using mass spectrometry to identify the AF-perturbed metabolome. FIG. 3B depicts a graph of fold change in metabolite levels compared to the no-treatment control. FIGS. 3C-3D depict graphs of relative signal intensity of select metabolites identified through metabolomic profiling of C. albicans exposed to antifungal drugs for 1.5 hours. Drug concentrations used: AMB 1 μg/ml, MCZ 50 μg/ml, and CIC 75 μg/ml. The reported error is standard error mean with n=6. FIG. 3E depicts a graph of AMP/ATP ratio in C. albicans treated with drugs over a 90 min period. Drug concentrations used: AMB 0.35 μg/ml, MCZ 50 μg/ml, and CIC 75 μg/ml. The reported error is s.d. with an n≧3.

FIGS. 4A-4I demonstrate that DNA repair is a critical response to antifungal-dependent ROS production. FIGS. 4A-4C depict graphs of Log of CFU/ml remaining after 3 hours of drug exposure of wildtype C. albicans and mutants targeting double-strand break repair (DSBR (rad50/rad50 and rad52/rad52)), nucleotide excision (NER(rad10/rad10)) and mismatch (MMR (msh1/msh1)) DNA repair mechanisms. FIG. 4D depicts a graph of TUNEL staining of exponentially growing cells assayed by flow cytometry, after 2 hours of treatment. Drug concentrations used, decreasing from left to right: H₂O₂ (10 mM and 5 mM), AMB (1 μg/ml, and 0.5 μg/ml), MCZ (100 μg/ml, and 50 μg/ml), and CIC (150 μg/ml and 75 μg/ml). FIGS. 4E-4H depicts graphs of Log of CFU/ml remaining after 3 hours of drug exposure of wildtype S. cerevisiae and mutants targeting DSBR. The reported error is s.d. with n≧3. FIG. 4I depicts a schematic of the proposed common mechanism of AF action for the tested fungicides: antifungal activity against primary intracellular targets leads to cellular changes sensed by the RAS/PKA signaling pathway. The RAS/PKA signaling cascade induces mitochondrial activity, leading to the production of ROS. Simultaneously, the production of sugars, consistent with the fungal stress response, leads to the rapid consumption of ATP and the production of AMP. Elevated intracellular ROS production leads to cellular death through damage to DNA and other cellular targets.

FIGS. 5A-5C demonstrate the generation of hydroxyl radicals measured by a change in 3′-(p-hydroxyphenyl) fluorescein (HPF) fluorescence after 1.5 hours of drug exposure. FIGS. 5A-5B depict graphs of the cell count of S. cerevisiae exposed to cidal and static drugs, respectively. FIG. 5C depicts a graph of cell counts of C. albicans exposed to cidal drugs. Chromatographs of treated cells without HPF incubation are included to demonstrate the HPF-dependent change in fluorescence for each treatment. Drug concentrations used: AMB 1 μg/ml, MCZ 50 μg/ml, CIC 75 μg/ml, H₂O₂ 1 mM, geneticin (G418) 100 μg/ml, fluconazol (FLC) 50 μg/ml, 5-flucytosine (5-FC) 15 μg/ml, and ketoconazole (KTZ) 50 μg/ml.

FIGS. 6A-6F depict graphs of Log of CFU/ml remaining after drug exposure. Drug concentrations used: AMB 1 pig/ml, MCZ 50 μg/ml, and CIC 75 μg/ml. The reported error is standard deviation (s.d.) with n≧3.

FIGS. 7A-7D depict graphs of kill curves of metabolically profiled C. albicans cultures. Log of CFU/ml remaining after exposure of wildtype C. albicans to the indicated drug concentrations. Cells were collected at the time point indicated by the red oval and sent for metabolomic profiling. The letter and number combinations refer to specific samples that were metabolically profiled and are consistent with the labels in the supplemental data set.

FIGS. 8A-8D demonstrate that caspofungin-induced metabolic changes match the changes predicted by the common mechanism. FIG. 8A depicts a graph of the fold change in metabolite levels compared to the no-treatment control. FIGS. 8B-8C depict graphs of the relative signal intensity of select metabolites identified through metabolomic profiling of C. albicans exposed to antifungal drugs for 1.5 hours. Drug concentrations used: AMB 1 μg/ml, MCZ 50 μg/ml, CIC 75 μg/ml and caspofungin (CAS) 0.5 μg/ml. The reported error is standard error mean with n=6. FIG. 8D depicts a graph of the generation of hydroxyl radicals measured by a change in 3′-(p-hydroxyphenyl) fluorescein (HPF) fluorescence after 1.5 hours of exposure to CAS at 0.5 μg/ml. Caspofungin acetate was provided by Merck Research Laboratories (Rahway, N.J.).

FIG. 9 demonstrates that cAMP modulators elevate antifungal activity. Graphs of Log of CFU/ml remaining after exposure of wildtype C. albicans to AMB at the indicated drug concentrations are depicted. Cells were pretreated with db-cAMP or caffeine for 30 minutes prior to the addition of AMB.

FIGS. 10A-10D demonstrate that trehalose pathway activity modulates antifungal activity. FIGS. 10A-10C depict graphs of cell growth in wild-type and tps1 and tps2 mutants following treatment with amphotericin B, miconazole, and ciclopirox respectively. FIG. 10D depicts a graph of the production of hydroxyl radicals in wild-type and tsp1 and tsp2 mutants following treatment with the indicated antifungal agents. HPF=3′-(p-hydroxyphenyl) fluorescein.

FIGS. 11A-11F demonstrate the effect of glucose concentrations on the activity of antifungal agents. FIG. 11A depicts a graph of C. albicans growth after treated with the indicated antifungal agents in the presence of the indicated levels of glucose. FIG. 11B depicts a graph of C. albicans growth after treated with the indicated antifungal agents in the presence of the indicated levels of glucose. Log of CFU/ml after 3 hours of drug exposure of C. albicans cultured in SDC media with the indicated glucose concentrations. Log of CFU/ml before treatment was 6.5±0.3. Drug concentrations used: AMB 0.3 μg/ml, MCZ 25 μg/ml, CIC 65 μg/ml. The reported error is the s.d. with n 3. FIG. 11C depicts a graph of growth curves of C. albicans incubated at the indicated glucose concentration (mg/ml). FIGS. 11D-F depict graphs of the log of CFU/ml remaining after drug exposure of C. albicans incubated at the indicated glucose concentration (mg/ml). The reported error is the s.d. with n?3.

### DETAILED DESCRIPTION

Provided herein are compositions and methods comprising potentiator compounds that, e.g. increase ROS production and/or enhance ROS-induced cellular damage, thereby potentiating oxidative attack by antifungal agents. The potentiator targets were identified, in part, using the systems-based, genome-scale ROS metabolic models and experimental validation, as described herein. The compositions, methods, and approaches described herein provide efficient means of improving treatment of fungal infections and inhibiting fungal replication and growth, by, for example, increasing efficacy and potency of antifungal agents, including known agents such as amphotericin, miconzole, and ciclopirox. By increasing efficacy and potency of known antifungal agents, the compositions and methods comprising potentiator compounds also permit lower dosages of antifungal agents to be used with increased efficacy.

As described herein, a systems biology approach was used to identify a common oxidative damage cellular death pathway triggered by three representative fungicides in Candida albicans and Saccharomynces cerevisiae. This mechanism utilizes a signaling cascade involving the GTPases Ras1/2 and Protein Kinase A, and culminates in cellular death through the production of toxic hydroxyl radicals in a tricarboxylic acid cycle- and respiratory chain-dependent manner. In addition, it is demonstrated herein that the metabolome of C. albicans is altered by antifungal drug treatment, exhibiting a shift from fermentation to respiration, a jump in the AMP/ATP ratio, and elevated production of sugars, such as glucose, fructose and trehalose. Based on this data a model is proposed herein in which antifungals (AFs) activate a common signaling and metabolic cascade that leads to ROS-dependent cellular death. This model can be used to select antifungal compounds or potentiator compounds for treatment of fungal infections.

While some of these pathways have been implicated in the fungal response to environmental stresses, e.g. acid exposure, methods of potentiating the action of antifungal agents by modulating the pathways and targets as described herein has not been previously described. For instance, US Patent Publication No. 20120093817 described inhibiting, e.g. RAS1 and RAS2, in order to treat fungal infections. This is directly opposed to the methods described herein, which relate to, e.g. increasing the activity of, e.g. RAS1 and RAS2, to potentiate the activity of antifungal agents.

Furthermore, the methods and compositions described herein represent the application of the particularly surprising discovery that certain pathways which have been implicated in the potentiating of antibacterial agents can be similarly utilized in treating fungal infections. The finding of such a similarity between eurkaryotes and prokaryotes; which are notably disparate organisms with quite different metabolic pathways; particularly in the context of the action of entirely divergent antimicrobial compounds (e.g. antifungal agent are structurally and functionally unrelated to antibacterials, e.g. exemplary antifungal agents target ergosterol or ergosterol biosynthesis) is unexpected.

Accordingly, provided herein in some aspects, are potentiation targets and inhibitors and/or agonists of such targets, termed herein as “potentiator compounds” that impact, e.g. ROS production and/or ROS-induced damage repair mechanisms (e.g. DNA damage repair mechanisms), and compositions and methods of their use thereof.

### Potentiator Compositions and Methods Thereof

As described herein, using the systems-based, genome-scale ROS metabolic models described herein and experimental validation, novel biochemical targets have been identified that potentiate oxidative attack by antifungals and biocide by increasing ROS flux and endogenous ROS production as well as increasing susceptibility to intracellular damage created by ROS. Accordingly, provided herein, in some aspects, are compositions, including therapeutic compositions and combinations, comprising an effective amount of a potentiator compound, and methods of preventing or treating fungal infection with the same.

The term “potentiator compound,” as used herein, refers to an agent or compound that increases production of reactive oxygen species (ROS) in fungal pathogens (e.g. increasing basal ROS production in cells), or in addition or alternatively by inhibiting cellular ROS-damage repair mechanisms. By increasing endogenous ROS production in fungal cells, as described herein, the inventors have discovered that this increases or potentiates the activity or action of fungicides and increases sensitivity to oxidants, and consequent killing of fungi. Additionally, in some embodiments, a potentiator compound can inhibit ROS-induced cellular damage, e.g. inhibit double-strand break (DSB) repair. Accordingly, a potentiator can, in some embodiments, be considered an adjuvant of an antifungal agent for which it acts to potentiate its activity. Thus, in some aspects, provided herein are therapeutic compositions comprising a potentiator compound and an antifungal agent.

A potentiator compound or agent described herein can increase or stimulate the sensitivity of a fungal cell to an antifungal agent by about at least 20% or more, at least 30% or more, at least 40% or more, at least 50% or more, at least 60% or more, at least 70% or more, at least 80% or more, at least 90% or more, at least 95% or more, at least 100%, at least 2-fold greater, at least 5-fold greater, at least 10-fold greater, at least 25-fold greater, at least 50-fold greater, at least 100-fold greater, at least 1000-fold greater, and all amounts in-between, in comparison to a reference or control level of sensitivity in the absence of the potentiator compound, or in the presence of the antifungal alone. Methods and assays to identify such potentiator compounds can be based on any method known to one of skill in the art, are found throughout the specification, in the drawings, and in the Examples section.

As used herein, the term “adjuvant” can also be used to refer to an agent, such as the potentiator compounds described herein, which enhances or potentiates the pharmaceutical effect of another agent, such as an antifungal agent, e.g., a polyene or azole antifungal agent. In some embodiments, e.g., the potentiator compounds, as disclosed herein, function as adjuvants to those antifungal agents that cause or act, in part, via ROS production, by further increasing basal ROS production in a cell or inhibiting ROS-induced damage repair mechanisms, and thereby potentiating the activity of the antifungal agents by about at least 20% or more, at least 30% or more, at least 40% or more, at least 50% or more, at least 60% or more, at least 70% or more, at least 80% or more, at least 90% or more, at least 95% or more, at least 100%, at least 2-fold greater, at least 5-fold greater, at least 10-fold greater, at least 25-fold greater, at least 50-fold greater, at least 100-fold greater, at least 1000-fold greater, and all amounts in-between, as compared to use of the antifungal agent alone.

The term “agent” as used herein in reference to a potentiator compound means any compound or substance such as, but not limited to, a small molecule, nucleic acid, polypeptide, peptide, drug, ion, etc. An “agent” can be any chemical, entity, or moiety, including, without limitation, synthetic and naturally-occurring proteinaceous and non-proteinaceous entities. In some embodiments of the aspects described herein, an agent is a nucleic acid, a nucleic acid analogue, a protein, an antibody, a peptide, an aptamer, an oligomer of nucleic acids, an amino acid, or a carbohydrate, and includes, without limitation, ribozymes, DNAzymes, glycoproteins, antisense RNAs, siRNAs, lipoproteins, and modifications and combinations thereof etc. Compounds for use in the therapeutic compositions and methods described herein can be known to have a desired activity and/or property, e.g., increase endogenous ROS production or increase ROS-induced cellular damage, or can be selected from a library of diverse compounds, using screening methods known to one of ordinary skill in the art.

As used herein, the term “small molecule” refers to a chemical agent an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole. Such small molecules can take the form of salts, esters, and other pharmaceutically acceptable forms of such compounds.

The following classes of modulators, as exemplified in C. albicans, can be effective for increasing antifungal senstivity in fungi, including, but not limited to, C. albicans, according to the compositions and methods described herein. Such fungi include others with similar metabolic systems and other species determined to have similar metabolic systems using the systems-based, genome-scale ROS metabolic models described herein and consequent experimental validation.

In some aspects, the invention provides a method for making an antifungal composition. The method comprises selecting a compound that increases ROS production in a target fungal pathogen, and/or that increases ROS-induced cellular damage in the target fungal pathogen, and formulating the compound for treatment of a fungal pathogen, optionally with one or more additional antifungal agents. In some embodiments, the compound increases ROS production by modulating fungal respiration. For example, the compound may increase TCA cycle or electron transport chain activity (e.g., while reducing fermentation or sugar usage), and/or may render the TCA cycle or electron transport chain activity less efficient, to thereby increase endogenous ROS production. The level of endogenous ROS production can be determined by any suitable assay including assays disclosed herein. In some embodiments, the compound activates the RAS/PKA pathway to thereby promote endogenous ROS production. In these or other embodiments, the compound increases ROS-induced cellular damage, for example, by inhibiting cellular repair mechanisms. Such cellular repair mechanisms include DNA repair mechanisms, which may be assayed by any suitable technique including those described herein. Compounds may be selected from those described herein or from any suitable library of compounds.

In some embodiments, a potentiator compound can be selected from the following: an agonist of the RAS/PKA pathway; an agonist of the TCA cycle or respiration; an inhibitor of DNA repair, cAMP or a mimetic or analog thereof; a cAMP modulator, a phosphodiesterase inhibitor; or glucose. In some embodiments, the potentiator compound can increase ROS production or increase susceptibility to oxidative stress. In some embodiments, the ROS can be O₂⁻, H₂O₂, or O₂⁻ and H₂O₂. In some embodiments, the potentiator compound can inhibit DSB repair. Assays and methods for determining ROS production and/or DSB repair are known to one of ordinary skill in the art and are described in the Examples herein.

As used herein an “agonist of the RAS/PKA pathway” refers to an agent which can increase the activity of the RAS/PKA pathway by at least 20% or more, 30% or more, 50% or more, 100% or more, 200% or more, or greater. Increased activity of the RAS/PKA pathway can be determined, e.g. by detecting increased level of phosphorylation of Thr197 or Thr198 of PKA, increased levels of cytosolic cAMP, and/or increased levels of phosphorylation of targets of PKA (e.g., trehalase, Atg1, Msn2, Msn4, Rim15, Pde2, and/or Pde1). Methods of detecting phosphorylated proteins are known in the art, e.g. the use of antibody reagents specific for particular phosphorylated peptides. Such reagents are commercially available (e.g. anti phospho-PKA (Cat. No. sc-32968 Santa Cruz Biotechnology; Dallas, Tex.). In some embodiments, an agonist of the RAS/PKA pathway can be an agonist of an enzyme selected from RAS1 (e.g., NCBI Ref Seq: XP_{—}714365 (SEQ ID NO: 1)); RAS2 (e.g., NCBI Ref Seq: XP_{—}722969 (SEQ ID NO: 2)); Cyr1 (e.g., NCBI Ref Seq: XP_{—}716904 (SEQ ID NO: 3)); Cdc25 (e.g., NCBI Ref Seq: XP_{—}711071 (SEQ ID NO: 4)); Srv2 (e.g., NCBI Ref Seq: XP_{—}717368 (SEQ ID NO: 5)); Tpk1 (e.g., NCBI Ref Seq: NP_{—}012371 (SEQ ID NO: 6)); Tpk2 (e.g., NCBI Ref Seq: XP_{—}714866 (SEQ ID NO: 7)); Tpk3 (e.g., NCBI Ref Seq: XP_{—}723386 (SEQ ID NO: 8)); and orthologs and homologs thereof; or an inhibitor of an enzyme selected from: Bcy1 (e.g., NCBI Ref Seq: XP_{—}719591 (SEQ ID NO: 9)); Pde1 (e.g., NCBI Ref Seq: XP_{—}720545 (SEQ ID NO: 10)); Pde2 (e.g. NCBI Ref Seq: XP_{—}721471 (SEQ ID NO: 11)) and orthologs and homologs thereof. In some embodiments, an inhibitor of PdI can be IC224.

In some embodiments, the potentiator compound is cAMP, or a mimietic or analog of modulator thereof, e.g. the potentiator compound can cause a change in the cell that mimics that caused by exogenous and/or increased levels of cAMP, e.g. the potentiator compound can increase the level of activity of the RAS/PKA pathway. Non-limiting examples of cAMP mimetics, analogs, or modulators thereof can include diburtyryl cAMP; caffeine; forskolin; 8-bromo-cAMP; phorbol ester, sclareline; cholera toxin (CTx); aminophylline; 2,4 dinitrophenol (DNP); norepinephrine; epinephrine; isoproterenol; isobutylmethylxanthine (IBMX); theophylline (dimethylxanthine); dopamine; rolipram; iloprost; prostaglandin Et; prostaglandin E₂; pituitary adenylate cyclase activating polypeptide (PACAP); vasoactive intestinal polypeptide (VIP); (S)-adenosine; cyclic 3′,5′-(hydrogenphosphorothioate)triethyl ammonium; 8-bromoadenosine-3′,5′-cyclic monophosphate; 8-chloroadenosine-3′,5′-cyclic monophosphate; and N6,2′-O-dibutyryladenosine-3′,5′-cyclic monophosphate.

In some embodiments, the potentiator compound can be a phosphodiesterase inhibitor. Non-limiting examples of phosphodiesterase inhibitors can include rolipram, mesembrine, drotaverine, roflumilast, ibudilast, piclamilast, luteolin, cilomilast, diazepam, arofylline, CP-80633, denbutylline, drotaverine, etazolate, filaminast, glaucine, HT-0712, ICI-63197, irsogladine, mesembrine, Ro20-1724, RPL-554, YM-976, sildenafil, vardenafil, tadalafil, udenafil, avanafil, sofyllin, pentoxifylline, acetildenafil, bucladesine, cilostamide, cilostazol, dipyridamole, enoximone, glaucine, ibudilast, icariin, inamrinone (formerly amrinone), lodenafil, luteolin, milrinone, mirodenafil, pimobendan, propentofylline, zardaverine, caffeine, theophylline, theobromine, 3-isobutyl-1-methylxanthine (IBMX), aminophylline, and paraxanthine.

As used herein an “agonist of the TCA cycle or respiration” refers to an agent which can increase the activity of the TCA cycle or respriation by at least 20% or more, 30% or more, 50% or more, 100% or more, 200% or more, or greater. The activity of the TCA cycle or respiration pathway can be determined, e.g. by detecting an increase in CO₂ and/or a decrease in water, acetate. Methods of detecting the levels of CO₂, water, and/or acetate are well known in the art, e.g. HPLC or enzymatic assays (see, e.g. Cat. No. BQ 007-EAEL from Gentaur, Brussels, Belgium). In some embodiments, the agonist of the TCA cycle or respiration is an agonist of an enzyme selected from: Hap2 (e.g., NCBI Ref Seq:XP_{—}716482 (SEQ ID NO: 12)); Hap3 (e.g., NCBI Ref Seq:XP_{—}717380 (SEQ ID NO: 13)); Hap4 (e.g., NCBI Ref Seq:NP_{—}012813 (SEQ ID NO: 14)); Hap5 (e.g., NCBI Ref Seq:XP_{—}715679 (SEQ ID NO: 15)); Cit1 (e.g., NCBI Ref Seq:XP_{—}715118 (SEQ ID NO: 16)); Cit2 (e.g., NCBI Ref Seq: NP_{—}009931 (SEQ ID NO: 17)); Sdh1/2 (e.g. NCBI Ref Seqs: XP_{—}715875 (SEQ ID NO: 18) and XP_{—}712003 (SEQ ID NO: 19)) and orthologs and homologs thereof.

As used herein, an “inhibitor of DNA repair” refers to an agent that can reduce the level of DNA repair by at least 20%, at least 30%, at least 40%, at least 50% or more. The level of DNA repair can be detected, e.g. by determining the level of single strand breaks (e.g. using the comet assay as described in Collins. Mol Biotechnol. 2004 26:249-61; which is incorporated by reference herein in its entirety and/or FLARE™ assays (e.g., Cat No. 4130-100-FK; Amsbio; Abington, UK)) or as described below herein for the detection of DSB. In some embodiments, the inhibitor of DNA repair can be an inhibitor of double-strand break repair; an inhibitor of single-strand repair; or an inhibitor of direct reversal. In some embodiments, an inhibitor of direct reversal can be, e.g. an inhibitor of Mgt1 (e.g., NCBI Ref Seq: NP_{—}010081 (SEQ ID NO: 20)) or Phr1 (e.g., NCBI Ref Seq: NP_{—}015031 (SEQ ID NO: 21)) or orthologs and homologs thereof, e.g. an inhibitor of an enzyme that chemically reverses damage to a DNA base. In some embodiments, an inhibitor of single-strand repair can be an inhibitor of Ogg1 (e.g. NCBI Ref Seq: NP_{—}013651 (SEQ ID NO: 22)), Mag1 (e.g. NCBI Ref Seq: NP_{—}011069 (SEQ ID NO: 23)), Ung1 (e.g., NCBI Ref Seq: NP_{—}013691 (SEQ ID NO: 24)), Apn1 (e.g. NCBI Ref Seq: NP_{—}012808 (SEQ ID NO: 25)), Apn2 (e.g. NCBI Ref Seq: NP_{—}009534 (SEQ ID NO: 26)), Tpp1 (e.g., NCBI Ref Seq: NP_{—}013877 (SEQ ID NO: 27)), Rad27 (e.g. NCBI Ref Seq: NP_{—}012809 (SEQ ID NO: 28)), Cdc9 (e.g. NCBI Ref Seq: NP_{—}010117 (SEQ ID NO: 29)), Ccl1 (e.g. NCBI Ref Seq: NP_{—}015350 (SEQ ID NO: 30)), Kin28 (e.g. NCBI Ref Seq: NP_{—}010175 (SEQ ID NO: 31)), Rad10 (e.g. NCBI Ref Seq: NP_{—}013614 (SEQ ID NO: 32)), Rad2 (e.g. NCBI Ref Seq: NP_{—}011774 (SEQ ID NO: 33)), Rad25 (e.g. NCBI Ref Seq: NP_{—}012123 (SEQ ID NO: 34)), Rad1 (e.g. NCBI Ref Seq: NP_{—}015303 (SEQ ID NO: 35)), Rad26 (e.g. NCBI Ref Seq: NP_{—}012569 (SEQ ID NO: 36)), Rad28 (e.g. NCBI Ref Seq: NP_{—}010313 (SEQ ID NO: 37)), Tfb3 (e.g. NCBI Ref Seq: NP_{—}010748 (SEQ ID NO: 38)), Met18 (e.g. NCBI Ref Seq: NP_{—}012138 (SEQ ID NO: 39)), Rfa1 (e.g. NCBI Ref Seq: NP_{—}009404 (SEQ ID NO: 40)), Rfa2 (e.g. NCBI Ref Seq: NP_{—}014087 (SEQ ID NO: 41)), Syf1 (e.g. NCBI Ref Seq: NP_{—}010704 (SEQ ID NO: 42)), Rad14 (e.g. NCBI Ref Seq: NP_{—}013928 (SEQ ID NO: 43)), Rad4 (e.g. NCBI Ref Seq: NP_{—}011089 (SEQ ID NO: 44)), Msh2 (e.g. NCBI Ref Seq: NP_{—}014551 (SEQ ID NO: 45)), Msh6 (e.g. NCBI Ref Seq: NP_{—}010382 (SEQ ID NO: 46)), Msh3 (e.g. NCBI Ref Seq: NP_{—}010016 (SEQ ID NO: 47)), Mlh1 (e.g. NCBI Ref Seq: NP_{—}013890 (SEQ ID NO: 48)), Pms1 (e.g. NCBI Ref Seq: NP_{—}014317 (SEQ ID NO: 49)) and orthologs or homologs thereof; e.g. an agent that reduces the level of mismatches and/or single base damage (e.g. oxidation, alkylation, hydrolysis, or deamination).

As used herein, an “inhibitor of double-strand break repair” refers to an agent that can reduce the level of DSB repair by at least 20%, at least 30%, at least 40%, at least 50% or more. The level of DSB repair can be detected, e.g. by determining the amount of double strand breaks present in a cell, e.g. by a TUNEL assay as described in the Examples herein. In some embodiments, the inhibitor of double-strand break reapir is an inhibitor of inhibitor of Rad54 (e.g., NCBI Ref Seq:XP_{—}722208 (SEQ ID NO: 50)); Rad51(e.g., NCBI Ref Seq: XP_{—}713440 (SEQ ID NO: 51)); Rad52 (e.g., NCBI Ref Seq: XP_{—}711260 (SEQ ID NO: 52)); Rad55 (e.g., NCBI Ref Seq: NP_{—}010361 (SEQ ID NO: 53)); Rad57 (e.g., NCBI Ref Seq: XP_{—}715957 (SEQ ID NO: 54)); RPA (e.g., NCBI Ref Seq: XP_{—}719539 (SEQ ID NO: 55)); Xrs2 (e.g., NCBI Ref Seq: NP_{—}010657 (SEQ ID NO: 56)); Mre11 (e.g., NCBI Ref Seq: XP_{—}712486 (SEQ ID NO: 57)); Lif1 (e.g., NCBI Ref Seq: NP_{—}011425 (SEQ ID NO: 58)); Nej1 (e.g., NCBI Ref Seq: NP_{—}013367 (SEQ ID NO: 59)); and orthologs and homologs thereof. Non-limiting examples of inhibitors of double-strand break repair can include wortmannin; rapamycin; vorinostat; 0⁶-BG; NVP-BEZ235; 2-(Morpholin-4-yl)-benzo[h]chomen-4-one; 1-(2-hydroxy-4-morpholin-4-yl-phenyl)-ethanone; Ku55933; NU7441; and SUI 1752.

In some embodiments, the potentiator compound modulates carbon source utilization and/or inhibits glucose utilization. In some embodiments, the potentitator compound can be glucose. In some embodiments, a potentiator compound can be another sugar upregulated by the presence of antifungal agents, e.g. fructose, mannose, and/or trehalose. In some embodiments, the sugar, e.g. glucose is provided at a concentration of at least 0.1%, e.g. 0.1% or greater, 0.5% or greater, 1% or greater, or 2% or greater. In some embodiments, the sugar, e.g. glucose is provided at a concentration of at least 1.0%. In some embodiments, the sugar, e.g. glucose is provided at a concentration of at least 2.0% In some embodiments, the sugar, e.g. glucose is provided at a concentration and dose sufficient to raise blood glucose levels to at least 1.5 mg/mL, e.g. 1.5 mg/mL or greater, 2.0 mg/mL or greater, 2.5 mg/mL or greater, or 3.0 mg/mL or greater. In some embodiments, the sugar, e.g. glucose is provided at a concentration and dose sufficient to raise blood glucose levels to at least 1.75 mg/mL. In some embodiments, the sugar, e.g. glucose is provided at a concentration and dose sufficient to raise blood glucose levels to at least 2.0 mg/mL. In some embodiments, the sugar, e.g. glucose is provided at a concentration and dose sufficient to raise blood glucose levels to at least 2.25 mg/mL. In some embodiments, the sugar, e.g. glucose is provided at a concentration and dose sufficient to raise blood glucose levels to at least 2.5 mg/mL.

In some embodiments, the potentiator can be a modulator of iron metabolism and/or homeostatsis (e.g. FET3 (e.g. NCBI Ref Seq: NP_{—}013774 (SEQ ID NO: 60)) or homologs or orthologs thereof). In some embodiments, the potentiator can be a modulator of free iron accumulation in the mitochondria and/or regulator of Fe—S cluster protein synthesis in the mitochondira.

Agonists or activators of a given target (e.g. an enzyme and/or gene encoding an enzyme that is described as a target of a potentiator compound above herein) can include agents that bind to a target, and stimulates, increases or upregulates expression of, or enhances enzymatic activity of the target. An increase in target activity or expression is achieved by an activator when the activity of or expression of a target polypeptide or a polynucleotide encoding the target is at least 20% higher, at least 30% higher, at least 40% higher, at least 50% higher, at least 60% higher, at least 70% higher, at least 80% higher, at least 90%, at least 100% higher, at least 2-fold higher, at least 3-fold higher, at least 5-fold higher, at least 10-fold higher, at least 15-fold higher, at least 25-fold higher, at least 50-fold higher, at least 100-fold higher, at least 1000-fold higher, or more, relative to a reference activity or expression of a target polypeptide or polynucleotide encoding the target in the absence of the activator. In some embodiments of these aspects, the activator or agonist is an antibody or antigen-binding fragment thereof, a polypeptide, a small molecule, or an activating nucleic acid molecule, such as an activating RNA molecule.

An activating nucleic acid molecule can be a nucleic acid molecule encoding the target polypeptide. Such activating nucleic acid molecules can be comprised by a vector and/or operably linked to control sequences (e.g. a promoter), e.g. in the manner described for inhibitory nucleic acids elsewhere herein.

When antibodies or antigen-binding fragments thereof are used in activating target activity and/or expression, it is understood that the antibody or antigen-binding fragment thereof is an “activating” antibody or an antibody “agonist,” i.e., it is one that increases or promotes biological activity of the target upon binding. For example, an activating antibody can bind a target and promote or increase the ability of the target to, e.g. phosphorylate a substrate or bind to a nucleic acid sequence. Accordingly, in some embodiments of these aspects, the target activating antibody or antigen-binding fragment thereof is an antibody fragment. Activating antibodies or antigen-binding fragments thereof can take any of the forms for antibodies or antigen-binding fragments thereof described herein in the context of antagonist or inhibitory antibodies.

As used herein, the term “inhibitor” refers to an agent which can decrease the expression and/or activity of a target expression product (e.g. mRNA encoding a target or a target polypeptide), e.g. by at least 20% or more, 50% or more, 70% or more, 80% or more, 90% or more, 95% or more, or 98% or more. The efficacy of an inhibitor, e.g. its ability to decrease the level and/or activity of a particular target can be determined, e.g. by measuring the level of an expression product of the target and/or the activity of target. Methods for measuring the level of a given mRNA and/or polypeptide are known to one of skill in the art, e.g. RTPCR with primers can be used to determine the level of RNA and Western blotting with an antibody specific for the target polypetpide can be used to determine the level of the target polypeptide. The activity of the targets described herein can be determined using methods known in the art and described in the Examples herein, including, by way of non-limiting example, by measuring DSB repair using a TUNEL assay to determine if an agent is an inhibitor of DSB repair.

In some embodiments, inhibitors of a particular target gene can be an inhibitory antibody reagent, e.g. an antibody or antigen-binding antibody fragment that binds to and inhibits the activity of the target polypeptide. Methods of making antibodies are known in the art and described elsewhere herein. When antibodies or antigen-binding fragments thereof are used in inhibiting the activity and/or expression of a target, it is understood that the antibody or antigen-binding fragment thereof is a “blocking” antibody or an antibody “antagonist,” i.e., it is one that inhibits or reduces biological activity of the target upon binding, and does not activate or promote the activity of the target. For example, an antagonist antibody can bind a target and inhibit the ability of the target to, for example, phosphorylate a substrate or bind to a DNA sequence. In certain embodiments, the blocking antibodies or antagonist antibodies or fragments thereof described herein completely inhibit the biological activity of the target.

In some embodiments, inhibitors of the expression of a given gene can be an inhibitory nucleic acid. In some embodiments, the inhibitory nucleic acid is an inhibitory RNA (iRNA). Double-stranded RNA molecules (dsRNA) have been shown to block gene expression in a highly conserved regulatory mechanism known as RNA interference (RNAi). The inhibitory nucleic acids described herein can include an RNA strand (the antisense strand) having a region which is 30 nucleotides or less in length, i.e., 15-30 nucleotides in length, generally 19-24 nucleotides in length, which region is substantially complementary to at least part of an mRNA transcript of a target. The use of these iRNAs enables the targeted degradation of mRNA transcripts of the target, resulting in decreased expression and/or activity of the target. The following detailed description discloses how to make and use compositions containing iRNAs to inhibit the expression of a particular target.

As used herein, the term “iRNA” refers to an agent that contains RNA as that term is defined herein, and which mediates the targeted cleavage of an RNA transcript via an RNA-induced silencing complex (RISC) pathway. In one embodiment, an iRNA as described herein effects inhibition of the expression and/or activity of a target described herein.

In one aspect, an RNA interference agent includes a single stranded RNA that interacts with a target RNA sequence to direct the cleavage of the target RNA. Without wishing to be bound by theory, long double stranded RNA introduced into plants and invertebrate cells is broken down into siRNA by a Type III endonuclease known as Dicer (Sharp et al., Genes Dev. 2001, 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleaves the target to induce silencing (Elbashir, et al., (2001) Genes Dev. 15:188). Thus, in one aspect, an RNA interference agent relates to a double stranded RNA that promotes the formation of a RISC complex comprising a single strand of RNA that guides the complex for cleavage at the target region of a target transcript to effect silencing of the target gene.

In some embodiments, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of a target gene. The other strand (the sense strand) includes a region that is complementary to the antisense strand, such that the two strands hybridize and form a duplex structure when combined under suitable conditions. Generally, the duplex structure is between 15 and 30 inclusive, more generally between 18 and 25 inclusive, yet more generally between 19 and 24 inclusive, and most generally between 19 and 21 base pairs in length, inclusive. Similarly, the region of complementarity to the target sequence is between 15 and 30 inclusive, more generally between 18 and 25 inclusive, yet more generally between 19 and 24 inclusive, and most generally between 19 and 21 nucleotides in length, inclusive. In some embodiments, the dsRNA is between 15 and 20 nucleotides in length, inclusive, and in other embodiments, the dsRNA is between 25 and 30 nucleotides in length, inclusive. As the ordinarily skilled person will recognize, the targeted region of an RNA targeted for cleavage will most often be part of a larger RNA molecule, often an mRNA molecule. Where relevant, a “part” of an mRNA target is a contiguous sequence of an mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 15 nucleotides in length, preferably 15-30 nucleotides in length.

One of skill in the art will also recognize that the duplex region is a primary functional portion of a dsRNA, e.g., a duplex region of 9 to 36, e.g., 15-30 base pairs. Thus, in one embodiment, to the extent that it becomes processed to a functional duplex of e.g., 15-30 base pairs that targets a desired RNA for cleavage, an RNA molecule or complex of RNA molecules having a duplex region greater than 30 base pairs is a dsRNA. Thus, an ordinarily skilled artisan will recognize that in one embodiment, then, an miRNA is a dsRNA. In another embodiment, a dsRNA is not a naturally occurring miRNA. In another embodiment, an iRNA agent useful to target Theml expression is not generated in the target cell by cleavage of a larger dsRNA.

While a target sequence is generally 15-30 nucleotides in length, there is wide variation in the suitability of particular sequences in this range for directing cleavage of any given target RNA. Various software packages and the guidelines set out herein provide guidance for the identification of optimal target sequences for any given gene target, but an empirical approach can also be taken in which a “window” or “mask” of a given size (as a non-limiting example, 21 nucleotides) is literally or figuratively (including, e.g., in silico) placed on the target RNA sequence to identify sequences in the size range that may serve as target sequences. By moving the sequence “window” progressively one nucleotide upstream or downstream of an initial target sequence location, the next potential target sequence can be identified, until the complete set of possible sequences is identified for any given target size selected. This process, coupled with systematic synthesis and testing of the identified sequences (using assays as described herein or as known in the art) to identify those sequences that perform optimally can identify those RNA sequences that, when targeted with an iRNA agent, mediate the best inhibition of target gene expression.

A dsRNA as described herein can further include one or more single-stranded nucleotide overhangs. The dsRNA can be synthesized by standard methods known in the art as further discussed below, e.g., by use of an automated DNA synthesizer, such as are commercially available from, for example, Biosearch, Applied Biosystems, Inc. In one embodiment, the antisense strand of a dsRNA has a 1-10 nucleotide overhang at the 3′ end and/or the 5′ end. In one embodiment, the sense strand of a dsRNA has a 1-10 nucleotide overhang at the 3′ end and/or the 5′ end. In one embodiment, at least one end of a dsRNA has a single-stranded nucleotide overhang of 1 to 4, generally 1 or 2 nucleotides. dsRNAs having at least one nucleotide overhang have unexpectedly superior inhibitory properties relative to their blunt-ended counterparts.

In another embodiment, one or more of the nucleotides in the overhang is replaced with a nucleoside thiophosphate. As used herein, the term “nucleotide overhang” refers to at least one unpaired nucleotide that protrudes from the duplex structure of an iRNA, e.g., a dsRNA. For example, when a 3′-end of one strand of a dsRNA extends beyond the 5′-end of the other strand, or vice versa, there is a nucleotide overhang. A dsRNA can comprise an overhang of at least one nucleotide; alternatively the overhang can comprise at least two nucleotides, at least three nucleotides, at least four nucleotides, at least five nucleotides or more. A nucleotide overhang can comprise or consist of a nucleotide/nucleoside analog, including a deoxynucleotide/nucleoside. The overhang(s) may be on the sense strand, the antisense strand or any combination thereof. Furthermore, the nucleotide(s) of an overhang can be present on the 5′ end, 3′ end or both ends of either an antisense or sense strand of a dsRNA.

The terms “blunt” or “blunt ended” as used herein in reference to a dsRNA mean that there are no unpaired nucleotides or nucleotide analogs at a given terminal end of a dsRNA, i.e., no nucleotide overhang. One or both ends of a dsRNA can be blunt. Where both ends of a dsRNA are blunt, the dsRNA is said to be blunt ended. To be clear, a “blunt ended” dsRNA is a dsRNA that is blunt at both ends, i.e., no nucleotide overhang at either end of the molecule. Most often such a molecule will be double-stranded over its entire length.

In some embodiments, a dsRNA will include two oligonucleotides, where one oligonucleotide is described as the sense strand and the second oligonucleotide is described as the corresponding antisense strand of the sense strand. As described elsewhere herein and as known in the art, the complementary sequences of a dsRNA can also be contained as self-complementary regions of a single nucleic acid molecule, as opposed to being on separate oligonucleotides.

The skilled person is well aware that dsRNAs having a duplex structure of between 20 and 23, but specifically 21, base pairs have been hailed as particularly effective in inducing RNA interference (Elbashir et al., EMBO 2001, 20:6877-6888). However, others have found that shorter or longer RNA duplex structures can be effective as well. In some embodiments, dsRNAs described herein can include at least one strand of a length of minimally 21 nt. Hence, dsRNAs having a partial sequence of at least 15, 16, 17, 18, 19, 20, or more contiguous nucleotides, and differing in their ability to inhibit the expression of a target by not more than 5, 10, 15, 20, 25, or 30% inhibition from a dsRNA comprising the full sequence, are contemplated according to the invention.

Further, it is contemplated, further optimization could be achieved by systematically either adding or removing nucleotides to generate longer or shorter sequences and testing those and sequences generated by walking a window of the longer or shorter size up or down the target RNA from that point. Again, coupling this approach to generating new candidate targets with testing for effectiveness of iRNAs based on those target sequences in an inhibition assay as known in the art or as described herein can lead to further improvements in the efficiency of inhibition. Further still, such optimized sequences can be adjusted by, e.g., the introduction of modified nucleotides as described herein or as known in the art, addition or changes in overhang, or other modifications as known in the art and/or discussed herein to further optimize the molecule (e.g., increasing serum stability or circulating half-life, increasing thermal stability, enhancing transmembrane delivery, targeting to a particular location or cell type, increasing interaction with silencing pathway enzymes, increasing release from endosomes, etc.) as an expression inhibitor.

An iRNA as described herein can contain one or more mismatches to the target sequence. In one embodiment, an iRNA as described herein contains no more than 3 mismatches. If the antisense strand of the iRNA contains mismatches to a target sequence, it is preferable that the area of mismatch not be located in the center of the region of complementarity. If the antisense strand of the iRNA contains mismatches to the target sequence, it is preferable that the mismatch be restricted to be within the last 5 nucleotides from either the 5′ or 3′ end of the region of complementarity. For example, for a 23 nucleotide iRNA agent RNA strand which is complementary to a region of a target, the RNA strand generally does not contain any mismatch within the central 13 nucleotides. The methods described herein or methods known in the art can be used to determine whether an iRNA containing a mismatch to a target sequence is effective in inhibiting the expression of the target. Consideration of the efficacy of iRNAs with mismatches in inhibiting expression of a target is important, especially if the particular region of complementarity in the target mRNA is known to have polymorphic sequence variation within the population.

In yet another embodiment, the RNA of an iRNA, e.g., a dsRNA, is chemically modified to enhance stability or other beneficial characteristics. The nucleic acids featured in the invention may be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of RNA compounds useful in the embodiments described herein include, but are not limited to RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In particular embodiments, the modified RNA will have a phosphorus atom in its internucleoside backbone.

Modified RNA backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and US Pat. RE39464, each of which is herein incorporated by reference

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, each of which is herein incorporated by reference.

In other RNA mimetics suitable or contemplated for use in iRNAs, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, each of which is herein incorporated by reference. Further teaching of PNA compounds can be found, for example, in Nielsen et al., Science, 1991, 254, 1497-1500.

Some embodiments featured in the invention include RNAs with phosphorothioate backbones and oligonucleosides with heteroatom backbones, and in particular —CH₂—NH—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —N(CH₃)—CH₂—CH₂— [wherein the native phosphodiester backbone is represented as —O—P—O—CH₂—] of the above-referenced U.S. Pat. No. 5,489,677, and the amide backbones of the above-referenced U.S. Pat. No. 5,602,240. In some embodiments, the RNAs featured herein have morpholino backbone structures of the above-referenced U.S. Pat. No. 5,034,506.

Modified RNAs can also contain one or more substituted sugar moieties. The iRNAs, e.g., dsRNAs, featured herein can include one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Exemplary suitable modifications include O[(CH₂)ₙO]ₘCH₃, O(CH₂).ₙOCH₃, O(CH₂)ₙNH₂, O(CH₂)ₙCH₃, O(CH₂)ₙONH₂, and O(CH₂)ₙON[(CH₂)CH₃)]₂, where n and m are from 1 to about 10. In other embodiments, dsRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an iRNA, or a group for improving the pharmacodynamic properties of an iRNA, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂, also described in examples herein below.

Other modifications include 2′-methoxy (2′-OCH₃), 2′-aminopropoxy(2′-OCH₂CH₂CH₂NH₂) and 2′-fluoro (2′-F). Similar modifications can also be made at other positions on the RNA of an iRNA, particularly the 3′ position of the sugar on the 3′ terminal nucleotide or in 2′-5′ linked dsRNAs and the 5′ position of 5′ terminal nucleotide. iRNAs may also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application, and each of which is herein incorporated by reference.

An iRNA can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., Angewandte Chemie, International Edition, 1991, 30, 613, and those disclosed by Sanghvi, Y S., Chapter 15, dsRNA Research and Applications, pages 289-302, Crooke, S. T. and Lebleu, B., Ed., CRC Press, 1993. Certain of these nucleobases are particularly useful for increasing the binding affinity of the oligomeric compounds featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 6,015,886; 6,147,200; 6,166,197; 6,222,025; 6,235,887; 6,380,368; 6,528,640; 6,639,062; 6,617,438; 7,045,610; 7,427,672; and 7,495,088, each of which is herein incorporated by reference, and U.S. Pat. No. 5,750,692, also herein incorporated by reference.

The RNA of an iRNA can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193).

Representative U.S. patents that teach the preparation of locked nucleic acid nucleotides include, but are not limited to, the following: U.S. Pat. Nos. 6,268,490; 6,670,461; 6,794,499; 6,998,484; 7,053,207; 7,084,125; and 7,399,845, each of which is herein incorporated by reference in its entirety.

Another modification of the RNA of an iRNA featured in the invention involves chemically linking to the RNA one or more ligands, moieties or conjugates that enhance the activity, cellular distribution, pharmacokinetic properties, or cellular uptake of the iRNA. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

In one embodiment, a ligand alters the distribution, targeting or lifetime of an iRNA agent into which it is incorporated. In preferred embodiments a ligand provides an enhanced affinity for a selected target, e.g, molecule, cell or cell type, compartment, e.g., a fungal cell, as, e.g., compared to a species absent such a ligand. Preferred ligands will not take part in duplex pairing in a duplexed nucleic acid.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin or hyaluronic acid); or a lipid. The ligand may also be a recombinant or synthetic molecule, such as a synthetic polymer, e.g., a synthetic polyamino acid. Examples of polyamino acids include polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a funal cell, e.g., a lectin, glycoprotein, lipid or protein, e.g., an antibody, that binds to a specified cell type such as a fungal cell, among others. Non-limiting examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a fungal cell. They can also include non-peptidic species, such as lipids, lectins, carbohydrates, vitamins, cofactors, multivalent lactose, multivalent galactose, N-acetyl-galactosamine, N-acetyl-gulucosamine multivalent mannose, or multivalent fucose.

The ligand can be a substance, e.g, a drug, which can increase the uptake of the iRNA agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

In some embodiments, a ligand attached to an iRNA as described herein acts as a pharmacokinetic (PK) modulator. As used herein, a “PK modulator” refers to a pharmacokinetic modulator. PK modulators include lipophiles, bile acids, steroids, phospholipid analogues, peptides, protein binding agents, PEG, vitamins etc. Examplary PK modulators include, but are not limited to, cholesterol, fatty acids, cholic acid, lithocholic acid, dialkylglycerides, diacylglyceride, phospholipids, sphingolipids, naproxen, ibuprofen, vitamin E, biotin etc. Oligonucleotides that comprise a number of phosphorothioate linkages are also known to bind to serum protein, thus short oligonucleotides, e.g., oligonucleotides of about 5 bases, 10 bases, 15 bases or 20 bases, comprising multiple of phosphorothioate linkages in the backbaone are also amenable to the present invention as ligands (e.g. as PK modulating ligands). In addition, aptamers that bind serum components (e.g. serum proteins) are also suitable for use as PK modulating ligands in the embodiments described herein.

For macromolecular drugs and hydrophilic drug molecules, which cannot easily cross bilayer membranes, entrapment in endosomal/lysosomal compartments of the cell is thought to be the biggest hurdle for effective delivery to their site of action. A number of approaches and strategies have been devised to address this problem. For liposomal formulations, the use of fusogenic lipids in the formulation have been the most common approach (Singh, R. S., Goncalves, C. et al. (2004). On the Gene Delivery Efficacies of pH-Sensitive Cationic Lipids via Endosomal Protonation. A Chemical Biology Investigation. Chem. Biol. 11, 713-723.). Other components, which exhibit pH-sensitive endosomolytic activity through protonation and/or pH-induced conformational changes, include charged polymers and peptides. Examples may be found in Hoffman, A. S., Stayton, P. S. et al. (2002). Design of “smart” polymers that can direct intracellular drug delivery. Polymers Adv. Technol. 13, 992-999; Kakudo, Chaki, T., S. et al. (2004). Transferrin-Modified Liposomes Equipped with a pH-Sensitive Fusogenic Peptide: An Artificial Viral-like Delivery System. Biochemistry 436, 5618-5628; Yessine, M. A. and Leroux, J. C. (2004). Membrane-destabilizing polyanions: interaction with lipid bilayers and endosomal escape of biomacromolecules. Adv. Drug Deliv. Rev. 56, 999-1021; Oliveira, S., van Rooy, I. et al. (2007). Fusogenic peptides enhance endosomal escape improving iRNA-induced silencing of oncogenes. Int. J. Pharm. 331, 211-4. They have generally been used in the context of drug delivery systems, such as liposomes or lipoplexes. For folate receptor-mediated delivery using liposomal formulations, for instance, a pH-sensitive fusogenic peptide has been incorporated into the liposomes and shown to enhance the activity through improving the unloading of drug during the uptake process (Turk, M. J., Reddy, J. A. et al. (2002). Characterization of a novel pH-sensitive peptide that enhances drug release from folate-targeted liposomes at endosomal pHs is described in Biochim. Biophys. Acta 1559, 56-68).

In certain embodiments, the endosomolytic components can be polyanionic peptides or peptidomimetics which show pH-dependent membrane activity and/or fusogenicity. A peptidomimetic can be a small protein-like chain designed to mimic a peptide. A peptidomimetic can arise from modification of an existing peptide in order to alter the molecule's properties, or the synthesis of a peptide-like molecule using unnatural amino acids or their analogs. In certain embodiments, they have improved stability and/or biological activity when compared to a peptide. In certain embodiments, the endosomolytic component assumes its active conformation at endosomal pH (e.g., pH 5-6). The “active” conformation is that conformation in which the endosomolytic component promotes lysis of the endosome and/or transport of the modular composition of the invention, or its any of its components (e.g., a nucleic acid), from the endosome to the cytoplasm of the cell.

Libraries of compounds can be screened for their differential membrane activity at endosomal pH versus neutral pH using a hemolysis assay. Promising candidates isolated by this method may be used as components of the modular iRNA delivery compositions. A method for identifying an endosomolytic component for use in the compositions and methods described herein may comprise: providing a library of compounds; contacting blood cells with the members of the library, wherein the pH of the medium in which the contact occurs is controlled; determining whether the compounds induce differential lysis of blood cells at a low pH (e.g., about pH 5-6) versus neutral pH (e.g., about pH 7-8).

Exemplary endosomolytic components include the GALA peptide (Subbarao et al., Biochemistry, 1987, 26: 2964-2972), the EALA peptide (Vogel et al., J. Am. Chem. Soc., 1996, 118: 1581-1586) (“EALA” is disclosed as SEQ ID NO: 61), and their derivatives (Turk et al., Biochem. Biophys. Acta, 2002, 1559: 56-68). In certain embodiments, the endosomolytic component can contain a chemical group (e.g., an amino acid) which will undergo a change in charge or protonation in response to a change in pH. The endosomolytic component may be linear or branched. Exemplary primary sequences of endosomolytic components include H2N-(AALEALAEALEALAEALEALAEAAAAGGC)-CO2H (SEQ ID NO: 62); H2N-(AALAEALAEALAEALAEALAEALAAAAGGC)-CO2H (SEQ ID NO: 63); and H2N-(ALEALAEALEALAEA)-CONH2 (SEQ ID NO: 64).

In certain embodiments, more than one endosomolytic component can be incorporated into the iRNA agent of the invention. In some embodiments, this will entail incorporating more than one of the same endosomolytic component into the iRNA agent. In other embodiments, this will entail incorporating two or more different endosomolytic components into iRNA agent.

These endosomolytic components can mediate endosomal escape by, for example, changing conformation at endosomal pH. In certain embodiments, the endosomolytic components can exist in a random coil conformation at neutral pH and rearrange to an amphipathic helix at endosomal pH. As a consequence of this conformational transition, these peptides may insert into the lipid membrane of the endosome, causing leakage of the endosomal contents into the cytoplasm. Because the conformational transition is pH-dependent, the endosomolytic components can display little or no fusogenic activity while circulating in the blood (pH ˜7.4). “Fusogenic activity,” as used herein, is defined as that activity which results in disruption of a lipid membrane by the endosomolytic component. One example of fusogenic activity is the disruption of the endosomal membrane by the endosomolytic component, leading to endosomal lysis or leakage and transport of one or more components of the modular composition of the invention (e.g., the nucleic acid) from the endosome into the cytoplasm.

In addition to hemolysis assays, as described herein, suitable endosomolytic components can be tested and identified by a skilled artisan using other methods. For example, the ability of a compound to respond to, e.g., change charge depending on, the pH environment can be tested by routine methods, e.g., in a cellular assay. In certain embodiments, a test compound is combined with or contacted with a cell, and the cell is allowed to internalize the test compound, e.g., by endocytosis. An endosome preparation can then be made from the contacted cells and the endosome preparation compared to an endosome preparation from control cells. A change, e.g., a decrease, in the endosome fraction from the contacted cell vs. the control cell indicates that the test compound can function as a fusogenic agent. Alternatively, the contacted cell and control cell can be evaluated, e.g., by microscopy, e.g., by light or electron microscopy, to determine a difference in the endosome population in the cells. The test compound and/or the endosomes can labeled, e.g., to quantify endosomal leakage.

In another type of assay, an iRNA agent described herein is constructed using one or more test or putative fusogenic agents. The iRNA agent can be labeled for easy visulization. The ability of the endosomolytic component to promote endosomal escape, once the iRNA agent is taken up by the cell, can be evaluated, e.g., by preparation of an endosome preparation, or by microscopy techniques, which enable visualization of the labeled iRNA agent in the cytoplasm of the cell. In certain other embodiments, the inhibition of gene expression, or any other physiological parameter, may be used as a surrogate marker for endosomal escape.

In other embodiments, circular dichroism spectroscopy can be used to identify compounds that exhibit a pH-dependent structural transition. A two-step assay can also be performed, wherein a first assay evaluates the ability of a test compound alone to respond to changes in pH, and a second assay evaluates the ability of a modular composition that includes the test compound to respond to changes in pH.

In another aspect, the ligand is a cell-permeation agent, preferably a helical cell-permeation agent. Preferably, such agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent is preferably an alpha-helical agent, which preferably has a lipophilic and a lipophobic phase.

Peptides suitable for use with the present invention can be a natural peptide, e.g., tat or antennopedia peptide, a synthetic peptide, or a peptidomimetic. Furthermore, the peptide can be a modified peptide, for example peptide can comprise non-peptide or pseudo-peptide linkages, and D-amino acids. A peptidomimetic (also referred to herein as an oligopeptidomimetic) is a molecule capable of folding into a defined three-dimensional structure similar to a natural peptide. The attachment of peptide and peptidomimetics to iRNA agents can affect pharmacokinetic distribution of the iRNA, such as by enhancing cellular recognition and absorption. The peptide or peptidomimetic moiety can be about 5-50 amino acids long, e.g., about 5, 10, 15, 20, 25, 30, 35, 40, 45, or 50 amino acids long.

A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP (SEQ ID NO: 65). An RFGF analogue (e.g., amino acid sequence AALLPVLLAAP (SEQ ID NO: 66)) containing a hydrophobic MTS can also be a targeting moiety. The peptide moiety can be a “delivery” peptide, which can carry large polar molecules including peptides, oligonucleotides, and protein across cell membranes. For example, sequences from the HIV Tat protein (GRKKRRQRRRPPQ (SEQ ID NO: 67)) and the Drosophila Antennapedia protein (RQIKIWFQNRRMKWKK (SEQ ID NO: 68)) have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature, 354:82-84, 1991). Preferably the peptide or peptidomimetic tethered to a dsRNA agent via an incorporated monomer unit is a cell targeting peptide such as an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

A “cell permeation peptide” is capable of permeating a cell, e.g., a microbial cell, such as a bacterial or fungal cell, or a mammalian cell, such as a human cell. A microbial cell-permeating peptide can be, for example, an α-helical linear peptide (e.g., LL-37 or Ceropin P1), a disulfide bond-containing peptide (e.g., α-defensin, β-defensin or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al., Nucl. Acids Res. 31:2717-2724, 2003).

In some embodiments, the iRNA oligonucleotides described herein further comprise carbohydrate conjugates. The carbohydrate conjugates are advantageous for the in vivo delivery of nucleic acids, as well as compositions suitable for in vivo therapeutic use, as described herein. As used herein, “carbohydrate” refers to a compound which is either a carbohydrate per se made up of one or more monosaccharide units having at least 6 carbon atoms (which may be linear, branched or cyclic) with an oxygen, nitrogen or sulfur atom bonded to each carbon atom; or a compound having as a part thereof a carbohydrate moiety made up of one or more monosaccharide units each having at least six carbon atoms (which may be linear, branched or cyclic), with an oxygen, nitrogen or sulfur atom bonded to each carbon atom. Representative carbohydrates include the sugars (mono-, di-, tri- and oligosaccharides containing from about 4-9 monosaccharide units), and polysaccharides such as starches, glycogen, cellulose and polysaccharide gums. Specific monosaccharides include C₅ and above (preferably C₅-C₈) sugars; di- and trisaccharides include sugars having two or three monosaccharide units (preferably C5-C₈). In some embodiments, the carbohydrate conjugate further comprises other ligand such as, but not limited to, PK modulator, endosomolytic ligand, and cell permeation peptide.

In some embodiments, the conjugates described herein can be attached to the iRNA oligonucleotide with various linkers that can be cleavable or non cleavable. The term “linker” or “linking group” means an organic moiety that connects two parts of a compound. Linkers typically comprise a direct bond or an atom such as oxygen or sulfur, a unit such as NR⁸, C(O), C(O)NH, SO, SO₂, SO₂NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O, S, S(O), SO₂, N(R⁸), C(O), substituted or unsubstituted aryl, substituted or unsubstituted heteroaryl, substituted or unsubstituted heterocyclic; where R⁸ is hydrogen, acyl, aliphatic or substituted aliphatic. In one embodiment, the linker is between 1-24 atoms, preferably 4-24 atoms, preferably 6-18 atoms, more preferably 8-18 atoms, and most preferably 8-16 atoms.

A cleavable linking group is one which is sufficiently stable outside the cell, but which upon entry into a target cell is cleaved to release the two parts the linker is holding together. In a preferred embodiment, the cleavable linking group is cleaved at least 10 times or more, preferably at least 100 times faster in the target cell or under a first reference condition (which can, e.g., be selected to mimic or represent intracellular conditions) than in the blood or a non-fungal cell environment of a subject, or under a second reference condition (which can, e.g., be selected to mimic or represent conditions found in, e.g. the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower, enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing the cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. Further examples of cleavable linking groups include but are not limited to, redox-cleavable linking groups (e.g. a disulphide linking group (—S—S—)), phosphate-based cleavable linkage groups, ester-based cleavable linking groups, and peptide-based cleavable linking groups. Representative U.S. patents that teach the preparation of RNA conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941; 6,294,664; 6,320,017; 6,576,752; 6,783,931; 6,900,297; 7,037,646; each of which is herein incorporated by reference.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In preferred embodiments, useful candidate compounds are cleaved at least 2, 4, 10 or 100 times faster in the cell (e.g. a fungal cell) (or under in vitro conditions selected to mimic intracellular conditions) as compared to blood or serum (or under in vitro conditions selected to mimic extracellular conditions).

It is not necessary for all positions in a given compound to be uniformly modified, and in fact more than one of the aforementioned modifications can be incorporated in a single compound or even at a single nucleoside within an iRNA. The present invention also includes iRNA compounds that are chimeric compounds. “Chimeric” iRNA compounds or “chimeras,” in the context of this invention, are iRNA compounds, preferably dsRNAs, which contain two or more chemically distinct regions, each made up of at least one monomer unit, i.e., a nucleotide in the case of a dsRNA compound. These iRNAs typically contain at least one region wherein the RNA is modified so as to confer upon the iRNA increased resistance to nuclease degradation, increased cellular uptake, and/or increased binding affinity for the target nucleic acid. An additional region of the iRNA may serve as a substrate for enzymes capable of cleaving RNA:DNA or RNA:RNA hybrids. By way of example, RNase H is a cellular endonuclease which cleaves the RNA strand of an RNA:DNA duplex.

Activation of RNase H, therefore, results in cleavage of the RNA target, thereby greatly enhancing the efficiency of iRNA inhibition of gene expression. Consequently, comparable results can often be obtained with shorter iRNAs when chimeric dsRNAs are used, compared to phosphorothioate deoxy dsRNAs hybridizing to the same target region. Cleavage of the RNA target can be routinely detected by gel electrophoresis and, if necessary, associated nucleic acid hybridization techniques known in the art.

In certain instances, the RNA of an iRNA can be modified by a non-ligand group. A number of non-ligand molecules have been conjugated to iRNAs in order to enhance the activity, cellular distribution or cellular uptake of the iRNA, and procedures for performing such conjugations are available in the scientific literature. Such non-ligand moieties have included lipid moieties, such as cholesterol (Kubo, T. et al., Biochem. Biophys. Res. Comm., 2007, 365(1):54-61; Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-S-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such RNA conjugates have been listed above. Typical conjugation protocols involve the synthesis of an RNAs bearing an aminolinker at one or more positions of the sequence. The amino group is then reacted with the molecule being conjugated using appropriate coupling or activating reagents. The conjugation reaction may be performed either with the RNA still bound to the solid support or following cleavage of the RNA, in solution phase. Purification of the RNA conjugate by HPLC typically affords the pure conjugate.

The delivery of an iRNA to a subject in need thereof can be achieved in a number of different ways. In vivo delivery can be performed directly by administering a composition comprising an iRNA, e.g. a dsRNA, to a subject. Alternatively, delivery can be performed indirectly by administering one or more vectors that encode and direct the expression of the iRNA. Absorption or uptake of an iRNA can occur through unaided diffusive or active cellular processes, or by auxiliary agents or devices. For example, for in vivo delivery, iRNA can be injected into a tissue site or administered systemically. In vivo delivery can also be by a beta-glucan delivery system, such as those described in U.S. Pat. Nos. 5,032,401 and 5,607,677, and U.S. Publication No. 2005/0281781, which are hereby incorporated by reference in their entirety. In vitro introduction into a cell includes methods known in the art such as electroporation and lipofection. Further approaches are described herein below or are known in the art.

In general, any method of delivering a nucleic acid molecule can be adapted for use with an iRNA (see e.g., Akhtar S. and Julian R L. (1992) Trends Cell. Biol. 2(5):139-144 and WO94/02595, which are incorporated herein by reference in their entireties). However, there are three factors that are important to consider in order to successfully deliver an iRNA molecule in vivo: (a) biological stability of the delivered molecule, (2) preventing non-specific effects, and (3) accumulation of the delivered molecule in the target tissue. The non-specific effects of an iRNA can be minimized by local administration, for example by direct injection or implantation into a tissue (as a non-limiting example, in adipose tissue) Local administration to a treatment site maximizes local concentration of the agent, limits the exposure of the agent to systemic tissues that may otherwise be harmed by the agent or that may degrade the agent, and permits a lower total dose of the iRNA molecule to be administered. Several studies have shown successful knockdown of gene products when an iRNA is administered locally. For example, intraocular delivery of a VEGF dsRNA by intravitreal injection in cynomolgus monkeys (Tolentino, M J., et al (2004) Retina 24:132-138) and subretinal injections in mice (Reich, S J., et al (2003) Mol. Vis. 9:210-216) were both shown to prevent neovascularization in an experimental model of age-related macular degeneration. In addition, direct intratumoral injection of a dsRNA in mice reduces tumor volume (Pille, J., et al (2005) Mol. Ther. 11:267-274) and can prolong survival of tumor-bearing mice (Kim, W J., et al (2006) Mol. Ther. 14:343-350; Li, S., et al (2007) Mol. Ther. 15:515-523). RNA interference has also shown success with local delivery to the CNS by direct injection (Dorn, G., et al. (2004) Nucleic Acids 32:e49; Tan, P H., et al (2005) Gene Ther. 12:59-66; Makimura, H., et al (2002) BMC Neurosci. 3:18; Shishkina, G T., et al (2004) Neuroscience 129:521-528; Thakker, E R., et al (2004) Proc. Natl. Acad. Sci. U.S.A. 101:17270-17275; Akaneya, Y., et al (2005) J. Neurophysiol. 93:594-602) and to the lungs by intranasal administration (Howard, K A., et al (2006) Mol. Ther. 14:476-484; Zhang, X., et al (2004) J. Biol. Chem. 279:10677-10684; Bitko, V., et al (2005) Nat. Med. 11:50-55). For administering an iRNA systemically for the treatment of a disease or condition (e.g. obesity), the RNA can be modified or alternatively delivered using a drug delivery system; both methods act to prevent the rapid degradation of the dsRNA by endo- and exo-nucleases in vivo. Modification of the RNA or the pharmaceutical carrier can also permit targeting of the iRNA composition to the target tissue and avoid undesirable off-target effects. iRNA molecules can be modified by chemical conjugation to lipophilic groups such as cholesterol to enhance cellular uptake and prevent degradation. For example, an iRNA directed against ApoB conjugated to a lipophilic cholesterol moiety was injected systemically into mice and resulted in knockdown of apoB mRNA in both the liver and jejunum (Soutschek, J., et al (2004) Nature 432:173-178). Conjugation of an iRNA to an aptamer has been shown to inhibit tumor growth and mediate tumor regression in a mouse model of prostate cancer (McNamara, J O., et al (2006) Nat. Biotechnol. 24:1005-1015). In an alternative embodiment, the iRNA can be delivered using drug delivery systems such as a nanoparticle, a dendrimer, a polymer, liposomes, or a cationic delivery system. Positively charged cationic delivery systems facilitate binding of an iRNA molecule (negatively charged) and also enhance interactions at the negatively charged cell membrane to permit efficient uptake of an iRNA by the cell. Cationic lipids, dendrimers, or polymers can either be bound to an iRNA, or induced to form a vesicle or micelle (see e.g., Kim S H., et al (2008) Journal of Controlled Release 129(2):107-116) that encases an iRNA. The formation of vesicles or micelles further prevents degradation of the iRNA when administered systemically. Methods for making and administering cationic-iRNA complexes are well within the abilities of one skilled in the art (see e.g., Sorensen, D R., et al (2003) J. Mol. Biol 327:761-766; Verma, U N., et al (2003) Clin. Cancer Res. 9:1291-1300; Arnold, A S et al (2007) J. Hypertens. 25:197-205, which are incorporated herein by reference in their entirety). Some non-limiting examples of drug delivery systems useful for systemic delivery of iRNAs include DOTAP (Sorensen, D R., et al (2003), supra; Verma, U N., et al (2003), supra), Oligofectamine, “solid nucleic acid lipid particles” (Zimmermann, T S., et al (2006) Nature 441:111-114), cardiolipin (Chien, P Y., et al (2005) Cancer Gene Ther. 12:321-328; Pal, A., et al (2005) Int J. Oncol. 26:1087-1091), polyethyleneimine (Bonnet M E., et al (2008) Pharm. Res. August 16 Epub ahead of print; Aigner, A. (2006) J. Biomed. Biotechnol. 71659), Arg-Gly-Asp (RGD) peptides (Liu, S. (2006) Mol. Pharm. 3:472487), and polyamidoamines (Tomalia, D A., et al (2007) Biochem. Soc. Trans. 35:61-67; Yoo, H., et al (1999) Pharm. Res. 16:1799-1804). In some embodiments, an iRNA forms a complex with cyclodextrin for systemic administration. Methods for administration and pharmaceutical compositions of iRNAs and cyclodextrins can be found in U.S. Pat. No. 7,427,605, which is herein incorporated by reference in its entirety.

In another aspect, iRNA can be expressed from transcription units inserted into DNA or RNA vectors (see, e.g., Couture, A, et al., TIG. (1996), 12:5-10; Skillern, A., et al., International PCT Publication No. WO 00/22113, Conrad, International PCT Publication No. WO 00/22114, and Conrad, U.S. Pat. No. 6,054,299). Expression can be transient (on the order of hours to weeks) or sustained (weeks to months or longer), depending upon the specific construct used and the target tissue or cell type. These transgenes can be introduced as a linear construct, a circular plasmid, or a viral vector, which can be an integrating or non-integrating vector. The transgene can also be constructed to permit it to be inherited as an extrachromosomal plasmid (Gassmann, et al., Proc. Natl. Acad. Sci. USA (1995) 92:1292).

The individual strand or strands of an iRNA can be transcribed from a promoter on an expression vector. Where two separate strands are to be expressed to generate, for example, a dsRNA, two separate expression vectors can be co-introduced (e.g., by transfection or infection) into a target cell. Alternatively each individual strand of a dsRNA can be transcribed by promoters both of which are located on the same expression plasmid. In one embodiment, a dsRNA is expressed as inverted repeat polynucleotides joined by a linker polynucleotide sequence such that the dsRNA has a stem and loop structure.

iRNA expression vectors are generally DNA plasmids or viral vectors. Expression vectors compatible with eukaryotic cells, preferably those compatible with vertebrate cells, can be used to produce recombinant constructs for the expression of an iRNA as described herein. Eukaryotic cell expression vectors are well known in the art and are available from a number of commercial sources. Typically, such vectors are provided containing convenient restriction sites for insertion of the desired nucleic acid segment. Delivery of iRNA expressing vectors can be systemic, such as by intravenous or intramuscular administration, by administration to target cells ex-planted from the patient followed by reintroduction into the patient, or by any other means that allows for introduction into a desired target cell.

Vectors useful for the delivery of an iRNA will include regulatory elements (promoter, enhancer, etc.) sufficient for expression of the iRNA in the desired target fungal cell. The regulatory elements can be chosen to provide either constitutive or regulated/inducible expression.

Expression of the iRNA can be precisely regulated, for example, by using an inducible regulatory sequence that is sensitive to certain physiological regulators, e.g., circulating glucose levels, or hormones (Docherty et al., 1994, FASEB J. 8:20-24). Such inducible expression systems, suitable for the control of dsRNA expression in cells include, for example, regulation by ecdysone, by estrogen, progesterone, tetracycline, chemical inducers of dimerization, and isopropyl-beta-D1-thiogalactopyranoside (IPTG). A person skilled in the art would be able to choose the appropriate regulatory/promoter sequence based on the intended use of the iRNA transgene.

The pharmaceutical preparation of a vector can include the vector in an acceptable diluent, or can include a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

In some embodiments, the iRNA can be delivered via a liposome. As used in the present invention, the term “liposome” means a vesicle composed of amphiphilic lipids arranged in a spherical bilayer or bilayers. A number of liposomes comprising nucleic acids are known in the art. WO 96/40062 to Thierry et al. discloses methods for encapsulating high molecular weight nucleic acids in liposomes. U.S. Pat. No. 5,264,221 to Tagawa et al. discloses protein-bonded liposomes and asserts that the contents of such liposomes may include a dsRNA. U.S. Pat. No. 5,665,710 to Rahman et al. describes certain methods of encapsulating oligodeoxynucleotides in liposomes. WO 97/04787 to Love et al. discloses liposomes comprising dsRNAs targeted to the raf gene.

In one embodiment, an iRNA as described herein is fully encapsulated in the lipid formulation, e.g., to form a SPLP, pSPLP, SNALP, or other nucleic acid-lipid particle. As used herein, the term “SNALP” refers to a stable nucleic acid-lipid particle, including SPLP. As used herein, the term “SPLP” refers to a nucleic acid-lipid particle comprising plasmid DNA encapsulated within a lipid vesicle. SNALPs and SPLPs typically contain a cationic lipid, a non-cationic lipid, and a lipid that prevents aggregation of the particle (e.g., a PEG-lipid conjugate). SNALPs are described, e.g., in U.S. Patent Application Publication Nos. 20060240093, 20070135372, and in International Application No. WO 2009082817. These applications are incorporated herein by reference in their entirety.

In some embodiments, the iRNA can be targeted, e.g. targeted to fungal cells. Targeted delivery of iRNAs is described, for example in Ikeda and Taira Pharmaceutical Res 2006 23:1631-1640; which is incorporated by reference herein in its entirety. By way of example, the inhibitor can be targeted to fungal cells by encapsulating the inhibitor in a liposome comprising receptors of ligands expressed on fungal cells, e.g. the vertebrate receptors dectin-1; CR3, CD5, CD36, SCARF1, CD206, DC-SIGN, dectin-2, TLR2, and TLR4.

As demonstrated herein, modulation of target as described herein potentiates the activity and efficacy of antifungal agents. Accordingly, provided herein in some aspects, are compositions, such as therapeutic compositions, comprising an effective amount of one or more potentiator compounds (e.g. one potentiator compound, two potentiator compounds, three potentiator compounds, or more potentiator compounds), as described herein, and an effective amount of an antifungal agent. Where a plurality of potentiator compounds are used in accordance with the methods and compositions described herein, the potentiator compounds can be from the same class of potentiator compounds (e.g., two or more phosphodiesterase inhibitors) or multiple classes of potentiator compounds (e.g. a phosphodiesterase compound and cAMP).

As used herein, the term “antifungal” refers to any compound known to one of ordinary skill in the art that will inhibit or reduce the growth of, or kill, one or more fungal species. Thus, the ability to inhibit or reduce the growth of, or kill, one or more fungal organisms is referred to herein as “antifungal activity.” In some embodiments, an antifungal agent for use in the compositions and methods described herein is “fungistatic,” meaning that they stop fungi from reproducing, while not necessarily harming them otherwise. Fungistatic agents limit the growth of fungi by interfering with fungi protein production, DNA replication, or other aspects of fungal cellular metabolism, and typically work together with the immune system to remove fungi from the body. High concentrations of some fungistatic agents are also fungicidal, in some cases, whereas low concentrations of some fungicidal agents are fungistatic. In some embodiments, an antifungal agent (or the effective amount thereof) for use in the compositions and methods described herein is “fungicidal” for the target fungus. That is, the agent kills the target fungal cells and, ideally, is not substantially toxic to mammalian cells. Fungicidal agents include disinfectants, and antiseptics. Many antifungal compounds are relatively small molecules with a molecular weight of less than 2000 atomic mass units. The term “antifungal” includes includes, but is not limited to the antifungals described herein or any salts or variants thereof. The antifugnal used in addition to the potentiator compound in the various embodiments of the compositions and methods described herein will depend on the type of fungal infection.

Any of the major classes of antifungal agents in which fungicidal activity is potentiated or enhanced by, e.g. increasing ROS production, can be used with the potentiator compounds described herein.

Such classes of antifungal agents include, for example, polyenes, imidazoles, triazoles, thiazoles, allylamine, and echinocandins. Accordingly, non-limiting examples of antifungal agents that are suitable for use with the compositions and methods described herein, provided they can be potentiated by modulation of a target, include, without limitation, amphotericin B, candicidin, filipin, hamycin, natamycin, nystatin, rimocidin, bifonazole, butoconazole, clotrimazole, econazole, fenticonzole, isoconazole, ketoconazole, miconazole, omoconazole, oxiconazole, sertaconazole, sulconazole, tioconazole, albaconazole; fluconazole; isavuconazole; itraconazole; posaconazole; ravuconazole; terconazole; voriconazole, abafungin, amorolfin; butenafine; naftifine; terbinafine, anidulafungin; caspofungin; and micafungin.

Antifungal polyenes are macrocyclic polyenes with a heavily hydroxylated region on the ring opposite the conjugated system, rendering them amphiphilic. Polyenes act by binding to sterols, e.g. ergosterol, in the fungal membrane, making the membrane more crystalline. The polyene, amphotericin B (AMB), introduced in the late 1950s, was the first widely utilized antifungal (AF) drug. Due to its strong hydrophobicity, AMB penetrates the fungal membrane and binds to ergosterol leading to membrane damage. Non-limiting examples of polyenes can include amphotericin B; candicidin; filipin; hamycin; natamycin; nystatin; and rimocidin.

Azoles inhibit ergosterol biosynthesis and lead to the accumulation of a toxic methylated sterol that stops cell growth. While azoles tend to be fungistatic due to their poor solubility, under certain conditions and formulations, azoles such as miconazole (MCZ) can be fungicidal. Non-limiting examples of imidazoles can include bifonazole; butoconazole; clotrimazole; econazole; fenticonzole; isoconazole; ketoconazole; miconazole; omoconazole; oxiconazole; sertaconazole; sulconazole; and tioconazole. Non-limiting examples of triazoles can include albaconazole; fluconazole; isavuconazole; itraconazole; posaconazole; ravuconazole; terconazole; and voriconazole. In some embodiments, the antifungal agent can be a thiazole, e.g. abafungin.

Echinocandins inhibit the synthesis of cell wall glucan. Non-limiting examples of echinocandins can include anidulafungin; caspofungin; and micafungin.

Allylamines inhibit squalene epoxidase, which is required for ergosterol biosynthesis. Non-limiting examples of allylamines can include amorolfin; butenafine; naftifine; and terbinafine

Further non-limiting examples of antifungal agents can include benzoic acid; ciclopirox; flucytosine; griseofulvin; haloprogin; polygodial; tolnaftate; undecylenic acid; and crystal violet.

### Potentiator Compounds and Methods of Treatment or Inhibition of Fungal Infections Thereof

As demonstrated herein, contacting with or administering an effective amount of one or more potentiator compounds with an effective amount of an antifungal agent that, e.g. increases ROS production and/or inhibits DSB repair as part of its antifungal activity can be used in methods of treatment or inhibition of fungal infections and/or fungal growth.

Accordingly, in some aspects, provided herein are methods for treating or inhibiting a fungal infection (i.e. mycosis), the methods comprising administering to a subject having or at risk for a fungal infection an effective amount of at least one potentiator compound and an effective amount of an antifungal agent. The methods described herein can, in some aspects and embodiments, be used to inhibit, delay formation of, treat, and/or prevent or provide prophylactic treatment of fungal infections in animals, including humans.

As used herein, the terms “inhibit”, “decrease,” “reduce,” “inhibiting” and “inhibition” have their ordinary and customary meanings to generally mean a decrease by a statistically significant amount, and include inhibiting the growth or cell division of a fungal cell or fungal cell population, as well as killing such fungi. Such inhibition is an inhibition of about 20% to about 100% of the growth of the fungus versus the growth of fungi in the presence of the antifungal agent, but in the absence of the effective amount of the one or more potentiator compounds. Preferably, the inhibition is an inhibition of about at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or more, of the growth or survival of the fungi in comparison to a reference or control level in the absence of the effective amount of the one or more potentiator compounds.

The methods described herein are applicable to the treatment of human and non-human subjects or individuals. The terms “subject” and “individual” are used interchangeably herein, and refer to an animal, for example a human, recipient of the one or more potentiator compounds and antifungal agent, such as, for example, cAMP and amphotericin. For treatment of those disease states which are specific for a specific animal, such as a human subject, the term “subject” refers to that specific animal. The terms ‘non-human animals’ and ‘non-human mammals’ are used interchangeably herein, and include mammals such as rats, mice, rabbits, sheep, cats, dogs, cows, horses, pigs, and non-human primates. In some embodiments, the subject is a veterinary patient such as a dog or cat. The term “subject” can also encompass any vertebrate including but not limited to mammals, reptiles, amphibians and fish.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder, such as a fungal infection, and include one or more of: ameliorating a symptom of a fungal infection in a subject; blocking or ameliorating a recurrence of a symptom of a fungal infection; decreasing in severity and/or frequency a symptom of a fungal infection in a subject; and stasis, decreasing, or inhibiting growth of a fungal infection in a subject. Treatment means ameliorating, blocking, reducing, decreasing or inhibiting by about 1% to about 100% versus a subject to whom the effective amount of the one or more potentiator compounds and antifungal agent has not been administered. Preferably, the ameliorating, blocking, reducing, decreasing or inhibiting is about at least 5%, at least 10%, at least 15%, at least 20%, at least 25%, at least 300, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or more, versus a subject to whom the effective amount of the one or more potentiator compounds and antifungal agent has not been administered. Treatment is generally considered “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment).

As used herein, the phrase “alleviating a symptom of a fungal infection” is ameliorating any condition or symptom associated with the infection. Alternatively, alleviating a symptom of a fungal infection can involve reducing the infectious fungal load in the subject relative to such load in an untreated control. As compared with an equivalent untreated control, such reduction or degree of prevention is at is about at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 900%, at least 95%, at least 98%, at least 990%, or more, as measured by any standard technique. Desirably, the fungal infection is completely cleared as detected by any standard method known in the art, in which case the persistent infection is considered to have been treated. A patient who is being treated, for example, for a persistent infection is one who a medical practitioner has diagnosed as having such a condition. Diagnosis can be by any suitable means. Diagnosis and monitoring can involve, for example, detecting the level of fungal load in a biological sample (for example, a tissue biopsy, blood test, or urine test), detecting the level of a surrogate marker of the fungal infection in a biological sample, detecting symptoms associated with the infection, or detecting immune cells involved in the immune response typical of fungal infections (for example, detection of antigen specific T cells or antibody production).

As used herein, the terms “preventing” and “prevention” have their ordinary and customary meanings, and include one or more of: preventing an increase in the growth of a population of fungi in a subject, or on a surface or on a porous material; preventing development of a disease caused by a fungus in a subject; and preventing symptoms of an infection or disease caused by a fungal infection in a subject. As used herein, the prevention lasts at least about 0.5 days, 1 day, 2 days, 3 days, 4 days, 5 days, 6 days, 7 days, 10 days, 12 days, 15 days, 20 days, 25 days, 30 days, 35 days, 40 days or more days after administration or application of the effective amount of the one or more potentiator compounds and antifungal agent, as described herein.

Accordingly, in some aspects, provided herein are methods for inhibiting a fungal infection, the methods comprising administering to a patient having or at risk for a fungal infection an effective amount of one or more potentiator compounds and an effective amount of an antifungal agent.

In some aspects, provided herein are methods for preventing a fungal infection, the methods comprising administering to a patient having or at risk for a fungal infection an effective amount of one or more potentiator compounds and an effective amount of an antifungal agent.

In some aspects, provided herein are methods for inhibiting a fungal infection, the methods comprising administering to a patient having or at risk for a fungal infection an effective amount of a pharmaceutical composition comprising one or more potentiator compounds and an antifungal agent.

In some aspects, provided herein are methods for preventing a fungal infection, the methods comprising administering to a patient having or at risk for a fungal infection an effective amount of a pharmaceutical composition comprising one or more potentiator compounds and an antifungal agent.

Also provided herein, in some aspects, are methods for treating a fungal infection, comprising: administering to a patient having a fungal infection and undergoing treatment with an antifungal agent an effective amount of one or more potentiator compounds. A subject underoing treatment with an antifungal agent can be a subject who has or who is at risk of having a fungal infection and who has been administered an antifungal agent as described herein, without limitation as to the dose or route of administration. In some embodiments, a subject is undergoing treatment if they are scheduled to be administered, or having been prescribed to be administered a future dose of an antifungal agent. In some embodiments, a subject is undergoing treatment if they have been administered a dose of an antifungal agent within the prior week, e.g. the prior 5 days, the prior 3 days, the prior 2 days, or the previous day.

The potentiator compounds described herein that potentiate and improve antifungal efficacy, as exemplified in C. albicans, can be effective for, increasing antifungal sensitivity by, e.g. increasing ROS production in a variety of fungal species, including, but not limited to, Candida spp.; Cryptococcus spp.; Aspergillus spp.; Microsporum spp.; Trichophyton spp.; Epidermophyton spp.; Trichosporon spp.; Fusarium spp.; Tinea versicolor; Tinea barbae; Tinea corporis; Tinea cruris; Tinea manuum; Tinea pedis; Tinea unguium; Tineafaciei; Tinea imbricate; Tinea incognito; Epidermophyton floccosum; Microsporum canis; Microsporum audouinii; Trichophyton interdigitale; Trichophyton mentagrophytes; Trichophyton tonsurans; Trichophyton schoenleini; Trichophyton rubrum; Hortaea werneckii; Piedraia hortae; Malasserzia furfur; Coccidioides immitis; Coccidioides posadasii; Histoplasma capsulatum; Histoplasma duboisii; Lacazia loboi; Paracoccidioides brasiliensis; Blastomyces dermatitidis; Sporothrix schenckii; Penicillium marneffei; Candida albicans; Candida glabrata; Candida tropicalis; Candida lusitaniae; Candidajirovecii; Candida krusei; Candida parapsilosi; Exophialajeanselmei; Fonsecaea pedrosoi; Fonsecasea compacta; Phialophora verrucosa; Geotrichum candidum; Pseudallescheria boydii; Rhizopus oryzae; Muco indicus; Absidia corymbifera; Synceplasastrum racemosum; Basidiobolus ranarum; Conidiobolus coronatus; Conidiobolus incongruous; Cryptococcus neoformans; Enterocytozoan bieneusi; Encephalitozoon intestinalis; and Rhinosporidium seeberi, according to the compositions and methods described herein. Accordingly, the potentiator compounds are effective at improving and enhancing the treatment of various disorders and diseases caused by fungal infections or toxins produced during such infections. Such fungal infections include those caused by fungi having a similar metabolic system to Candida albicans. In some embodiments, the fungus inhibited by the methods and compositions described herein is not C. neoformans. As used herein, a “fungal infection” refers to an abnormal and/or undesired presence of a fungus in or on a subject. The presence can be abnormal in that the fungus is a noncommensal species, e.g. one not typically found in or on a healthy subject, or it can be abnormal in that the fungus is present at at abnormally high levels, e.g. at least twice the level found in or on a healthy subject (e.g. twice the level, three times the level, four times the level, five times the level, or greater), or it can be abnormal in that the presence of the fungus is causing or contributing to disease or symptoms thereof, e.g. necrosis, disfigurement, delayed wound healing, etc.

Non-limiting examples of disorders/diseases caused by fungal infections or toxins produced during fungal infections, and for which the compositions and methods described herein are applicable in various aspects and embodiments, include, but are not limited to, infection of a surface wound or burn; infection of a mucosal surface; respiratory infection; infections of the eyes, ears, nose, or throat; or infection of an intestinal pathogen. In other embodiments, the disorder or disease is an infection of soft tissue or skin, such as a superficial mycosis; a cutaneous mycosis; a subcutaneous mycosis; a vaginal mycosis; a systemic mycosis; or is an infected wound or burn.

Accordingly, in various embodiments of methods and compositions and methods described herein, the combination of antifungal agent and one or more potentiator compounds administered or used is determined based on the nature of the fungal infection, for example, whether an acute or chronic infection, in the subject.

Non-limiting examples of infectious fungi causing fungal infections that are contemplated for use with the combinatorial therapeutic compositions and methods described herein include, but are not limited to: Candida spp.; Cryptococcus spp.; Aspergillus spp.; Microsporum spp.; Trichophyton spp.; Epidermophyton spp.; Trichosporon spp.; Tinea versicolor; Tinea barbae; Tinea corporis; Tinea cruris; Tinea manuum; Tinea pedis; Tinea unguium; Tineafaciei; Tinea imbricate; Tinea incognito; Epidermophyton floccosum; Microsporum canis; Microsporum audouinii; Trichophyton interdigitale; Trichophyton mentagrophytes; Trichophyton tonsurans; Trichophyton schoenleini; Trichophyton rubrum; Hortaea werneckii; Piedraia hortae; Malasserzia furfur; Coccidioides immitis; Coccidioides posadasii; Histoplasma capsulatum; Histoplasma duboisii; Lacazia loboi; Paracoccidioides brasiliensis; Blastomyces dermatitidis; Sporothrix schenckii; Penicillium marneffei; Candida albicans; Candida glabrata; Candida tropicalis; Candida lusitaniae; Candidajirovecii; Exophialajeanselmei; Fonsecaea pedrosoi; Fonsecasea compacta; Phialophora verrucosa; Geotrichum candidum; Pseudallescheria boydii; Rhizopus oryzae; Muco indicus; Absidia corymbifera; Synceplasastrum racemosum; Basidiobolus ranarum; Conidiobolus coronatus; Conidiobolus incongruous; Cryptococcus neoformans; Enterocytozoan bieneusi; Encephalitozoon intestinalis; and Rhinosporidium seeberi.

Also provided herein in some embodiments and aspects of the compositions and methods described herein, are synergistic combinations of potentiator compounds and antifungal agents for the treatment of fungal infections exhibiting antifungal or drug resistance. In some embodiments of the aspects described herein, the infection is caused by a fungal species that exhibits antifungal resistance.

In some embodiments of the aspects described herein, the methods of treating a subject having or at increased risk for a fungal infection, further comprise the step of selecting, diagnosing, or identifying a subject having or at increased risk for a fungal infection. In such embodiments, a subject is identified as having a fungal infection by objective determination of the presence of fungal cells in the subject's body by one of skill in the art. Such objective determinations can be performed through the sole or combined use of tissue analyses, blood analyses, urine analyses, and fungal cell cultures, in addition to the monitoring of specific symptoms associated with the fungal infection.

In some embodiments of the methods described herein, the infection is an “acute” or “non-latent infection,” that is, an infection where the fungi is actively or aggressively proliferating, and typically having a relatively short time course of infection. Such infections can require aggressive antifungal intervention. Such infections are often termed “acute,” and lead to quickly advancing disease. Acute infections typically begin with an incubation period, during which the fungi replicate and host innate immune responses are initiated. The cytokines produced early in infection lead to classical symptoms of an acute infection: aches, pains, fever, malaise, and nausea. Once an acute infection is cleared, the infectious agent cannot be detected in the subject. Acute infections, as used herein, do not enter a latent phase where the fungal agent is present but the subject is non-symptomatic. In some embodiments, an acute infection is one in which the subject has one or more active symptoms of infection, e.g., aches, pains, fever, malaise, nausea, active/proliferating fungal cells, active/proliferating immune cells, detectable levels of one or more cytokines in the circulation, etc.

Accordingly, in some embodiments of these methods and all such methods described herein, provided herein are methods of inhibiting or preventing an acute infection in a subject before, during, or after an invasive medical treatment, comprising administering to a subject before, during, and/or after an invasive medical treatment an effective amount of one or more potentiator compounds and an effective amount of an antifungal agent.

Such methods can be used for achieving a systemic and/or local effect against relevant fungi shortly before or after an invasive medical treatment, such as surgery or insertion of an in-dwelling medical device (e.g. joint replacement (hip, knee, shoulder, etc.)). Treatment can be continued after invasive medical treatment, such as post-operatively or during the in-body time of the device.

In some such embodiments, the one or more potentiator compounds and the antifungal agent can be administered once, twice, thrice or more, from 1 day, 2 days, 3 days, 4 days, 5 days, 6 days, 7 days or more, to 10 hours, 9 hours, 8 hours, 7 hours, 6 hours, 5 hours, 4 hours, 3 hours, 2 hours, or 1 hour or immediately before surgery for permitting a systemic or local presence of the antifungal agent in combination with the one or more potentiator compounds. The pharmaceutical composition(s) comprising the antifungal agent and the one or more potentiator compounds can, in some embodiments, be administered after the invasive medical treatment for a period of time, such as 1 day, 2 days, 3 days, 4 days, 5 days or 6 days, 1 week, 2 weeks, 3 weeks or more, or for the entire time in which the device is present in the body of the subject. As used herein, the term “bi-weekly” refers to a frequency of every 13-15 days, the term “monthly” refers a frequency of every 28-31 days and “bi-monthly” refers a frequency of every 58-62 days.

In some embodiments of these methods, the surface of the in-dwelling device is coated by a solution, such as through bathing or spraying, containing a concentration of about 1 μg/ml to about 500 mg/ml of the antifungal agent and one or more potentiator compounds described herein. When being applied to an in-dwelling medical device, the surface can be coated by a solution comprising the antifungal agent and one or more potentiator compounds before its insertion in the body.

In other embodiments of the methods described herein, the fungal infection is a persistent or a chronic fungal infection.

As used herein, “persistent infections” refer to those infections that, in contrast to acute infections, are not effectively or completely cleared by a host immune response or by antifungal administration. Persistent infections include for example, latent, chronic and slow infections. In a “chronic infection,” the infectious agent can be detected in the subject at all times. However, the signs and symptoms of the disease can be present or absent for an extended period of time. Non-limiting examples of chronic infections include a variety of fungal infections, as described herein below, as well as secondary fungal infections resulting from or caused by infection with another agent that suppresses or weakens the immune system, such as chronic viral infections, such as, for example, hepatitis B (caused by hepatitis B virus (HBV)) and hepatitis C (caused by hepatitis C virus (HCV)) adenovirus, cytomegalovirus, Epstein-Barr virus, herpes simplex virus 1, herpes simplex virus 2, human herpesvirus 6, varicella-zoster virus, hepatitis B virus, hepatitis D virus, papilloma virus, parvovirus B19, polyomavirus BK, polyomavirus JC, measles virus, rubella virus, human immunodeficiency virus (HIV), human T cell leukemia virus I, and human T cell leukemia virus II, as well as secondary fungal infections resulting from or caused by infection with a persistent parasitic persistent infection, such as, for example, Leishmania, Toxoplasma, Trypanosoma, Plasmodium, Schistosoma, and Encephalitozoon.

Because most antifungal agents exert maximal activity against rapidly dividing cells, antifungal therapies for these infections are not optimal, requiring protracted treatment times, high and sometimes toxic antifungal doses, and demonstrating higher failure rates. In contrast, the novel methods and compositions described herein, which combine an effective amount of one or more potentiator compounds to potentiate the efficacy and fungicidal activity of an antifungal agent, permits increased efficacy of the antifungal agent and enhanced susceptibility of the fungi to the agent.

The terms “persistent cell” or “persister fungal cells” are used interchangeably herein and refer to a metabolically dormant subpopulation of fungal cells, which are not sensitive to antimicrobial agents such as antifungals. Persisters typically are not responsive, i.e. are not killed or inhibited by antifungals, as they have, for example, non-lethally downregulated the pathways on which the antifungals act. Persisters can develop at non-lethal (or sub-lethal) concentrations of the antifungal agent.

Accordingly, in some aspects, provided herein are methods of inhibiting or preventing formation or colonization of a persistent, slow growing, and/or stationary-phase fungus in a subject before, during, or after an invasive medical treatment, comprising administering to a subject before, during, and/or after an invasive medical treatment an effective amount of one or more potentiator compounds and an effective amount of an antifungal agent.

Such methods can be used for achieving a systemic and/or local effect against relevant fungi shortly before or after an invasive medical treatment, such as surgery or insertion of an in-dwelling medical device (e.g. joint replacement (hip, knee, shoulder, etc.)). Treatment can be continued after invasive medical treatment, such as post-operatively or during the in-body time of the device.

In some such embodiments, the one or more potentiator compounds and the antifungal agent can be administered once, twice, thrice or more, from 1 day, 2 days, 3 days, 4 days, 5 days, 6 days, 7 days or more, to 10 hours, 9 hours, 8 hours, 7 hours, 6 hours, 5 hours, 4 hours, 3 hours, 2 hours, or 1 hour or immediately before surgery for permitting a systemic or local presence of the antifungal agent in combination with the one or more potentiator compounds. The pharmaceutical composition(s) comprising the antifungal agent and the one or more potentiator compounds can, in some embodiments, be administered after the invasive medical treatment for a period of time, such as 1 day, 2 days, 3 days, 4 days, 5 days or 6 days, 1 week, 2 weeks, 3 weeks or more, or for the entire time in which the device is present in the body of the subject. As used herein, the term “bi-weekly” refers to a frequency of every 13-15 days, the term “monthly” refers a frequency of every 28-31 days and “bi-monthly” refers a frequency of every 58-62 days.

In some embodiments of these methods, the surface of the in-dwelling device is coated by a solution, such as through bathing or spraying, containing a concentration of about 1 gig/ml to about 500 mg/ml of the antifungal agent and one or more potentiator compounds described herein. When being applied to an in-dwelling medical device, the surface can be coated by a solution comprising the antifungal agent and one or more potentiator compounds before its insertion in the body.

In some embodiments of the methods described herein, a subject refers to a human subject having a chronic infection or at increased risk for a chronic infection. A subject that has a chronic infection is a subject having objectively measurable fungal cells present in the subject's body. A subject that has increased risk for a chronic infection includes subjects with an in-dwelling medical device, for example, or a subject having or having had a surgical intervention.

In some embodiments of the methods described herein, the subject having or at risk for a chronic infection is an immunocompromised subject, such as, for example, HIV-positive patients, who have developed or are at risk for developing a fungal infection from either an opportunistic infection or from the reactivation of a suppressed or latent infection; subjects with cystic fibrosis, chronic obstructive pulmonary disease, and other such immunocompromised and/or institutionalized patients.

Also provided herein, in some aspects, are methods of inhibiting or delaying the formation of biofilms, comprising administering to a subject in need thereof or contacting a surface with an effective amount of one or more potentiator compounds and an antifungal agent in combination.

As used herein, a “biofilm” refers to mass of microorganisms attached to a surface, such as a surface of a medical device, and the associated extracellular substances produced by one or more of the attached microorganisms. The extracellular substances are typically polymeric substances that commonly include a matrix of complex polysaccharides, proteinaceous substances and glycopeptides. The microorganisms can include, but are not limited to, bacteria, fungi and protozoa. In a “fungal biofilm,” the microorganisms include one or more species of fungi. The nature of a biofilm, such as its structure and composition, can depend on the particular species of fungus present in the biofilm. Fungi present in a biofilm are commonly genetically or phenotypically different than corresponding fungi not in a biofilm, such as isolated fungi or fungi in a colony. “Polymicrobic biofilms” are biofilms that include a plurality of fungal species.

As used herein, the terms and phrases “delaying”, “delay of formation”, and “delaying formation of” have their ordinary and customary meanings, and are generally directed to increasing the period of time prior to the formation of biofilm, or a slow growing fungal infection in a subject or on a surface. The delay may be, for example, about 12 hours, about 18 hours, about 24 hours, about 36 hours, about 48 hours, about 60 hours, about 72 hours, about 84 hours, about 96 hours, about 5 days, 6 days, 7 days, 8 days, 9 days, 10 days, or more. Inhibiting formation of a biofilm, as used herein, refers to avoiding the partial or full development or progression of a biofilm, for example, on a surface, such as a surface of an indwelling medical device.

The skilled artisan will understand that the methods of inhibiting and delaying the formation of biofilms can be practiced wherever persistent, slow-growing, stationary-phase, or biofilm forming fungi, can be encountered. For example, the methods described herein can be practiced on the surface of or inside of an animal, such as a human; on an inert surface, such as a counter or bench top; on a surface of a piece of medical or laboratory equipment; on a surface of a medical or laboratory tool; or on a surface of an in-dwelling medical device.

Accordingly, in some embodiments, the methods described herein further encompass surfaces coated by one or more potentiator compounds and an antifungal agent, and/or impregnated with one or more potentiator compounds and an antifungal agent. Such surfaces include any that can come into contact with a perisistent, slow growing, stationary-phase, biofilm fungi. In some such embodiments, such surfaces include any surface made of an inert material (although surfaces of a living animal are encompassed within the scope of the methods described herein), including the surface of a counter or bench top, the surface of a piece of medical or laboratory equipment or a tool, the surface of a medical device such as a respirator, and the surface of an in-dwelling medical device. In some such embodiments, such surfaces include those of an in-dwelling medical device, such as surgical implants, orthopedic devices, prosthetic devices and catheters, i.e., devices that are introduced to the body of an individual and remain in position for an extended time. Such devices include, but are not limited to, artificial joints, artificial hearts and implants; valves, such as heart valves; pacemakers; vascular grafts; catheters, such as vascular, urinary and continuous ambulatory peritoneal dialysis (CAPD) catheters; shunts, such as cerebrospinal fluid shunts; hoses and tubing; plates; bolts; valves; patches; wound closures, including sutures and staples; dressings; and bone cement.

As used herein, the term “indwelling medical device,” refers to any device for use in the body of a subject, such as intravascular catheters (for example, intravenous and intra-arterial), right heart flow-directed catheters, Hickman catheters, arteriovenous fistulae, catheters used in hemodialysis and peritoneal dialysis (for example, silastic, central venous, Tenckhoff, and Teflon catheters), vascular access ports, indwelling urinary catheters, urinary catheters, silicone catheters, ventricular catheters, synthetic vascular prostheses (for example, aortofemoral and femoropopliteal), prosthetic heart valves, prosthetic joints, orthopedic implants, penile implants, shunts (for example, Scribner, Torkildsen, central nervous system, portasystemic, ventricular, ventriculoperitoneal), intrauterine devices, tampons, dental implants, stents (for example, ureteral stents), artificial voice prostheses, tympanostomy tubes, gastric feeding tubes, endotracheal tubes, pacemakers, implantable defibrillators, tubing, cannulas, probes, blood monitoring devices, needles, and the like. A subcategory of indwelling medical devices refer to implantable devices that are typically more deeply and/or permanently introduced into the body. Indwelling medical devices can be introduced by any suitable means, for example, by percutaneous, intravascular, intraurethral, intraorbital, intratracheal, intraesophageal, stromal, or other route, or by surgical implantation, for example intraarticular placement of a prosthetic joint.

In some aspects, provided herein are methods of inhibiting the formation of a biofilm on a surface or on a porous material, comprising applying to or contacting a surface or a porous material upon which a biofilm can form one or more potentiator compounds and an antifungal agent in amounts sufficient to inhibit the formation of a biofilm. In some embodiments of these methods and all such methods described herein, the surface is an inert surface, such as the surface of an in-dwelling medical device.

In some aspects, provided herein are methods of preventing the colonization of a surface by persistent fungi, comprising applying to or contacting a surface with one or more potentiator compounds and an antifungal agent in an amount(s) sufficient to prevent colonization of the surface by persistent fungi.

In some aspect, provided herein is a method for inhibiting fungal growth, the method comprising contacting a fungal cell with an effective amount of one or more potentiator compounds and an effective amount of an antifungal agent.

As used herein, the term “contacting” is meant to broadly refer to bringing a fungal cell and one or more potentiator compounds and an antifungal agent into sufficient proximity that the one or more potentiator compounds and the antifungal agent can exert their effects on any fungal cell present. The skilled artisan will understand that the term “contacting” includes physical interaction between the one or more Potentiator compounds and the antifungal agent and a fungal cell, as well as interactions that do not require physical interaction.

In the embodiments of the methods described herein directed to inhibiting or delaying the formation of a biofilm, or preventing the colonization of a surface by persistent fungi, the material comprising the surface or the porous material can be any material that can be used to form a surface or a porous material. In some such embodiments, the material is selected from: polyethylene, polytetrafluoroethylene, polypropylene, polystyrene, polyacrylamide, polyacrylonitrile, poly(methyl methacrylate), polyamide, polyester, polyurethane, polycarbornate, silicone, polyvinyl chloride, polyvinyl alcohol, polyethylene terephthalate, cobalt, a cobalt-base alloy, titanium, a titanium base alloy, steel, silver, gold, lead, aluminum, silica, alumina, yttria stabilized zirconia polycrystal, calcium phosphate, calcium carbonate, calcium fluoride, carbon, cotton, wool and paper.

In some embodiments of these methods and all such methods described herein, the persistent, slow growing, stationary-phase or biofilm fungi is any fungal species or population that comprises persistent cells, can exist in a slow growing or stationary-phase, and/or that can form a biofilm.

Dosing and Modes of Administration

One key advantage of the methods, uses and compositions comprising the one or more potentiator compounds and an antifungal agent described herein, is the ability of producing marked anti-fungal effects in a human subject having a fungal infection and thereby increasing fungal sensitivity and susceptibility to a variety of antifungal classes, as well as reducing toxicities and adverse effects. By adding potentiator compounds to a therapeutic regimen or method, the dosage of the antifungal being administered can, in some embodiments, be reduced relative to the normally administered dosage. The efficacy of the treatments and methods described herein can be measured by various parameters commonly used in evaluating treatment of infections, including but not limited to, reduction in rate of fungal growth, the presence or number of fungal cells in a sample obtained from a subject, overall response rate, duration of response, and quality of life.

Accordingly, a “therapeutically effective amount” or “effective amount” of a potentiator compound, formulated alone or in combination with an antifungal agent, to be administered to a subject is governed by various considerations, and, as used herein, refers to the minimum amount necessary to prevent, ameliorate, or treat, or stabilize, a disorder or condition (e.g. a fungal infection). An effective amount as used herein also includes an amount sufficient to delay the development of a symptom of a fungal infection, alter the course of a fungal infection (for example but not limited to, slow the progression of a symptom of the fungal infection, such as growth of the fungal population), or reverse a symptom of the fungal infection.

Effective amounts, toxicity, and therapeutic efficacy of the potentiator compound, formulated alone or in combination with an antifungal agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dosage can vary depending upon the dosage form employed and the route of administration utilized. The dose ratio between toxic and therapeutic effects is the therapeutic index and can be expressed as the ratio LD₅₀/ED₅₀. Compositions and methods that exhibit large therapeutic indices are preferred. A therapeutically effective dose can be estimated initially from cell culture assays. Also, a dose can be formulated in animal models to achieve a circulating plasma concentration range that includes the IC₅₀ (i.e., the concentration of the antifungal and one or more potentiator compounds), which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay. The dosage can be determined by a physician and adjusted, as necessary, to suit observed effects of the treatment.

For example, in some embodiments of the aspects described herein, a given potentiator compound, including, for example, a variant of the potentiator compounds described herein, is tested for toxicity effects in vivo. For example, single and multiple dose protocols are contemplated for assessing the toxicity to mammals of the potentiator compounds. For the single administration protocol, the inhibitors are administered intravenously, intraperitoneally or subcutaneously to mice at doses ranging from 0 to 1000 mg/kg. The 50% lethal dose (LD50) is calculated based on the mortality rate observed seven days after inhibitor administration. For the multiple administration protocol, the inhibitors are administered intravenously, intraperitoneally or subcutaneously to mice once daily for seven consecutive days at doses ranging from 0 to 1000 mg/kg. The LD50 is calculated based on the mortality rate observed seven days after the final inhibitor administration.

Depending on the type and severity of the infection, about 1 μg/kg to 100 mg/kg (e.g., 0.1-20 mg/kg) of a potentiator compound is an initial candidate dosage range for administration to the subject, whether, for example, by one or more separate administrations, or by continuous infusion. A typical daily dosage might range from about 1 μg/kg to about 100 mg/kg or more, depending on the factors mentioned above. For repeated administrations over several days or longer, depending on the condition, the treatment is sustained until the infection is treated or cleared, as measured by the methods described above or known in the art. However, other dosage regimens may be useful. The progress of the therapeutic methods described herein is easily monitored by conventional techniques and assays, such as those described herein, or known to one of skill in the art.

The duration of the therapeutic methods described herein can continue for as long as medically indicated or until a desired therapeutic effect (e.g., those described herein) is achieved. In certain embodiments, administration of a combination of an antifungal agent and one or more potentiator compounds is continued for at least 1 month, at least 2 months, at least 4 months, at least 6 months, at least 8 months, at least 10 months, at least 1 year, at least 2 years, at least 3 years, at least 4 years, at least 5 years, at least 10 years, at least 20 years, or for at least a period of years up to the lifetime of the subject. In those embodiments of the methods described herein relating to chronic infections or biofilm formation, administration is continued for as long as an in-dwelling device is present in the subject.

The potentiator compounds and antifungal agents described herein, can be administered, individually, but concurrently, in some embodiments, or, in other embodiments, simultaneously, for example in a single formulation comprising both an antifungal agent and one or more potentiator compounds, to a subject, e.g., a human subject, in accordance with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. Exemplary modes of administration of the antifungal and potentiator compounds, include, but are not limited to, injection, infusion, inhalation (e.g., intranasal or intratracheal), ingestion, rectal, and topical (including buccal and sublingual) administration. Local administration can be used if, for example, extensive side effects or toxicity is associated with the antifungal agent and/or potentiator compound, and to, for example, permit a high localized concentration of the potentiator compound to the infection site. An ex vivo strategy can also be used for therapeutic applications. Accordingly, any mode of administration that delivers the potentiator compound with/without the antifungal agent compounds systemically or to a desired surface or target, and can include, but is not limited to, injection, infusion, instillation, and inhalation administration. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “parenteral administration” and “administered parenterally” as used herein, refer to modes of administration other than enteral and topical administration, usually by injection. The phrases “systemic administration,” “administered systemically”, “peripheral administration” and “administered peripherally” as used herein refer to the administration of an antifungal agent and potentiator compounds other than directly into a target site, tissue, or organ, such as the lung, such that it enters the subject's circulatory system and, thus, is subject to metabolism and other like processes.

The type of antifungal being used to treat an infection or inhibit biofilm formation in a subject can determine the mode of administration to be used.

### Pharmaceutical Formulations

Therapeutic formulations of one or more potentiator compounds with/without an antifungal agent can be prepared, in some aspects, by mixing an antifungal agent and/or Potentiator compound having the desired degree of purity with one or more pharmaceutically acceptable carriers, excipients or stabilizers (Remington's Pharmaceutical Sciences 16th edition, Osol, A. Ed. (1980)), in the form of lyophilized formulations or aqueous solutions, either individually in some embodiments, or in combination, e.g., a therapeutic formulation comprising alone an effective amount of an antifungal agent and an effective amount of one or more Potentiator compounds. Such therapeutic formulations of the antifungals and/or potentiator compounds described herein include formulation into pharmaceutical compositions or pharmaceutical formulations for parenteral administration, e.g., intravenous; mucosal, e.g., intranasal; enteral, e.g., oral; topical, e.g., transdermal; ocular, or other mode of administration.

In one aspect, described herein is a potentiator compound for use in inhibiting or treating a fungal infection, wherein the potentiator compound is an agonist of the RAS/PKA pathway; an agonist of the TCA cycle or respiration; an inhibitor of double strand break repair, cAMP or a mimetic or analog thereof; a cAMP modulator, a phosphodiesterase inhibitor, or glucose. In some embodiments, the compound can be coformulated with an antifungal agent.

In some embodiments, provided herein is a composition comprising an antifungal agent formulated in a glucose solution.

As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent, media, encapsulating material, manufacturing aid (e.g., lubricant, talc magnesium, calcium or zinc stearate, or steric acid), or solvent encapsulating material, involved in maintaining the activity of, carrying, or transporting the antifungals and/or Potentiator compounds, from one organ, or portion of the body, to another organ, or portion of the body.

Some non-limiting examples of acceptable carriers, excipients, or stabilizers that are nontoxic to recipients at the dosages and concentrations employed, include pH buffered solutions such as phosphate, citrate, and other organic acids; antioxidants, including ascorbic acid and methionine; lubricating agents, such as magnesium stearate, sodium lauryl sulfate and talc; excipients, such as cocoa butter and suppository waxes; oils, such as peanut oil, cottonseed oil, safflower oil, sesame oil, olive oil, corn oil and soybean oil; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, HDL, LDL, or immunoglobulins; hydrophilic polymers, such as polyvinylpyrrolidone; amino acids such as glycine, glutamine, asparagine, histidine, arginine, or lysine; monosaccharides, disaccharides, and other carbohydrates including mannose, starches (corn starch or potato starch), or dextrins; cellulose, and its derivatives, such as sodium carboxymethyl cellulose, methylcellulose, ethyl cellulose, microcrystalline cellulose and cellulose acetate; chelating agents such as EDTA; sugars such as sucrose, glucose, lactose, mannitol, trehalose or sorbitol; salt-forming counter-ions such as sodium; metal complexes (e.g. Zn-protein complexes); glycols, such as propylene glycol; polyols, such as glycerin; esters, such as ethyl oleate and ethyl laurate; agar, buffering agents, such as magnesium hydroxide and aluminum hydroxide; alginic acid; pyrogen-free water, isotonic saline; Ringer's solution; polyesters, polycarbonates and/or polyanhydrides; C2-C12 alcohols, such as ethanol; powdered tragacanth; malt; and/or non-ionic surfactants such as TWEEN™, PLURONICS™ or polyethylene glycol (PEG); and/or other non-toxic compatible substances employed in pharmaceutical formulations. Wetting agents, coloring agents, release agents, coating agents, sweetening agents, flavoring agents, perfuming agents, preservative and antioxidants can also be present in the formulation.

In some embodiments, therapeutic formulations or compositions comprising an antifungal agent and/or potentiator compound comprises a pharmaceutically acceptable salt, typically, e.g., sodium chloride, and preferably at about physiological concentrations. Optionally, the formulations described herein can contain a pharmaceutically acceptable preservative. In some embodiments, the preservative concentration ranges from 0.1 to 2.0%, typically v/v. Suitable preservatives include those known in the pharmaceutical arts. Benzyl alcohol, phenol, m-cresol, methylparaben, and propylparaben are examples of preservatives. Optionally, the formulations of the invention can include a pharmaceutically acceptable surfactant at a concentration of 0.005 to 0.02%.

In some embodiments of the aspects described herein, an antifungal agent and/or potentiator compound, can be specially formulated for administration of the compound to a subject in solid, liquid or gel form, including those adapted for the following: (1) parenteral administration, for example, by subcutaneous, intramuscular, intravenous or epidural injection as, for example, a sterile solution or suspension, or sustained-release formulation; (2) topical application, for example, as a cream, ointment, or a controlled-release patch or spray applied to the skin; (3) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (4) intravaginally or intrarectally, for example, as a pessary, cream or foam; (5) sublingually; (6) ocularly; (7) transdermally; (8) transmucosally; or (9) nasally. Additionally, an antifungal agent and/or potentiator compound, can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960. Examples of dosage forms include, but are not limited to: tablets; caplets; capsules, such as hard gelatin capsules and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquids such as suspensions (e.g., aqueous or non-aqueous liquid suspensions, oil-in-water emulsions, or water-in-oil liquid emulsions), solutions, and elixirs; and sterile solids (e.g., crystalline or amorphous solids) that can be reconstituted to provide liquid dosage forms.

In some embodiments of the compositions and methods described herein, parenteral dosage forms of the compositions comprising an antifungal agent and/or potentiator compound, can be administered to a subject with a fungal infection or at risk for fungal infection by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

Suitable vehicles that can be used to provide parenteral dosage forms described herein are well known to those skilled in the art. Examples of such vehicles include, without limitation: sterile water, water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Topical dosage forms of the potentiator compounds and/or antifungal agents, are also provided in some embodiments, and include, but are not limited to, creams, lotions, ointments, gels, shampoos, sprays, aerosols, solutions, emulsions, and other forms known to one of skill in the art. See, e.g., Remington's Pharmaceutical Sciences, 18th ed., Mack Publishing, Easton, Pa. (1990); and Introduction to Pharmaceutical Dosage Forms, 4th ed., Lea & Febiger, Philadelphia, Pa. (1985). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon), or in a squeeze bottle. Moisturizers or humectants can also be added to pharmaceutical compositions and dosage forms if desired. Examples of such additional ingredients are well known in the art. See, e.g., Remington's Pharmaceutical Sciences, 18.sup.th Ed., Mack Publishing, Easton, Pa. (1990). and Introduction to Pharmaceutical Dosage Forms, 4th Ed., Lea & Febiger, Philadelphia, Pa. (1985). Dosage forms suitable for treating mucosal tissues within the oral cavity can be formulated as mouthwashes, as oral gels, or as buccal patches. Additional transdermal dosage forms include “reservoir type” or “matrix type” patches, which can be applied to the skin and worn for a specific period of time to permit the penetration of a desired amount of active ingredient.

Examples of transdermal dosage forms and methods of administration that can be used to administer one or more potentiator compounds and/or antifungal agent, include, but are not limited to, those disclosed in U.S. Pat. Nos. 4,624,665; 4,655,767; 4,687,481; 4,797,284; 4,810,499; 4,834,978; 4,877,618; 4,880,633; 4,917,895; 4,927,687; 4,956,171; 5,035,894; 5,091,186; 5,163,899; 5,232,702; 5,234,690; 5,273,755; 5,273,756; 5,308,625; 5,356,632; 5,358,715; 5,372,579; 5,421,816; 5,466;465; 5,494,680; 5,505,958; 5,554,381; 5,560,922; 5,585,111; 5,656,285; 5,667,798; 5,698,217; 5,741,511; 5,747,783; 5,770,219; 5,814,599; 5,817,332; 5,833,647; 5,879,322; and 5,906,830, each of which are incorporated herein by reference in their entirety.

Suitable excipients (e.g., carriers and diluents) and other materials that can be used to provide transdermal and mucosal dosage forms of the potentiator compounds and/or antifungal agents described herein are well known to those skilled in the pharmaceutical arts, and depend on the particular tissue or organ to which a given pharmaceutical composition or dosage form will be applied. In addition, depending on the specific tissue to be treated, additional components may be used prior to, in conjunction with, or subsequent to treatment with a potentiator compound and/or antifungal agent. For example, penetration enhancers can be used to assist in delivering the active ingredients to or across the tissue.

In some embodiments, the compositions comprising an effective amount of one or more potentiator compounds and/or an effective amount of an antifungal agent, are formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington's Pharmaceutical Sciences, 18th ed., Mack Publishing, Easton, Pa. (1990).

Due to their ease of administration, tablets and capsules represent the most advantageous solid oral dosage unit forms, in which case solid pharmaceutical excipients are used. If desired, tablets can be coated by standard aqueous or nonaqueous techniques. These dosage forms can be prepared by any of the methods of pharmacy. In general, pharmaceutical compositions and dosage forms are prepared by uniformly and intimately admixing the active ingredient(s) with liquid carriers, finely divided solid carriers, or both, and then shaping the product into the desired presentation if necessary. In some embodiments, oral dosage forms are not used for the antifungal agent.

Typical oral dosage forms of the compositions an effective amount of one or more potentiator compounds and/or an effective amount of an antifungal agent are prepared by combining the pharmaceutically acceptable salt of the one or more potentiator compounds and/or the antifungal agent, in an intimate admixture with at least one excipient according to conventional pharmaceutical compounding techniques. Excipients can take a wide variety of forms depending on the form of the composition desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, microcrystalline cellulose, kaolin, diluents, granulating agents, lubricants, binders, and disintegrating agents.

Binders suitable for use in the pharmaceutical formulations described herein include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Examples of fillers suitable for use in the pharmaceutical formulations described herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions described herein is typically present in from about 50 to about 99 weight percent of the pharmaceutical composition.

Disintegrants are used in the oral pharmaceutical formulations described herein to provide tablets that disintegrate when exposed to an aqueous environment. A sufficient amount of disintegrant that is neither too little nor too much to detrimentally alter the release of the active ingredient(s) should be used to form solid oral dosage forms of the one or more potentiator compounds and/or the antifungal agent described herein. The amount of disintegrant used varies based upon the type of formulation, and is readily discernible to those of ordinary skill in the art. Disintegrants that can be used to form oral pharmaceutical formulations include, but are not limited to, agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums, and mixtures thereof.

Lubricants that can be used to form oral pharmaceutical formulations of the one or more potentiator compounds and/or the antifungal agent described herein, include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL® 200, manufactured by W. R. Grace Co. of Baltimore, Md.), a coagulated aerosol of synthetic silica (marketed by Degussa Co. of Piano, Tex.), CAB-O-SIL® (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

In other embodiments, lactose-free pharmaceutical formulations and dosage forms are provided, wherein such compositions preferably contain little, if any, lactose or other mono- or di-saccharides. As used herein, the term “lactose-free” means that the amount of lactose present, if any, is insufficient to substantially increase the degradation rate of an active ingredient. Lactose-free compositions of the disclosure can comprise excipients which are well known in the art and are listed in the USP (XXI)/NF (XVI), which is incorporated herein by reference.

The oral formulations of the one or more potentiator compounds and/or the antifungal agent, further encompass, in some embodiments, anhydrous pharmaceutical compositions and dosage forms comprising the one or more potentiator compounds and/or the antifungal agent described herein as active ingredients, since water can facilitate the degradation of some compounds. For example, the addition of water (e.g., 5%) is widely accepted in the pharmaceutical arts as a means of simulating long-term storage in order to determine characteristics such as shelf life or the stability of formulations over time. See, e.g., Jens T. Carstensen, Drug Stability: Principles & Practice, 379-80 (2nd ed., Marcel Dekker, N.Y., N.Y.: 1995). Anhydrous pharmaceutical compositions and dosage forms described herein can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected. Anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials) with or without desiccants, blister packs, and strip packs.

One or more potentiator compounds and/or an antifungal agent can, in some embodiments of the methods described herein, be administered directly to the airways in the form of an aerosol or by nebulization. Accordingly, for use as aerosols, in some embodiments, one or more potentiator compounds and/or an antifungal agent, can be packaged in a pressurized aerosol container together with suitable propellants, for example, hydrocarbon propellants like propane, butane, or isobutane with conventional adjuvants. In other embodiments, the one or more potentiator compounds and/or the antifungal agent can be administered in a non-pressurized form such as in a nebulizer or atomizer.

The term “nebulization” is well known in the art to include reducing liquid to a fine spray. Preferably, by such nebulization small liquid droplets of uniform size are produced from a larger body of liquid in a controlled manner. Nebulization can be achieved by any suitable means, including by using many nebulizers known and marketed today. As is well known, any suitable gas can be used to apply pressure during the nebulization, with preferred gases being those which are chemically inert to the one or more potentiator compounds and/or the antifungal agent described herein. Exemplary gases include, but are not limited to, nitrogen, argon or helium.

In other embodiments, one or more potentiator compounds and/or an antifungal agent, can be administered directly to the airways in the form of a dry powder. For use as a dry powder, the one or more potentiator compounds and/or the antifungal agent can be administered by use of an inhaler. Exemplary inhalers include metered dose inhalers and dry powdered inhalers.

Suitable powder compositions include, by way of illustration, powdered preparations of one or more potentiator compounds and/or the antifungal agent, thoroughly intermixed with lactose, or other inert powders acceptable for, e.g., intrabronchial administration. The powder compositions can be administered via an aerosol dispenser or encased in a breakable capsule which may be inserted by the subject into a device that punctures the capsule and blows the powder out in a steady stream suitable for inhalation. The compositions can include propellants, surfactants, and co-solvents and may be filled into conventional aerosol containers that are closed by a suitable metering valve.

Aerosols for the delivery to the respiratory tract are known in the art. See for example, Adjei, A. and Garren, J. Pharm. Res., 1: 565-569 (1990); Zanen, P. and Lamm, J.-W. J. Int. J. Pharm., 114: 111-115 (1995); Gonda, I. “Aerosols for delivery of therapeutic and diagnostic agents to the respiratory tract,” in Critical Reviews in Therapeutic Drug Carrier Systems, 6:273-313 (1990); Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)); Timsina et. al., Int. J. Pharm., 101: 1-13 (1995); and Tansey, I. P., Spray Technol. Market, 4:26-29 (1994); French, D. L., Edwards, D. A. and Niven, R. W., Aerosol Sci., 27: 769-783 (1996); Visser, J., Powder Technology 58: 1-10 (1989)); Rudt, S. and R. H. Muller, J. Controlled Release, 22: 263-272 (1992); Tabata, Y, and Y. Ikada, Biomed. Mater. Res., 22: 837-858 (1988); Wall, D. A., Drug Delivery, 2: 10 1-20 1995); Patton, J. and Platz, R., Adv. Drug Del. Rev., 8: 179-196 (1992); Bryon, P., Adv. Drug. Del. Rev., 5: 107-132 (1990); Patton, J. S., et al., Controlled Release, 28: 15 79-85 (1994); Damms, B. and Bains, W., Nature Biotechnology (1996); Niven, R. W., et al., Pharm. Res., 12(9); 1343-1349 (1995); and Kobayashi, S., et al., Pharm. Res., 13(1): 80-83 (1996), contents of all of which are herein incorporated by reference in their entirety.

In some embodiments, the active ingredients of the formulations comprising the one or more potentiator compounds and/or the antifungal agent described herein, can also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

In some embodiments of these aspects, the one or more potentiator compounds and/or the antifungal agent, can be administered to a subject by controlled- or delayed-release means. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, Controlled Release Dosage Form Design, 2 (Technomic Publishing, Lancaster, Pa.: 2000)). Controlled-release formulations can be used to control, for example, an antifungal agent's onset of action, duration of action, plasma levels within the therapeutic window, and peak blood levels. In particular, controlled- or extended-release dosage forms or formulations can be used to ensure that the maximum effectiveness of the one or more potentiator compounds and/or the antifungal agent, is achieved while minimizing potential adverse effects and safety concerns, which can occur both from under-dosing a drug (i.e., going below the minimum therapeutic levels) as well as exceeding the toxicity level for the drug.

A variety of known controlled- or extended-release dosage forms, formulations, and devices can be adapted for use with the compositions comprising one or more potentiator compounds with/without the antifungal agent described herein Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated ins entirety herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, DUOLITE® A568 and DUOLITE® AP143 (Rohm&Haas, Spring House, Pa. USA).

In some embodiments of the aspects, the one or more potentiator compounds with/without the antifungal agent for use in the various therapeutic formulations and compositions, and methods thereof described herein, are administered to a subject by sustained release or in pulses. Pulse therapy is not a form of discontinuous administration of the same amount of a composition over time, but comprises administration of the same dose of the composition at a reduced frequency or administration of reduced doses. Sustained release or pulse administrations are particularly preferred in chronic fungal conditions, as each pulse dose can be reduced and the total amount of a compound, such as, for example, an antifungal agent, administered over the course of treatment to the patient is minimized.

The interval between pulses, when necessary, can be determined by one of ordinary skill in the art. Often, the interval between pulses can be calculated by administering another dose of the composition when the composition or the active component of the composition is no longer detectable in the subject prior to delivery of the next pulse. Intervals can also be calculated from the in vivo half-life of the composition. Intervals may be calculated as greater than the in vivo half-life, or 2, 3, 4, 5 and even 10 times greater the composition half-life. Various methods and apparatus for pulsing compositions by infusion or other forms of delivery to the patient are disclosed in U.S. Pat. Nos. 4,747,825; 4,723,958; 4,948,592; 4,965,251 and 5,403,590.

In some embodiments, sustained-release preparations comprising the one or more potentiator compounds with/without the antifungal agent, can be prepared. Suitable examples of sustained-release preparations include semipermeable matrices of solid hydrophobic polymers containing the inhibitor, in which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT (injectable microspheres composed of lactic acid-glycolic acid copolymer and leuprolide acetate), and poly-D-(−)-3-hydroxybutyric acid.

The formulations comprising the one or more potentiator compounds with/without the antifungal agent described herein, to be used for in vivo administration are preferably sterile. This is readily accomplished by filtration through, for example, sterile filtration membranes, and other methods known to one of skill in the art.

### DEFINITIONS

For convenience, certain terms employed herein, in the specification, examples and appended claims are collected here. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

Other than in the operating examples, or where otherwise indicated, all numbers expressing quantities of ingredients or reaction conditions used herein should be understood as modified in all instances by the term “about.” In various embodiments, the term “about” when used in connection with percentages means ±10, ±5, or, ±1%.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA.

“G,” “C,” “A,” “T” and “U” each generally stand for a nucleotide that contains guanine, cytosine, adenine, thymidine and uracil as a base, respectively. However, it will be understood that the term “ribonucleotide” or “nucleotide” can also refer to a modified nucleotide, as further detailed below, or a surrogate replacement moiety. The skilled person is well aware that guanine, cytosine, adenine, and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of inhibitory nucleic acids featured in the invention by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured in the invention.

As used herein, “target sequence” refers to a contiguous portion of the nucleotide sequence of an mRNA molecule formed during the transcription of a target gene, including messenger RNA (mRNA) that is a product of RNA processing of a primary transcription product. The target portion of the sequence will be at least long enough to serve as a substrate for iRNA-directed cleavage at or near that portion. For example, the target sequence will generally be from 9-36 nucleotides in length, e.g., 15-30 nucleotides in length, including all sub-ranges therebetween. As non-limiting examples, the target sequence can be from 15-30 nucleotides, 15-26 nucleotides, 15-23 nucleotides, 15-22 nucleotides, 15-21 nucleotides, 15-20 nucleotides, 15-19 nucleotides, 15-18 nucleotides, 15-17 nucleotides, 18-30 nucleotides, 18-26 nucleotides, 18-23 nucleotides, 18-22 nucleotides, 18-21 nucleotides, 18-20 nucleotides, 19-30 nucleotides, 19-26 nucleotides, 19-23 nucleotides, 19-22 nucleotides, 19-21 nucleotides, 19-20 nucleotides, 20-30 nucleotides, 20-26 nucleotides, 20-25 nucleotides, 20-24 nucleotides, 20-23 nucleotides, 20-22 nucleotides, 20-21 nucleotides, 21-30 nucleotides, 21-26 nucleotides, 21-25 nucleotides, 21-24 nucleotides, 21-23 nucleotides, or 21-22 nucleotides.

As used herein, the term “strand comprising a sequence” refers to an oligonucleotide comprising a chain of nucleotides that is described by the sequence referred to using the standard nucleotide nomenclature.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions may include: 400 mM NaCl, 40 mM PIPES pH 6.4, 1 mM EDTA, 50° C. or 70° C. for 12-16 hours followed by washing. Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides.

Complementary sequences within an iRNA, e.g., within a dsRNA as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they can form one or more, but generally not more than 5, 4, 3 or 2 mismatched base pairs upon hybridization for a duplex up to 30 base pairs (bp), while retaining the ability to hybridize under the conditions most relevant to their ultimate application, e.g., inhibition of gene expression via a RISC pathway. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, can yet be referred to as “fully complementary” for the purposes described herein.

“Complementary” sequences, as used herein, can also include, or be formed entirely from, non-Watson-Crick base pairs and/or base pairs formed from non-natural and modified nucleotides, in as far as the above requirements with respect to their ability to hybridize are fulfilled. Such non-Watson-Crick base pairs includes, but are not limited to, G:U Wobble or Hoogstein base pairing.

The terms “complementary,” “fully complementary” and “substantially complementary” herein can be used with respect to the base matching between the sense strand and the antisense strand of a dsRNA, or between the antisense strand of an iRNA agent and a target sequence, as will be understood from the context of their use.

As used herein, a polynucleotide that is “substantially complementary to at least part of” a messenger RNA (an mRNA) refers to a polynucleotide that is substantially complementary to a contiguous portion of the mRNA of interest (e.g., an mRNA encoding a target described herein). For example, a polynucleotide is complementary to at least a part of a target mRNA if the sequence is substantially complementary to a non-interrupted portion of an mRNA encoding the target.

The term “double-stranded RNA” or “dsRNA,” as used herein, refers to an iRNA that includes an RNA molecule or complex of molecules having a hybridized duplex region that comprises two anti-parallel and substantially complementary nucleic acid strands, which will be referred to as having “sense” and “antisense” orientations with respect to a target RNA. The duplex region can be of any length that permits specific degradation of a desired target RNA through a RISC pathway, but will typically range from 9 to 36 base pairs in length, e.g., 15-30 base pairs in length. Considering a duplex between 9 and 36 base pairs, the duplex can be any length in this range, for example, 9, 10, 11, 12, 13, 14, 15, 16, 17, 18, 19, 20, 21, 22, 23, 24, 25, 26, 27, 28, 29, 30, 31, 32, 33, 34, 35, or 36 and any sub-range therein between, including, but not limited to 15-30 base pairs, 15-26 base pairs, 15-23 base pairs, 15-22 base pairs, 15-21 base pairs, 15-20 base pairs, 15-19 base pairs, 15-18 base pairs, 15-17 base pairs, 18-30 base pairs, 18-26 base pairs, 18-23 base pairs, 18-22 base pairs, 18-21 base pairs, 18-20 base pairs, 19-30 base pairs, 19-26 base pairs, 19-23 base pairs, 19-22 base pairs, 19-21 base pairs, 19-20 base pairs, 20-30 base pairs, 20-26 base pairs, 20-25 base pairs, 20-24 base pairs, 20-23 base pairs, 20-22 base pairs, 20-21 base pairs, 21-30 base pairs, 21-26 base pairs, 21-25 base pairs, 21-24 base pairs, 21-23 base pairs, or 21-22 base pairs. dsRNAs generated in the cell by processing with Dicer and similar enzymes are generally in the range of 19-22 base pairs in length. One strand of the duplex region of a dsDNA comprises a sequence that is substantially complementary to a region of a target RNA. The two strands forming the duplex structure can be from a single RNA molecule having at least one self-complementary region, or can be formed from two or more separate RNA molecules. Where the duplex region is formed from two strands of a single molecule, the molecule can have a duplex region separated by a single stranded chain of nucleotides (herein referred to as a “hairpin loop”) between the 3′-end of one strand and the 5′-end of the respective other strand forming the duplex structure. The hairpin loop can comprise at least one unpaired nucleotide; in some embodiments the hairpin loop can comprise at least 3, at least 4, at least 5, at least 6, at least 7, at least 8, at least 9, at least 10, at least 20, at least 23 or more unpaired nucleotides. Where the two substantially complementary strands of a dsRNA are comprised by separate RNA molecules, those molecules need not, but can be covalently connected. Where the two strands are connected covalently by means other than a hairpin loop, the connecting structure is referred to as a “linker.” The term “siRNA” is also used herein to refer to a dsRNA as described above.

The skilled artisan will recognize that the term “RNA molecule” or “ribonucleic acid molecule” encompasses not only RNA molecules as expressed or found in nature, but also analogs and derivatives of RNA comprising one or more ribonucleotide/ribonucleoside analogs or derivatives as described herein or as known in the art. Strictly speaking, a “ribonucleoside” includes a nucleoside base and a ribose sugar, and a “ribonucleotide” is a ribonucleoside with one, two or three phosphate moieties. However, the terms “ribonucleoside” and “ribonucleotide” can be considered to be equivalent as used herein. The RNA can be modified in the nucleobase structure or in the ribose-phosphate backbone structure, e.g., as described herein below. However, the molecules comprising ribonucleoside analogs or derivatives must retain the ability to form a hybridized duplex with a complementary nucleic acid. As non-limiting examples, an RNA molecule can also include at least one modified ribonucleoside including but not limited to a 2′-O-methyl modified nucleoside, a nucleoside comprising a 5′ phosphorothioate group, a terminal nucleoside linked to a cholesteryl derivative or dodecanoic acid bisdecylamide group, a locked nucleoside, an abasic nucleoside, a 2′-deoxy-2′-fluoro modified nucleoside, a 2′-amino-modified nucleoside, 2′-alkyl-modified nucleoside, morpholino nucleoside, a phosphoramidate or a non-natural base comprising nucleoside, or any combination thereof. Alternatively, an RNA molecule can comprise at least two modified ribonucleosides, at least 3, at least 4, at least 5, at least 6, at least 7, at least 8, at least 9, at least 10, at least 15, at least 20 or more, up to the entire length of the dsRNA molecule. The modifications need not be the same for each of such a plurality of modified ribonucleosides in an RNA molecule. In one embodiment, modified RNAs contemplated for use in methods and compositions described herein are peptide nucleic acids (PNAs) that have the ability to form the required duplex structure and that permit or mediate the specific degradation of a target RNA via a RISC pathway.

Modification of an RNA or dsRNA can improve not only stability, but also the tolerance of the dsRNA by the subject to which it is delivered. It is known in the art that dsRNA can provoke a cellular stress response related to the body's material defense against pathogens such as viruses. The so-called “interferon response” or “PKR response” (for involvement of protein kinase R) is triggered to some extent by exogenous RNA in general, and particularly by dsRNA greater than about 30 nucleotides in length. While limiting dsRNAs to less than 30 nucleotides will avoid a significant portion of the stress response, even shorter exogenous RNAs, and particularly dsRNA can provoke some degree of stress response in mammals. This response has a component that is sequence-specific, in that certain sequence motifs will or will not provoke the response, and the response can be exacerbated with repeated administration. RNA modification or sequence selection strategies for further minimizing the stress response so as to optimize the desired effects and permit repeated administration without loss of activity are described, for example, in U.S. 20120045461, U.S. 20090169529 and WO 2011/130624, each of which is incorporated herein in its entirety by reference.

In one aspect, a modified ribonucleoside includes a deoxyribonucleoside. In such an instance, an iRNA agent can comprise one or more deoxynucleosides, including, for example, a deoxynucleoside overhang(s), or one or more deoxynucleosides within the double stranded portion of a dsRNA. However, it is self evident that under no circumstances is a double stranded DNA molecule encompassed by the term “iRNA.”

The term “antisense strand” or “guide strand” refers to the strand of an iRNA, e.g., a dsRNA, which includes a region that is substantially complementary to a target sequence. As used herein, the term “region of complementarity” refers to the region on the antisense strand that is substantially complementary to a sequence, for example a target sequence, as defined herein. Where the region of complementarity is not fully complementary to the target sequence, the mismatches may be in the internal or terminal regions of the molecule. Generally, the most tolerated mismatches are in the terminal regions, e.g., within 5, 4, 3, or 2 nucleotides of the 5′ and/or 3′ terminus.

The term “sense strand,” or “passenger strand” as used herein, refers to the strand of an iRNA that includes a region that is substantially complementary to a region of the antisense strand as that term is defined herein.

As used herein, the term “RNAi” refers to any type of interfering RNA, including but not limited to RNAi, siRNA, shRNA, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the generation of active cleavage complexes and the site-specific cleavage of mRNA, such sequences can be incorporated into vectors for direct expression or used for direct introduction to cells). The term “RNAi” and “RNA interference” with respect to an agent of the technology described herein, are used interchangeably herein.

As used herein a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA can be formed from separate complementary strands. In one embodiment, a siRNA refers to a nucleic acid that can form a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA formed from a single, at least partially self-complementary strand of RNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow. The double-stranded portion that forms upon intramolecular hybridization of the sense and antisense sequences corresponds to the targeted mRNA sequence.

As used herein, the terms “protein” and “polypeptide” are used interchangeably to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a translated gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein an “antibody” refers to IgG, IgM, IgA, IgD or IgE molecules or antigen-specific antibody fragments thereof (including, but not limited to, a Fab, F(ab)₂, Fv, disulphide linked Fv, scFv, single domain antibody, closed conformation multispecific antibody, disulphide-linked scfv, diabody), whether derived from any species that naturally produces an antibody, or created by recombinant DNA technology; whether isolated from serum, B-cells, hybridomas, transfectomas, yeast or bacteria.

As described herein, an “antigen” is a molecule that is bound by a binding site on an antibody agent. Typically, antigens are bound by antibody ligands and are capable of raising an antibody response in vivo. An antigen can be a polypeptide, protein, nucleic acid or other molecule or portion thereof. The term “antigenic determinant” refers to an epitope on the antigen recognized by an antigen-binding molecule, and more particularly, by the antigen-binding site of said molecule.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, and domain antibodies (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, IgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like.

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties). Each VH and VL is typically composed of three CDRs and four FRs, arranged from amino-terminus to carboxy-terminus in the following order: FR1, CDR1, FR2, CDR2, FR3, CDR3, FR4.

The terms “antigen-binding fragment” or “antigen-binding domain”, which are used interchangeably herein are used to refer to one or more fragments of a full length antibody that retain the ability to specifically bind to a target of interest. Examples of binding fragments encompassed within the term “antigen-binding fragment” of a full length antibody include (i) a Fab fragment, a monovalent fragment consisting of the VL, VH, CL and CH1 domains; (ii) a F(ab′)2 fragment, a bivalent fragment including two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment (Ward et al., (1989) Nature 341:544-546; which is incorporated by reference herein in its entirety), which consists of a VH or VL domain; and (vi) an isolated complementarity determining region (CDR) that retains specific antigen-binding functionality. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv). See e.g., U.S. Pat. Nos. 5,260,203, 4,946,778, and 4,881,175; Bird et al. (1988) Science 242:423-426; and Huston et al. (1988) Proc. Natl. Acad. Sci. USA 85:5879-5883. Antibody fragments can be obtained using any appropriate technique including conventional techniques known to those of skill in the art. The term “monospecific antibody” refers to an antibody that displays a single binding specificity and affinity for a particular target, e.g., epitope. This term includes a “monoclonal antibody” or “monoclonal antibody composition,” which as used herein refer to a preparation of antibodies or fragments thereof of single molecular composition, irrespective of how the antibody was generated.

A further kind of antibody reagent is an intrabody i.e. an intracellular antibody (See, generally, Hood et al., Immunology, Benjamin, N.Y., 2ND ed. (1984), Harlow and Lane, Antibodies. A Laboratory Manual, Cold Spring Harbor Laboratory (1988) and Hunkapiller and Hood, Nature, 323, 15-16 (1986), which are incorporated herein by reference). Intrabodies work within the cell and bind intracellular protein. Intrabodies can include whole antibodies or antibody binding fragments thereof, e.g. single Fv, Fab and F(ab)′2, etc. Methods for intrabody production are well known to those of skill in the art, e.g. as described in WO 2002/086096. Antibodies will usually bind with at least a KD of about 1 mM, more usually at least about 300 μM, typically at least about 10 μM, more typically at least about 30 μM, preferably at least about 10 μM, and more preferably at least about 3 μM or better.).

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity.

Avidity is the measure of the strength of binding between an antigen-binding molecule (such as an antibody reagent described herein) and the pertinent antigen. Avidity is related to both the affinity between an antigenic determinant and its antigen binding site on the antigen-binding molecule, and the number of pertinent binding sites present on the antigen-binding molecule. Typically, antigen-binding proteins (such as an antibody reagent described herein) will bind to their cognate or specific antigen with a dissociation constant (K_{D} of 10⁻⁵S to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles). Any K_{D} value greater than 10⁻⁴ mol/liter (or any K_{A} value lower than 10⁴ M⁻¹) is generally considered to indicate non-specific binding. The K_{D} for biological interactions which are considered meaningful (e.g. specific) are typically in the range of 10⁻¹⁰ M (0.1 nM) to 10⁻⁵ M (10000 nM). The stronger an interaction is, the lower is its K_{D}. Preferably, a binding site on an antibody reagent described herein will bind to the desired antigen with an affinity less than 500 nM, preferably less than 200 nM, more preferably less than 10 nM, such as less than 500 pM. Specific binding of an antibody reagent to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as other techniques as mentioned herein.

Accordingly, as used herein, “selectively binds” or “specifically binds” refers to the ability of an agent (e.g. an antibody reagent) described herein to bind to a target, such a peptide comprising, e.g. the amino acid sequence of a target as described herein, with a K_{D} 10⁻⁵ M (10000 nM) or less, e.g., 10⁻⁶ M or less, 10⁻⁷ M or less, 10⁻⁸ M or less, 10⁻⁹ M or less, 10⁻¹⁰M or less, 10⁻¹¹ M or less, or 10⁻¹² M or less. For example, if an agent described herein binds to a first peptide comprising a target as described herein or an epitope thereof with a K_{D} of 10⁻⁵ M or lower, but not to another randomly selected peptide, then the agent is said to specifically bind the first peptide. Specific binding can be influenced by, for example, the affinity and avidity of the agent and the concentration of the agent. The person of ordinary skill in the art can determine appropriate conditions under which an agent selectively bind the targets using any suitable methods, such as titration of an agent in a suitable cell and/or a peptide binding assay.

Traditionally, monoclonal antibodies have been produced as native molecules in murine hybridoma lines. In addition to that technology, the methods and compositions described herein provide for recombinant DNA expression of monoclonal antibodies. This allows the production of humanized antibodies as well as a spectrum of antibody derivatives and fusion proteins in a host species of choice. The production of antibodies in bacteria, yeast, transgenic animals and chicken eggs are also alternatives to hybridoma-based production systems. The main advantages of transgenic animals are potential high yields from renewable sources.

As used herein, an “epitope” can be formed both from contiguous amino acids, or noncontiguous amino acids juxtaposed by folding of a protein. Epitopes formed from contiguous amino acids are typically retained on exposure to denaturing solvents, whereas epitopes formed by folding are typically lost on treatment with denaturing solvents. An epitope typically includes at least 3, and more usually, at least 5, about 9, or about 8-10 amino acids in a unique spatial conformation. An “epitope” includes the unit of structure conventionally bound by an immunoglobulin V_{H}/V_{L} pair. Epitopes define the minimum binding site for an antibody, and thus represent the target of specificity of an antibody. In the case of a single domain antibody, an epitope represents the unit of structure bound by a variable domain in isolation. The terms “antigenic determinant” and “epitope” can also be used interchangeably herein.

Nucleic acid molecules encoding amino acid sequence variants of antibodies are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody. A nucleic acid sequence encoding at least one antibody, portion or polypeptide as described herein can be recombined with vector DNA in accordance with conventional techniques, including blunt-ended or staggered-ended termini for ligation, restriction enzyme digestion to provide appropriate termini, filling in of cohesive ends as appropriate, alkaline phosphatase treatment to avoid undesirable joining, and ligation with appropriate ligases. Techniques for such manipulations are disclosed, e.g., by Maniatis et al., Molecular Cloning, Lab. Manual (Cold Spring Harbor Lab. Press, N Y, 1982 and 1989), and Ausubel, 1987, 1993, and can be used to construct nucleic acid sequences which encode a monoclonal antibody molecule or antigen binding region thereof. A nucleic acid molecule, such as DNA, is said to be “capable of expressing” a polypeptide if it contains nucleotide sequences which contain transcriptional and translational regulatory information and such sequences are “operably linked” to nucleotide sequences which encode the polypeptide. An operable linkage is a linkage in which the regulatory DNA sequences and the DNA sequence sought to be expressed are connected in such a way as to permit gene expression as peptides or antibody portions in recoverable amounts. The precise nature of the regulatory regions needed for gene expression may vary from organism to organism, as is well known in the analogous art. See. e.g., Sambrook et al., 1989; Ausubel et al., 1987-1993.

Accordingly, the expression of an antibody or antigen-binding portion thereof as described herein can occur in either prokaryotic or eukaryotic cells. Suitable hosts include bacterial or eukaryotic hosts, including yeast, insects, fungi, bird and mammalian cells either in vivo, or in situ, or host cells of mammalian, insect, bird or yeast origin. The mammalian cell or tissue can be of human, primate, hamster, rabbit, rodent, cow, pig, sheep, horse, goat, dog or cat origin, but any other mammalian cell may be used. Further, by use of, for example, the yeast ubiquitin hydrolase system, in vivo synthesis of ubiquitin-transmembrane polypeptide fusion proteins can be accomplished. The fusion proteins so produced can be processed in vivo or purified and processed in vitro, allowing synthesis of an antibody or portion thereof as described herein with a specified amino terminus sequence. Moreover, problems associated with retention of initiation codon-derived methionine residues in direct yeast (or bacterial) expression may be avoided. Sabin et al., 7 Bio/Technol. 705 (1989); Miller et al., 7 Bio/Technol. 698 (1989). Any of a series of yeast gene expression systems incorporating promoter and termination elements from the actively expressed genes coding for glycolytic enzymes produced in large quantities when yeast are grown in media rich in glucose can be utilized to obtain recombinant antibodies or antigen-binding portions thereof. Known glycolytic genes can also provide very efficient transcriptional control signals. For example, the promoter and terminator signals of the phosphoglycerate kinase gene can be utilized.

Production of antibodies or antigen-binding portions thereof as described herein can be achieved in insects, for example, by infecting the insect host with a baculovirus engineered to express a transmembrane polypeptide by methods known to those of skill in the art. See Ausubel et al., 1987, 1993.

In some embodiments, the introduced nucleotide sequence is incorporated into a plasmid or viral vector capable of autonomous replication in the recipient host. Any of a wide variety of vectors can be employed for this purpose and are known and available to those of ordinary skill in the art. See, e.g., Ausubel et al., 1987, 1993. Factors of importance in selecting a particular plasmid or viral vector include: the ease with which recipient cells that contain the vector may be recognized and selected from those recipient cells which do not contain the vector, the number of copies of the vector which are desired in a particular host; and whether it is desirable to be able to “shuttle” the vector between host cells of different species.

Example prokaryotic vectors known in the art include plasmids such as those capable of replication in E. coli., for example. Other gene expression elements useful for the expression of cDNA encoding antibodies or antigen-binding portions thereof include, but are not limited to (a) viral transcription promoters and their enhancer elements, such as the SV40 early promoter (Okayama et al., 3 Mol. Cell. Biol. 280 (1983)), Rous sarcoma virus LTR (Gorman et al., 79 PNAS 6777 (1982)), and Moloney murine leukemia virus LTR (Grosschedl et al., 41 Cell 885 (1985)); (b) splice regions and polyadenylation sites such as those derived from the SV40 late region (Okayarea et al., 1983), and (c) polyadenylation sites such as in SV40 (Okayama et al., 1983). Immunoglobulin cDNA genes can be expressed as described by Liu et al., infra, and Weidle et al., 51 Gene 21 (1987), using as expression elements the SV40 early promoter and its enhancer, the mouse immunoglobulin H chain promoter enhancers, SV40 late region mRNA splicing, rabbit S-globin intervening sequence, immunoglobulin and rabbit S-globin polyadenylation sites, and SV40 polyadenylation elements.

For immunoglobulin genes comprised of part cDNA, part genomic DNA (Whittle et al., 1 Protein Engin. 499 (1987)), the transcriptional promoter can be human cytomegalovirus, the promoter enhancers can be cytomegalovirus and mouse/human immunoglobulin, and mRNA splicing and polyadenylation regions can be the native chromosomal immunoglobulin sequences.

In some embodiments, for expression of cDNA genes in rodent cells, the transcriptional promoter is a viral LTR sequence, the transcriptional promoter enhancers are either or both the mouse immunoglobulin heavy chain enhancer and the viral LTR enhancer, the splice region contains an intron of greater than 31 bp, and the polyadenylation and transcription termination regions are derived from the native chromosomal sequence corresponding to the immunoglobulin chain being synthesized. In other embodiments, cDNA sequences encoding other proteins are combined with the above-recited expression elements to achieve expression of the proteins in mammalian cells.

Each fused gene is assembled in, or inserted into, an expression vector. Recipient cells capable of expressing the chimeric immunoglobulin chain gene product are then transfected singly with an antibody, antigen-binding portion thereof, or chimeric H or chimeric L chain-encoding gene, or are co-transfected with a chimeric H and a chimeric L chain gene. The transfected recipient cells are cultured under conditions that permit expression of the incorporated genes and the expressed immunoglobulin chains or intact antibodies or fragments are recovered from the culture.

In some embodiments, the fused genes encoding the antibody, antigen-binding fragment thereof, or chimeric H and L chains, or portions thereof are assembled in separate expression vectors that are then used to co-transfect a recipient cell. Each vector can contain two selectable genes, a first selectable gene designed for selection in a bacterial system and a second selectable gene designed for selection in a eukaryotic system, wherein each vector has a different pair of genes. This strategy results in vectors which first direct the production, and permit amplification, of the fused genes in a bacterial system. The genes so produced and amplified in a bacterial host are subsequently used to co-transfect a eukaryotic cell, and allow selection of a co-transfected cell carrying the desired transfected genes. Non-limiting examples of selectable genes for use in a bacterial system are the gene that confers resistance to ampicillin and the gene that confers resistance to chloramphenicol. Selectable genes for use in eukaryotic transfectants include the xanthine guanine phosphoribosyl transferase gene (designated gpt) and the phosphotransferase gene from Tn5 (designated neo). Alternatively the fused genes encoding chimeric H and L chains can be assembled on the same expression vector.

For transfection of the expression vectors and production of the chimeric, humanized, or composite human antibodies described herein, the recipient cell line can be a myeloma cell. Myeloma cells can synthesize, assemble and secrete immunoglobulins encoded by transfected immunoglobulin genes and possess the mechanism for glycosylation of the immunoglobulin. For example, in some embodiments, the recipient cell is the recombinant Ig-producing myeloma cell SP2/0 (ATCC #CRL 8287). SP2/0 cells produce only immunoglobulin encoded by the transfected genes. Myeloma cells can be grown in culture or in the peritoneal cavity of a mouse, where secreted immunoglobulin can be obtained from ascites fluid. Other suitable recipient cells include lymphoid cells such as B lymphocytes of human or non-human origin, hybridoma cells of human or non-human origin, or interspecies heterohybridoma cells.

An expression vector carrying a chimeric, humanized, or composite human antibody construct, antibody, or antigen-binding portion thereof as described herein can be introduced into an appropriate host cell by any of a variety of suitable means, including such biochemical means as transformation, transfection, conjugation, protoplast fusion, calcium phosphate-precipitation, and application with polycations such as diethylaminoethyl (DEAE) dextran, and such mechanical means as electroporation, direct microinjection, and microprojectile bombardment. Johnston et al., 240 Science 1538 (1988), as known to one of ordinary skill in the art.

Yeast provides certain advantages over bacteria for the production of immunoglobulin H and L chains. Yeasts carry out post-translational peptide modifications including glycosylation. A number of recombinant DNA strategies exist that utilize strong promoter sequences and high copy number plasmids which can be used for production of the desired proteins in yeast. Yeast recognizes leader sequences of cloned mammalian gene products and secretes peptides bearing leader sequences (i.e., pre-peptides). Hitzman et al., 11th Intl. Conf. Yeast, Genetics & Molec. Biol. (Montpelier, France, 1982).

Yeast gene expression systems can be routinely evaluated for the levels of production, secretion and the stability of antibodies, and assembled chimeric, humanized, or composite human antibodies, portions and regions thereof. Any of a series of yeast gene expression systems incorporating promoter and termination elements from the actively expressed genes coding for glycolytic enzymes produced in large quantities when yeasts are grown in media rich in glucose can be utilized. Known glycolytic genes can also provide very efficient transcription control signals. For example, the promoter and terminator signals of the phosphoglycerate kinase (PGK) gene can be utilized. A number of approaches can be taken for evaluating optimal expression plasmids for the expression of cloned immunoglobulin cDNAs in yeast. See II DNA Cloning 45, (Glover, ed., IRL Press, 1985) and e.g., U.S. Publication No. US 2006/0270045 A1.

Bacterial strains can also be utilized as hosts for the production of the antibody molecules or peptides described herein, E. coli K12 strains such as E. coli W3110 (ATCC 27325), Bacillus species, enterobacteria such as Salmonella typhimurium or Serratia marcescens, and various Pseudomonas species can be used. Plasmid vectors containing replicon and control sequences which are derived from species compatible with a host cell are used in connection with these bacterial hosts. The vector carries a replication site, as well as specific genes which are capable of providing phenotypic selection in transformed cells. A number of approaches can be taken for evaluating the expression plasmids for the production of chimeric, humanized, or composite humanized antibodies and fragments thereof encoded by the cloned immunoglobulin cDNAs or CDRs in bacteria (see Glover, 1985; Ausubel, 1987, 1993; Sambrook, 1989; Colligan, 1992-1996).

Host mammalian cells can be grown in vitro or in vivo. Mammalian cells provide post-translational modifications to immunoglobulin protein molecules including leader peptide removal, folding and assembly of H and L chains, glycosylation of the antibody molecules, and secretion of functional antibody protein.

In some embodiments, one or more antibodies or antibody reagents thereof as described herein can be produced in vivo in an animal that has been engineered or transfected with one or more nucleic acid molecules encoding the polypeptides, according to any suitable method.

In some embodiments, an antibody or antibody reagent as described herein is produced in a cell-free system. Nonlimiting exemplary cell-free systems are described, e.g., in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

Many vector systems are available for the expression of cloned H and L chain genes in mammalian cells (see Glover, 1985). Different approaches can be followed to obtain complete H₂L₂ antibodies. As discussed above, it is possible to co-express H and L chains in the same cells to achieve intracellular association and linkage of H and L chains into complete tetrameric H₂L₂ antibodies or antigen-binding portions thereof. The co-expression can occur by using either the same or different plasmids in the same host. Genes for both H and L chains or portions thereof can be placed into the same plasmid, which is then transfected into cells, thereby selecting directly for cells that express both chains. Alternatively, cells can be transfected first with a plasmid encoding one chain, for example the L chain, followed by transfection of the resulting cell line with an H chain plasmid containing a second selectable marker. Cell lines producing antibodies, antigen-binding portions thereof and/or H₂L₂ molecules via either route could be transfected with plasmids encoding additional copies of peptides, H, L, or H plus L chains in conjunction with additional selectable markers to generate cell lines with enhanced properties, such as higher production of assembled H₂L₂ antibody molecules or enhanced stability of the transfected cell lines.

Additionally, plants have emerged as a convenient, safe and economical alternative main-stream expression systems for recombinant antibody production, which are based on large scale culture of microbes or animal cells. Antibodies can be expressed in plant cell culture, or plants grown conventionally. The expression in plants may be systemic, limited to susb-cellular plastids, or limited to seeds (endosperms). See, e.g., U.S. Patent Pub. No. 2003/0167531; U.S. Pat. No. 6,080,560; U.S. Pat. No. 6,512,162; WO 0129242. Several plant-derived antibodies have reached advanced stages of development, including clinical trials (see, e.g., Biolex, N.C.).

In some aspects, provided herein are methods and systems for the production of a humanized antibody, which is prepared by a process which comprises maintaining a host transformed with a first expression vector which encodes the light chain of the humanized antibody and with a second expression vector which encodes the heavy chain of the humanized antibody under such conditions that each chain is expressed and isolating the humanized antibody formed by assembly of the thus-expressed chains. The first and second expression vectors can be the same vector. Also provided herein are DNA sequences encoding the light chain or the heavy chain of the humanized antibody; an expression vector which incorporates a said DNA sequence; and a host transformed with a said expression vector.

Generating a humanized antibody from the sequences and information provided herein can be practiced by those of ordinary skill in the art without undue experimentation. In one approach, there are four general steps employed to humanize a monoclonal antibody, see. e.g., U.S. Pat. No. 5,585,089; U.S. Pat. No. 6,835,823; U.S. Pat. No. 6,824,989. These are: (1) determining the nucleotide and predicted amino acid sequence of the starting antibody light and heavy variable domains; (2) designing the humanized antibody, i.e., deciding which antibody framework region to use during the humanizing process; (3) the actual humanizing methodologies/techniques; and (4) the transfection and expression of the humanized antibody.

Usually the CDR regions in humanized antibodies and human antibody variants are substantially identical, and more usually, identical to the corresponding CDR regions in the mouse or human antibody from which they were derived. Although not usually desirable, it is sometimes possible to make one or more conservative amino acid substitutions of CDR residues without appreciably affecting the binding affinity of the resulting humanized immunoglobulin or human antibody variant. Occasionally, substitutions of CDR regions can enhance binding affinity.

In addition, techniques developed for the production of “chimeric antibodies” (see Morrison et al., Proc. Natl. Acad. Sci. 81:851-855 (1984); Neuberger et al., Nature 312:604-608 (1984); Takeda et al., Nature 314:452-454 (1985); which are incorporated by reference herein in their entireties) by splicing genes from a mouse, or other species, antibody molecule of appropriate antigen specificity together with genes from a human antibody molecule of appropriate biological activity can be used. A chimeric antibody is a molecule in which different portions are derived from different animal species, such as those having a variable region derived from a murine monoclonal antibody and a human immunoglobulin constant region, e.g., humanized antibodies. The variable segments of chimeric antibodies are typically linked to at least a portion of an immunoglobulin constant region (Fc), typically that of a human immunoglobulin. Human constant region DNA sequences can be isolated in accordance with well-known procedures from a variety of human cells, such as immortalized B-cells (WO 87/02671; which is incorporated by reference herein in its entirety). The antibody can contain both light chain and heavy chain constant regions. The heavy chain constant region can include CH1, hinge, CH2, CH3, and, sometimes, CH4 regions. For therapeutic purposes, the CH2 domain can be deleted or omitted.

Alternatively, techniques described for the production of single chain antibodies (see, e.g. U.S. Pat. No. 4,946,778; Bird, Science 242:42342 (1988); Huston et al., Proc. Natl. Acad. Sci. USA 85:5879-5883 (1988); and Ward et al., Nature 334:544-54 (1989); which are incorporated by reference herein in their entireties) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli can also be used (see, e.g. Skerra et al., Science 242:1038-1041 (1988); which is incorporated by reference herein in its entirety).

Chimeric, humanized and human antibodies are typically produced by recombinant expression. Recombinant polynucleotide constructs typically include an expression control sequence operably linked to the coding sequences of antibody chains, including naturally-associated or heterologous promoter regions. Preferably, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences, and the collection and purification of the cross-reacting antibodies. These expression vectors are typically replicable in the host organisms either as episomes or as an integral part of the host chromosomal DNA. Commonly, expression vectors contain selection markers, e.g., ampicillin-resistance or hygromycin-resistance, to permit detection of those cells transformed with the desired DNA sequences. E. coli is one prokaryotic host particularly useful for cloning the DNA sequences. Microbes, such as yeast are also useful for expression. Saccharomyces is a preferred yeast host, with suitable vectors having expression control sequences, an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization. Mammalian cells are a preferred host for expressing nucleotide segments encoding immunoglobulins or fragments thereof. See Winnacker, From Genes to Clones, (VCH Publishers, N Y, 1987), which is incorporated herein by reference in its entirety. A number of suitable host cell lines capable of secreting intact heterologous proteins have been developed in the art, and include CHO cell lines, various COS cell lines, HeLa cells, L cells and multiple myeloma cell lines. Expression vectors for these cells can include expression control sequences, such as an origin of replication, a promoter, an enhancer (Queen et al., “Cell-type Specific Regulation of a Kappa Immunoglobulin Gene by Promoter and Enhancer Elements,” Immunol Rev 89:49 (1986), incorporated herein by reference in its entirety), and necessary processing information sites, such as ribosome binding sites, RNA splice sites, polyadenylation sites, and transcriptional terminator sequences. Preferred expression control sequences are promoters substantially similar to a region of the endogenous genes, cytomegalovirus, SV40, adenovirus, bovine papillomavirus, and the like. See Co et al., “Chimeric and Humanized Antibodies with Specificity for the CD33 Antigen,” J Immunol 148:1149 (1992), which is incorporated herein by reference in its entirety. Alternatively, antibody coding sequences can be incorporated in transgenes for introduction into the genome of a transgenic animal and subsequent expression in the milk of the transgenic animal (e.g., according to methods described in U.S. Pat. No. 5,741,957, U.S. Pat. No. 5,304,489, U.S. Pat. No. 5,849,992, all incorporated by reference herein in their entireties). Suitable transgenes include coding sequences for light and/or heavy chains in operable linkage with a promoter and enhancer from a mammary gland specific gene, such as casein or beta lactoglobulin. The vectors containing the DNA segments of interest can be transferred into the host cell by well-known methods, depending on the type of cellular host. For example, calcium chloride transfection is commonly utilized for prokaryotic cells, whereas calcium phosphate treatment, electroporation, lipofection, biolistics or viral-based transfection can be used for other cellular hosts. Other methods used to transform mammalian cells include the use of polybrene, protoplast fusion, liposomes, electroporation, and microinjection (see generally, Sambrook et al., supra, which is herein incorporated by reference in is entirety). For production of transgenic animals, transgenes can be microinjected into fertilized oocytes, or can be incorporated into the genome of embryonic stem cells, and the nuclei of such cells transferred into enucleated oocytes. Once expressed, antibodies can be purified according to standard procedures of the art, including HPLC purification, column chromatography, gel electrophoresis and the like (see generally, Scopes, Protein Purification (Springer-Verlag, N Y, 1982), which is incorporated herein by reference in its entirety).

Once expressed, the whole antibodies, their dimers, individual light and heavy chains, or other immunoglobulin forms of the present invention can be recovered and purified by known techniques, e.g., immunoabsorption or immunoaffinity chromatography, chromatographic methods such as HPLC (high performance liquid chromatography), ammonium sulfate precipitation, gel electrophoresis, or any combination of these. See generally, Scopes, PROTEIN PURIF. (Springer-Verlag, N Y, 1982). Substantially pure immunoglobulins of at least about 90% to 95% homogeneity are advantageous, as are those with 98% to 99% or more homogeneity, particularly for pharmaceutical uses. Once purified, partially or to homogeneity as desired, a humanized or composite human antibody can then be used therapeutically or in developing and performing assay procedures, immunofluorescent stainings, and the like. See generally, Vols. I & II Immunol. Meth. (Lefkovits & Pernis, eds., Acad. Press, N Y, 1979 and 1981).

Additionally, and as described herein, a recombinant humanized antibody can be further optimized to decrease potential immunogenicity, while maintaining functional activity, for therapy in humans. In this regard, functional activity means a polypeptide capable of displaying one or more known functional activities associated with a recombinant antibody or antibody reagent thereof as described herein. Such functional activities include, e.g. the ability to bind to a target described herein.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology, and molecular biology can be found in The Merck Manual of Diagnosis and Therapy, 18th Edition, published by Merck Research Laboratories, 2006 (ISBN 0-911910-18-2); Robert S. Porter et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006. Definitions of common terms in molecular biology are found in Benjamin Lewin, Genes IX, published by Jones & Bartlett Publishing, 2007 (ISBN-13: 9780763740634); Kendrew et al. (eds.), The Encyclopedia of Molecular Biology, published by Blackwell Science Ltd., 1994 (ISBN 0-632-02182-9); and Robert A. Meyers (ed.), Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); or Methods in Enzymology: Guide to Molecular Cloning Techniques Vol. 152, S. L. Berger and A. R. Kimmerl Eds., Academic Press Inc., San Diego, USA (1987); Current Protocols in Molecular Biology (CPMB) (Fred M. Ausubel, et al. ed., John Wiley and Sons, Inc.), Current Protocols in Protein Science (CPPS) (John E. Coligan, et. al., ed., John Wiley and Sons, Inc.) and Current Protocols in Imnunology (CPI) (John E. Coligan, et. al., ed. John Wiley and Sons, Inc.), which are all incorporated by reference herein in their entireties.

It is understood that the following detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

This invention is further illustrated by the following examples which should not be construed as limiting. The following exemplary methods were used to demonstrate that modulating the targets described herein potentiates antifunal activity and sensitivity of fungal strains to antifungals, can be used, for example to identify additional targets and modulators thereof for use in the methods and compositions described herein.

Some embodiments of the technology described herein can be defined according to any of the following numbered paragraphs:
1. A method for inhibiting a fungal infection, the method comprising administering to a subject having or at risk for a fungal infection an effective amount of one or more potentiator compounds and an effective amount of an antifungal agent.
2. A method for inhibiting a fungal infection, the method comprising administering to a subject having or at risk for a fungal infection an effective amount of a pharmaceutical composition comprising one or more potentiator compounds and an antifungal agent.
3. A method for treating a fungal infection, comprising administering to a patient having a fungal infection and undergoing treatment with an antifungal agent, an effective amount of one or more potentiator compounds.
4. The method of any one of paragraphs 1-3, wherein the potentiator compound is an agonist of the RAS/PKA pathway; an agonist of the TCA cycle or respiration; an inhibitor of DNA repair, cAMP or a mimetic or analog thereof; a cAMP modulator, a phosphodiesterase inhibitor, or glucose.
5. The method of paragraph 4, wherein the agonist of the RAS/PKA pathway is an agonist of RAS1; RAS2; Cyr1; Cdc25; Srv2; Tpk1; Tpk2; Tpk3; and orthologs and homologs thereof; or an inhibitor of Bcy1; Pde1; Pde2; or orthologs and homologs thereof.
6. The method of paragraph 4, wherein the inhibitor of Pde1 is IC224.
7. The method of paragraph 4, wherein the agonist of the TCA cycle or respiration is an agonist of Hap2; Hap3; Hap4; Hap5; Cit1; Cit2; Sdh1/2 or orthologs and homologs thereof.
8. The method of paragraph 4, wherein the potentiator compound modulates carbon source utilization or inhibits glucose utilization.
9. The method of paragraph 4, wherein the inhibitor of DNA repair is an inhibitor of double-strand break repair; an inhibitor of single-strand repair, or an inhibitor of direct reversal.
10. The method of paragraph 4, wherein the inhibitor of double-strand break repair is an inhibitor of Rad54; Rad51; Rad52; Rad55; Rad57; RPA; Xrs2; Mre11; Lif1; Nej1; or orthologs and homologs thereof.
11. The method of paragraph 10, wherein the inhibitor is wortmannin; rapamycin; vorinostat; 0⁶-BG; NVP-BEZ235; 2-(Morpholin-4-yl)-benzo[h]chomen-4-one; 1-(2-hydroxy-4-morpholin-4-yl-phenyl)-ethanone; Ku55933; NU7441; or SU11752.
12. The method of paragraph 4, wherein the cAMP mimetic or analog or modulator thereof is diburtyryl cAMP; caffeine; forskolin; 8-bromo-cAMP; phorbol ester; sclareline; cholera toxin (CTx); aminophylline; 2,4 dinitrophenol (DNP); norepinephrine; epinephrine; isoproterenol; isobutylmethylxanthine (IBMX); theophylline (dimethylxanthine); dopamine; rolipram; iloprost; prostaglandin Et; prostaglandin E₂; pituitary adenylate cyclase activating polypeptide (PACAP); vasoactive intestinal polypeptide (VIP); (S)-adenosine; cyclic 3′,5′-(hydrogenphosphorothioate)triethyl ammonium; 8-bromoadenosine-3′,5′-cyclic monophosphate; 8-chloroadenosine-3′,5′-cyclic monophosphate; or N6,2′-O-dibutyryladenosine-3′,5′-cyclic monophosphate.
13. The method of paragraph 4, wherein the phosphodiesterase inhibitor is rolipram, mesembrine, drotaverine, roflumilast, ibudilast, piclamilast, luteolin, cilomilast, diazepam, arofylline, CP-80633, denbutylline, drotaverine, etazolate, filaminast, glaucine, HT-0712, ICI-63197, irsogladine, mesembrine, Ro20-1724, RPL-554, YM-976, sildenafil, vardenafil, tadalafil, udenafil, avanafil, sofyllin, pentoxifylline, acetildenafil, bucladesine, cilostamide, cilostazol, dipyridamole, enoximone, glaucine, ibudilast, icariin, inamrinone (formerly amrinone), lodenafil, luteolin, milrinone, mirodenafil, pimobendan, propentofylline, zardaverine, caffeine, theophylline, theobromine, 3-isobutyl-1-methylxanthine (IBMX), aminophylline, or paraxanthine.
14. The method of any one of paragraphs 1 to 13, wherein the potentiator is selected for its ability to increase ROS production or increase susceptibility to oxidative stress.
15. The method of paragraph 14, wherein the ROS is O₂⁻, H₂O₂, or O₂⁻ and H₂O₂.
16. The method of any of paragraphs 1-15, wherein the antifungal is fungicidal or fungistatic.
17. The method of any of paragraphs 1-16, wherein the antifungal agent is a polyene; an imidazole; a triazole; a thiazole; an allylamine; or an echinocandin; or any salts or variants thereof.
18. The method of paragraph 17, wherein the polyene antifungal agent is amphotericin B; candicidin; filipin; hamycin; natamycin; nystatin; or rimocidin.
19. The method of paragraph 17, wherein the imidazole antifungal agent is bifonazole; butoconazole; clotrimazole; econazole; fenticonzole; isoconazole; ketoconazole; miconazole; omoconazole; oxiconazole; sertaconazole; sulconazole; or tioconazole.
20. The method of paragraph 17, wherein the trizaole antifungal agent is albaconazole; fluconazole; isavuconazole; itraconazole; posaconazole; ravuconazole; terconazole; or voriconazole.
21. The method of paragraph 17, wherein the thiazole antifunal agent is abafungin.
22. The method of paragraph 17, wherein the allylamine antifungal agent is amorolfin; butenafine; naftifine; or terbinafine.
23. The method of paragraph 17, wherein the echinocandin is anidulafungin; caspofungin; or micafungin.
24. The method of any of paragraphs 1-16, wherein the antifungal agent is benzoic acid; ciclopirox; flucytosine; griseofulvin; haloprogin; polygodial; tolnaftate; undecylenic acid; or crystal violet.
25. The method of any of paragraphs 1-24, wherein the fungal infection is an infection of skin or soft tissue; a superficial mycosis; a cutaneous mycosis; a subcutaneous mycosis; a vaginal mycosis; a systemic mycosis; or is an infected wound or burn.
26. The method of any of paragraphs 1-25, wherein the infection is a surface wound, burn, or infection; infection of a mucosal surface; respiratory infection; infections of the eyes, ears, nose, or throat; or infection of an intestinal pathogen.
27. The method of any one of paragraph 1-26, wherein the fungal infection is resistant to one or more anti-fungal agents.
28. The method of any one of paragraphs 1-27, wherein the fungal infection involves one or more of:
   Candida spp.; Cryptococcus spp.; Aspergillus spp.; Microsporum spp.; Trichophyton spp.; Epidermophyton spp.; Trichosporon spp.; Fusarium spp.; Tinea versicolor; Tinea barbae; Tinea corporis; Tinea cruris; Tinea manuum; Tinea pedis; Tinea unguium; Tineafaciei; Tinea imbricate; Tinea incognito; Epidermophyton floccosum; Microsporum canis; Microsporum audouinii; Trichophyton interdigitale; Trichophyton mentagrophytes; Trichophyton tonsurans; Trichophyton schoenleini; Trichophyton rubrum; Hortaea werneckii; Piedraia hortae; Malasserzia furfur; Coccidioides immitis; Coccidioides posadasii; Histoplasma capsulatum; Histoplasma duboisii; Lacazia loboi; Paracoccidioides brasiliensis; Blastomyces dermatitidis; Sporothrix schenckii; Penicillium marneffei; Candida albicans; Candida glabrata; Candida tropicalis; Candida lusitaniae; Candidajirovecii; Candida krusei; Candida parapsilosi; Exophialajeanselmei; Fonsecaea pedrosoi; Fonsecasea compacta; Phialophora verrucosa; Geotrichum candidum; Pseudallescheria boydii; Rhizopus oryzae; Muco indicus; Absidia corymbifera; Synceplasastrum racemosum; Basidiobolus ranarum; Conidiobolus coronatus; Conidiobolus incongruous; Cryptococcus neoformans; Enterocytozoan bieneusi; Encephalitozoon intestinalis; and Rhinosporidium seeberi.
29. The method of any one of paragraphs 1-28, wherein the potentiator compound and the antifungal agent are co-formulated.
30. The method of paragraph 29, wherein the potentiator compound is glucose.
31. The method of any one of paragraphs 1 and 3-28, wherein the potentiator compound and the antifungal agent are administered separately.
32. The method of any one of paragraphs 1-31, wherein the potentiator compound is administered systemically or locally.
33. The method of any one of paragraphs 1-32, wherein the potentiator compound is administered intravenously, orally, or topically.
34. The method of any one of paragraphs 1-33, wherein the fungal infection occurs at or in a surface wound or burn, and the potentiator compound is administered topically to the affected area.
35. The method of any of paragraphs 1-33, wherein the potentiator compound is formulated as a cream, gel, foam, spray, or as a tablet or capsule for oral delivery.
36. A method for inhibiting fungal growth, the method comprising contacting a fungal cell with an effective amount of one or more potentiator compounds and an effective amount of an antifungal agent.
37. The method of paragraph 36, wherein the potentiator compound is an agonist of the RAS/PKA pathway; an agonist of the TCA cycle or respiration; an inhibitor of DNA repair; cAMP or a mimetic or analog thereof; a cAMP modulator, a phosphodiesterase inhibitor, or glucose.
38. The method of paragraph 37, wherein the agonist of the RAS/PKA pathway is an agonist of RAS1; RAS2; Cyr1; Cdc25; Srv2; Tpk1; Tpk2; Tpk3; and orthologs and homologs thereof; or an inhibitor Bcy1; Pde1; Pde2 or orthologs and homologs thereof.
39. The method of paragraph 38, wherein the inhibitor of Pde1 is IC224.
40. The method of paragraph 37, wherein the agonist of the TCA cycle or respiration is an agonist of Hap2; Hap3; Hap4; Hap5; Cit1; Cit2; Sdh1/2 or orthologs and homologs thereof.
41. The method of paragraph 37, wherein the potentiator compound modulates carbon source utilization or inhibits glucose utilization.
42. The method of paragraph 37, wherein the inhibitor of DNA repair is an inhibitor of double-strand break repair; an inhibitor of single-strand repair, or an inhibitor of direct reversal.
43. The method of paragraph 42, wherein the inhibitor of double-strand break repair is an inhibitor of Rad54; Rad51; Rad52; Rad5p; Rad57; RPA; Xrs2; Mre11; Lif1; Nej1; or orthologs and homologs thereof.
44. The method of paragraph 43, wherein the inhibitor is wortmannin; rapamycin; vorinostat; 0⁶-BG; NVP-BEZ235; 2-(Morpholin-4-yl)-benzo[h]chomen-4-one; 1-(2-hydroxy-4-morpholin-4-yl-phenyl)-ethanone; Ku55933; NU7441; or SU11752.
45. The method of paragraph 37, wherein the cAMP mimetic or analog or modulator thereof is diburtyryl cAMP; caffeine; forskolin; 8-bromo-cAMP; phorbol ester; sclareline; cholera toxin (CTx); aminophylline; 2,4 dinitrophenol (DNP); norepinephrine; epinephrine; isoproterenol; isobutylmethylxanthine (IBMX); theophylline (dimethylxanthine); dopamine; rolipram; iloprost; prostaglandin Et; prostaglandin E₂; pituitary adenylate cyclase activating polypeptide (PACAP); vasoactive intestinal polypeptide (VIP); (S)-adenosine; cyclic 3′,5′-(hydrogenphosphorothioate)triethyl ammonium; 8-bromoadenosine-3′,5′-cyclic monophosphate; 8-chloroadenosine-3′,5′-cyclic monophosphate; or N6,2′-O-dibutyryladenosine-3′,5′-cyclic monophosphate.
46. The method of paragraph 37, wherein the phosphodiesterase inhibitor is rolipram, mesembrine, drotaverine, roflumilast, ibudilast, piclamilast, luteolin, cilomilast, diazepam, arofylline, CP-80633, denbutylline, drotaverine, etazolate, filaminast, glaucine, HT-0712, ICI-63197, irsogladine, mesembrine, Ro20-1724, RPL-554, YM-976, sildenafil, vardenafil, tadalafil, udenafil, avanafil, sofyllin, pentoxifylline, acetildenafil, bucladesine, cilostamide, cilostazol, dipyridamole, enoximone, glaucine, ibudilast, icariin, inamrinone (formerly amrinone), lodenafil, luteolin, milrinone, mirodenafil, pimobendan, propentofylline, zardaverine, caffeine, theophylline, theobromine, 3-isobutyl-1-methylxanthine (IBMX), aminophylline, or paraxanthine.
47. The method of any one of paragraphs 36-46, wherein the potentiator is selected for its ability to increase ROS production or increase susceptibility to oxidative stress.
48. The method of paragraph 47, wherein the ROS is O₂⁻, H₂O₂, or O₂ and H₂O₂.
49. The method of any of paragraphs 36-48, wherein the antifungal is fungicidal or fungistatic.
50. The method of any of paragraphs 36-49, wherein the antifungal agent is a polyene; an imidazole; a triazole; a thiazole; an allylamine; and an echinocandin; or any salts or variants thereof.
51. The method of paragraph 50, wherein the polyene antifungal agent is amphotericin B; candicidin; filipin; hamycin; natamycin; nystatin; or rimocidin.
52. The method of paragraph 50, wherein the imidazole antifungal agent is bifonazole; butoconazole; clotrimazole; econazole; fenticonzole; isoconazole; ketoconazole; miconazole; omoconazole; oxiconazole; sertaconazole; sulconazole; or tioconazole.
53. The method of paragraph 50, wherein the trizaole antifungal agent is albaconazole; fluconazole; isavuconazole; itraconazole; posaconazole; ravuconazole; terconazole; or voriconazole.
54. The method of paragraph 50, wherein the thiazole antifunal agent is abafungin.
55. The method of paragraph 50, wherein the allylamine antifungal agent is amorolfin; butenafine; naftifine; or terbinafine.
56. The method of paragraph 50, wherein the echinocandin is anidulafungin; caspofungin; or micafungin.
57. The method of any of paragraphs 36-49, wherein the antifungal agent is benzoic acid; ciclopirox; flucytosine; griseofulvin; haloprogin; polygodial; tolnaftate; undecylenic acid; or crystal violet.
58. The method of any one of paragraphs 36-57, wherein the fungus is one or more of:
   Candida spp.; Cryptococcus spp.; Aspergillus spp.; Microsporum spp.; Trichophyton spp.; Epidermophyton spp.; Trichosporon spp.; Fusarium spp.; Tinea versicolor; Tinea barbae; Tinea corporis; Tinea cruris; Tinea manuum; Tinea pedis; Tinea unguium; Tineafaciei; Tinea imbricate; Tinea incognito; Epidermophyton floccosum; Microsporum canis; Microsporum audouinii; Trichophyton interdigitale; Trichophyton mentagrophytes; Trichophyton tonsurans; Trichophyton schoenleini; Trichophyton rubrum; Hortaea werneckii; Piedraia hortae; Malasserzia furfur; Coccidioides immitis; Coccidioides posadasii; Histoplasma capsulatum; Histoplasma duboisii; Lacazia loboi; Paracoccidioides brasiliensis; Blastomyces dermatitidis; Sporothrix schenckii; Penicillium marneffei; Candida albicans; Candida glabrata; Candida tropicalis; Candida lusitaniae; Candidajirovecii; Candida krusei; Candida parapsilosi; Exophialajeanselmei; Fonsecaea pedrosoi; Fonsecasea compacta; Phialophora verrucosa; Geotrichum candidum; Pseudallescheria boydii; Rhizopus oryzae; Muco indicus; Absidia corymbifera; Synceplasastrum racemosum; Basidiobolus ranarum; Conidiobolus coronatus; Conidiobolus incongruous; Cryptococcus neoformans; Enterocytozoan bieneusi; Encephalitozoon intestinalis; and Rhinosporidium seeberi.
59. The method of any one of paragraphs 36-58, wherein the potentiator compound and the antifungal agent are co-formulated.
60. The method of paragraph 59, wherein the potentiator compound is glucose.
61. A potentiator compound for use in inhibiting or treating a fungal infection, wherein the potentiator compound is an agonist of the RAS/PKA pathway; an agonist of the TCA cycle or respiration; an inhibitor of DNA repair; cAMP or a mimetic or analog thereof; a cAMP modulator, a phosphodiesterase inhibitor, or glucose.
62. The compound of paragraph 61, wherein the agonist of the RAS/PKA pathway is an agonist of RAS1; RAS2; Cyr1; Cdc25; Srv2; Tpk1; Tpk2; Tpk3; or orthologs and homologs thereof; or an inhibitor of Bcy1; PdeI; or orthologs and homologs thereof.
63. The compound of paragraph 62, wherein the inhibitor of PdeI is IC224.
64. The compound of paragraph 61, wherein the agonist of the TCA cycle or respiration is an agonist of Hap2; Hap3; Hap4; Hap5; Cit1; Cit2; Sdh1/2 or orthologs and homologs thereof.
65. The compound of paragraph 61, wherein the potentiator compound modulates carbon source utilization or inhibits glucose utilization.
66. The compound of paragraph 61, wherein the inhibitor of DNA repair is an inhibitor of double-strand break repair; an inhibitor of single-strand repair, or an inhibitor of direct reversal.
67. The compound of paragraph 66, wherein the inhibitor of double-strand break repair is an inhibitor of Rad54; Rad51; Rad52; Rad55; Rad57; RPA; Xrs2; Mre11; Lif1; Nej1; or orthologs and homologs thereof.
68. The compound of paragraph 67, wherein the inhibitor is wortmannin; rapamycin; vorinostat; 0⁶-BG; NVP-BEZ235; 2-(Morpholin-4-yl)-benzo[h]chomen-4-one; 1-(2-hydroxy-4-morpholin-4-yl-phenyl)-ethanone; Ku55933; NU7441; or SU11752.
69. The compound of paragraph 61, wherein the cAMP mimetic or analog or modulator thereof is diburtyryl cAMP; caffeine; forskolin; 8-bromo-cAMP; phorbol ester; sclareline; cholera toxin (CTx); aminophylline; 2,4 dinitrophenol (DNP); norepinephrine; epinephrine; isoproterenol; isobutylmethylxanthine (IBMX); theophylline (dimethylxanthine); dopamine; rolipram; iloprost; prostaglandin Et; prostaglandin E₂; pituitary adenylate cyclase activating polypeptide (PACAP); vasoactive intestinal polypeptide (VIP); (S)-adenosine; cyclic 3′,5′-(hydrogenphosphorothioate)triethyl ammonium; 8-bromoadenosine-3′,5′-cyclic monophosphate; 8-chloroadenosine-3′,5′-cyclic monophosphate; or N6,2′-O-dibutyryladenosine-3′,5′-cyclic monophosphate.
70. The composition of paragraph 61, wherein the phosphodiesterase inhibitor is rolipram, mesembrine, drotaverine, roflumilast, ibudilast, piclamilast, luteolin, cilomilast, diazepam, arofylline, CP-80633, denbutylline, drotaverine, etazolate, filaminast, glaucine, HT-0712, ICI-63197, irsogladine, mesembrine, Ro20-1724, RPL-554, YM-976, sildenafil, vardenafil, tadalafil, udenafil, avanafil sofyllin, pentoxifylline, acetildenafil, bucladesine, cilostamide, cilostazol, dipyridamole, enoximone, glaucine, ibudilast, icariin, inamrinone (formerly amrinone), lodenafil, luteolin, milrinone, mirodenafil, pimobendan, propentofylline, zardaverine, caffeine, theophylline, theobromine, 3-isobutyl-1-methylxanthine (IBMX), aminophylline, or paraxanthine.
71. The compound of any of paragraphs 61-70, wherein the potentiator is selected for its ability to increase ROS production or increase susceptibility to oxidative stress.
72. The compound of paragraph 71, wherein the ROS is O₂⁻, H₂O₂, or O₂⁻ and H₂O₂.
73. The compound of any of paragraphs 61-72, wherein the potentiator compound is coformulated with an antifungal.
74. The compound of paragraph 73, wherein the antifungal is fungicidal or fungistatic.
75. The compound of any of paragraphs 61-74, wherein the antifungal agent is a polyene; an imidazole; a triazole; a thiazole; an allylamine; and an echinocandin; or any salts or variants thereof.
76. The compound of paragraph 75, wherein the polyene antifungal agent is amphotericin B; candicidin; filipin; hamycin; natamycin; nystatin; or rimocidin.
77. The compound of paragraph 75, wherein the imidazole antifungal agent is bifonazole; butoconazole; clotrimazole; econazole; fenticonzole; isoconazole; ketoconazole; miconazole; omoconazole; oxiconazole; sertaconazole; sulconazole; or tioconazole.
78. The compound of paragraph 75, wherein the trizaole antifungal agent is albaconazole; fluconazole; isavuconazole; itraconazole; posaconazole; ravuconazole; terconazole; or voriconazole.
79. The compound of paragraph 75, wherein the thiazole antifunal agent is abafungin.
80. The compound of paragraph 75, wherein the allylamine antifungal agent is amorolfin; butenafine; naftifine; or terbinafine.
81. The compound of paragraph 75, wherein the echinocandin is anidulafungin; caspofungin; or micafungin.
82. The compound of any of paragraphs 61-74, wherein the antifungal agent is benzoic acid; ciclopirox; flucytosine; griseofulvin; haloprogin; polygodial; tolnaftate; undecylenic acid; or crystal violet.
83. The compound of any of paragraphs 61-85, wherein the potentiator compound is glucose.
84. A composition comprising an antifungal agent formulated in a glucose solution.
85. The composition of paragraph 84, wherein the antifungal is fungicidal or fungistatic.
86. The composition of any of paragraphs 84-85, wherein the antifungal agent is a polyene; an imidazole; a triazole; a thiazole; an allylamine; and an echinocandin; or any salts or variants thereof.
87. The composition of paragraph 86, wherein the polyene antifungal agent is amphotericin B; candicidin; filipin; hamycin; natamycin; nystatin; rimocidin;
88. The composition of paragraph 86, wherein the imidazole antifungal agent is bifonazole; butoconazole; clotrimazole; econazole; fenticonzole; isoconazole; ketoconazole; miconazole; omoconazole; oxiconazole; sertaconazole; sulconazole; or tioconazole.
89. The composition of paragraph 86, wherein the trizaole antifungal agent is albaconazole; fluconazole; isavuconazole; itraconazole; posaconazole; ravuconazole; terconazole; or voriconazole.
90. The compositon of paragraph 86, wherein the thiazole antifunal agent is abafungin.
91. The composition of paragraph 86, wherein the allylamine antifungal agent is amorolfin; butenafine; naftifine; or terbinafine.
92. The compositon of paragraph 86, wherein the echinocandin is anidulafungin; caspofungin; or micafungin.
93. The composition of any of paragraphs 84-85, wherein the antifungal agent is benzoic acid; ciclopirox; flucytosine; griseofulvin; haloprogin; polygodial; tolnaftate; undecylenic acid; or crystal violet.
94. A method comprising selecting a compound that increases ROS production in a target fungal pathogen or that increases ROS-induced cellular damage in the target fungal pathogen, and formulating the compound for treatment of a fungal pathogen, optionally with one or more additional antifungal agents.
95. The method of paragraph 94, wherein the compound increases ROS production by modulating fungal respiration.
96. The method of paragraph 95, wherein the compound increases TCA cycle or electron transport chain activity.
97. The method of paragraph 95, wherein the compound that increased TCA cycle or electron transport chain activity is an agonist of Hap2; Hap3; Hap4; Hap5; Cit1; Cit2; Sdh1/2 or orthologs and homologs thereof.
98. The method of paragraph 95, wherein the compound activates the RAS/PKA pathway.
99. The method of paragraph 98, wherein the compound that activates the RAS/PKA pathway is an agonist of RAS1; RAS2; Cyr1; Cdc25; Srv2; Tpk1; Tpk2; Tpk3; and orthologs and homologs thereof; or an inhibitor of Bcy1; Pde1; Pde2 or orthologs and homologs thereof.
100. The method of paragraph 99, wherein the inhibitor of Pde1 is IC224.
101. The method of any of paragraphs 94-100, wherein the compound increases ROS-induced cellular damage.
102. The method of any of paragraphs 94-95 or 101, wherein the compound inhibits DNA damage repair.
103. The method of paragraph 102, wherein the inhibitor of DNA repair is an inhibitor of double-strand break repair; an inhibitor of single-strand repair, or an inhibitor of direct reversal.
104. The method of paragraph 103, wherein the inhibitor of double-strand break repair is an inhibitor of Rad54; Rad51; Rad52; Rad55; Rad57; RPA; Xrs2; Mre11; Lif1; Nej1; or orthologs and homologs thereof.
105. The method of paragraph 104, wherein the inhibitor is wortmannin; rapamycin; vorinostat; 0⁶-BG; NVP-BEZ235; 2-(Morpholin-4-yl)-benzo[h]chomen-4-one; 1-(2-hydroxy-4-morpholin-4-yl-phenyl)-ethanone; Ku55933; NU7441; or SU11752.
106. The method of any of paragraphs 94-95 or 101, wherein the compound is cAMP, a cAMP mimetic or analog or modulator thereof.
107. The method of paragraph 106, wherein the cAMP mimetic or analog or modulator thereof is diburtyryl cAMP; caffeine; forskolin; 8-bromo-cAMP; phorbol ester; sclareline; cholera toxin (CTx); aminophylline; 2,4 dinitrophenol (DNP); norepinephrine; epinephrine; isoproterenol; isobutylmethylxanthine (IBMX); theophylline (dimethylxanthine); dopamine; rolipram; iloprost; prostaglandin E₁; prostaglandin E₂; pituitary adenylate cyclase activating polypeptide (PACAP); vasoactive intestinal polypeptide (VIP); (S)-adenosine; cyclic 3′,5′-(hydrogenphosphorothioate)triethyl ammonium; 8-bromoadenosine-3′,5′-cyclic monophosphate; 8-chloroadenosine-3′,5′-cyclic monophosphate; or N6,2′-O-dibutyryladenosine-3′,5′-cyclic monophosphate.
108. The method of any of paragraphs 94-95 or 101, wherein the compound is a phosphodiesterase inhibitor.
109. The method of paragraph 108, wherein the phosphodiesterase inhibitor is rolipram, mesembrine, drotaverine, roflumilast, ibudilast, piclamilast, luteolin, cilomilast, diazepam, arofylline, CP-80633, denbutylline, drotaverine, etazolate, filaminast, glaucine, HT-0712, ICI-63197, irsogladine, mesembrine, Ro20-1724, RPL-554, YM-976, sildenafil, vardenafil, tadalafil, udenafil, avanafil, sofyllin, pentoxifylline, acetildenafil, bucladesine, cilostamide, cilostazol, dipyridamole, enoximone, glaucine, ibudilast, icariin, inamrinone (formerly amrinone), lodenafil, luteolin, milrinone, mirodenafil, pimobendan, propentofylline, zardaverine, caffeine, theophylline, theobromine, 3-isobutyl-1-methylxanthine (IBMX), aminophylline, or paraxanthine.
110. The method of any of paragraphs 94-109, wherein the antifungal is fungicidal or fungistatic.
111. The method of any of paragraphs 94-110, wherein the antifungal agent is a polyene; an imidazole; a triazole; a thiazole; an allylamine; or an echinocandin; or any salts or variants thereof.
112. The method of paragraph 110, wherein the polyene antifungal agent is amphotericin B; candicidin; filipin; hamycin; natamycin; nystatin; or rimocidin.
113. The method of paragraph 110, wherein the imidazole antifungal agent is bifonazole; butoconazole; clotrimazole; econazole; fenticonzole; isoconazole; ketoconazole; miconazole; omoconazole; oxiconazole; sertaconazole; sulconazole; or tioconazole.
114. The method of paragraph 110, wherein the trizaole antifungal agent is albaconazole; fluconazole; isavuconazole; itraconazole; posaconazole; ravuconazole; terconazole; or voriconazole.
115. The method of paragraph 110, wherein the thiazole antifunal agent is abafungin.
116. The method of paragraph 110, wherein the allylamine antifungal agent is amorolfin; butenafine; naftifine; or terbinafine.
117. The method of paragraph 110, wherein the echinocandin is anidulafungin; caspofungin; or micafungin.
118. The method of any of paragraphs 94-110, wherein the antifungal agent is benzoic acid; ciclopirox; flucytosine; griseofulvin; haloprogin; polygodial; tolnaftate; undecylenic acid; or crystal violet.
119. The method of any one of paragraphs 94-118, wherein the fungal pathogen is resistant to one or more anti-fungal agents.
120. The method of any one of paragraphs 94-119, wherein the fungal pathogen is Candida spp.; Candida spp.; Cryptococcus spp.; Aspergillus spp.; Microsporum spp.; Trichophyton spp.; Epidermophyton spp.; Trichosporon spp.; Fusarium spp.; Tinea versicolor; Tinea barbae; Tinea corporis; Tinea cruris; Tinea manuum; Tinea pedis; Tinea unguium; Tineafaciei; Tinea imbricate; Tinea incognito; Epidermophytonfloccosum; Microsporum canis; Microsporum audouinii; Trichophyton interdigitale; Trichophyton mentagrophytes; Trichophyton tonsurans; Trichophyton schoenleini; Trichophyton rubrum; Hortaea werneckii; Piedraia hortae; Malasserzia furfur; Coccidioides immitis; Coccidioides posadasii; Histoplasma capsulatum; Histoplasma duboisii; Lacazia loboi; Paracoccidioides brasiliensis; Blastomyces dermatitidis; Sporothrix schenckii; Penicillium marneffei; Candida albicans; Candida glabrata; Candida tropicalis; Candida lusitaniae; Candida jirovecii; Candida krusei; Candida parapsilosi; Exophialajeanselmei; Fonsecaea pedrosoi; Fonsecasea compacta; Phialophora verrucosa; Geotrichum candidum; Pseudallescheria boydii; Rhizopus oryzae; Muco indicus; Absidia corymbifera; Synceplasastrum racemosum; Basidiobolus ranarum; Conidiobolus coronatus; Conidiobolus incongruous; Cryptococcus neoformans; Enterocytozoan bieneusi; Encephalitozoon intestinalis; and Rhinosporidium seeberi.
121. The method of any of paragraphs 94-120, wherein the compound is formulated as a cream, gel, foam, spray, or as a tablet or capsule for oral delivery.

### EXAMPLES

### Example 1

Described herein are results demonstrating that 1) fungicide-dependent ROS production leads to fungal cellular death; 2) the TCA, ETC and RAS/PKA pathways are involved in fungicide-induced cellular death; 3) antifungal agents elevate mitochondrial activity, the AMP/ATP ratio, and sugar production; and 4) DNA damage plays an important role in fungicide-induced cellular death.

Amphotericin, miconazole and ciclopirox are antifungal agents from three different drug classes that can effectively kill planktonic yeast, yet their complete fungicidal mechanisms are not fully understood. Employed herein is a systems biology approach to identify a common oxidative damage cellular death pathway triggered by these representative fungicides in Candida albicans and Saccharomyces cerevisiae. This mechanism utilizes a signaling cascade involving the GTPases Ras1/2 and Protein Kinase A, and culminates in death through the production of toxic ROS in a tricarboxylic acid cycle- and respiratory chain-dependent manner. It is also demonstrated herein that the metabolome of C. albicans is altered by antifungal drug treatment, exhibiting a shift from fermentation to respiration, a jump in the AMP/ATP ratio, and elevated production of sugars; this coincides with elevated mitochondrial activity. Lastly, it is demonstrated herein that DNA damage plays a critical role in antifungal-induced cellular death and that blocking DNA repair mechanisms potentiates fungicidal activity.

A rapid rise in immunocompromised patients over the past five decades has led to increasing incidence of systemic fungal infections. Despite current treatment options, the morbidity and mortality rates associated with fungal infections, particularly those of Candida species, remain high (Ostrosky-Zeichner et al., 2010).

The polyene amphotericin B (AMB), introduced in the late 1950s, was the first widely used antifungal (AF) drug (Ostrosky-Zeichner et al., 2010). Due to its strong hydrophobicity, AMB penetrates the fungal membrane and binds to ergosterol leading to membrane damage. Azoles, a second class of AFs, became available in the 1980s and act by inhibiting ergosterol biosynthesis to induce the accumulation of a toxic methylated sterol that stops cell growth (Ostrosky-Zeichner et al., 2010). While azoles tend to be fungistatic due to their poor solubility, under certain conditions and formulations, some azoles such as miconazole (MCZ) can be fungicidal (Thevissen et al., 2007). Unlike AMB and MCZ, the primary targets of the synthetic AF cicloplrox olamine (CIC) are not fully understood, though some evidence indicates that CIC acts by affecting DNA repair or directly inducing DNA damage (Leem et al., 2003).

As described herein, a systems biology approach was used to identify mechanisms by which the aforementioned AFs—AMB, MCZ and CIC—lead to fungal cellular death. Despite their different primary modes of action, all three classes of fungicidal drugs induce a common oxidative damage cellular death pathway in S. cerevisiae and C. albicans that involves alterations to cellular metabolism and respiration, culminating in the formation of lethal ROS.

### Results

Fungicide-Dependent ROS Production Leads to Fungal Cell Death.

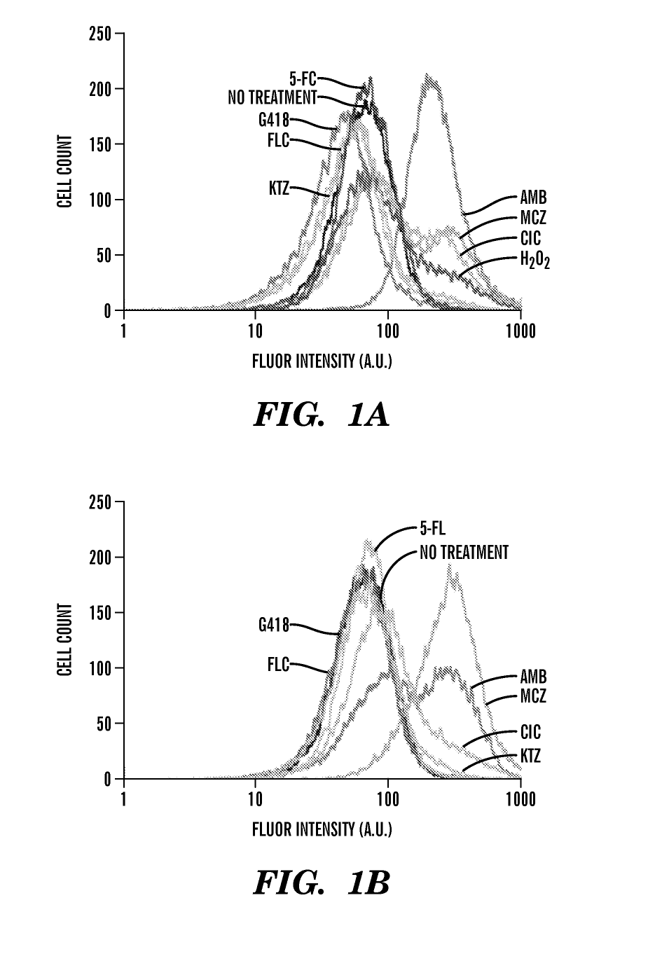

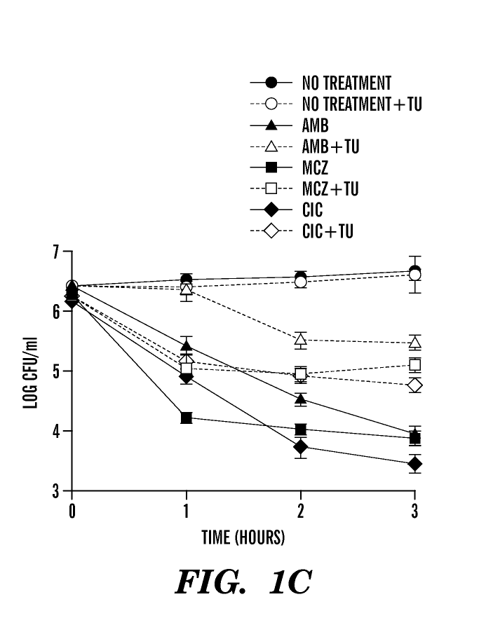

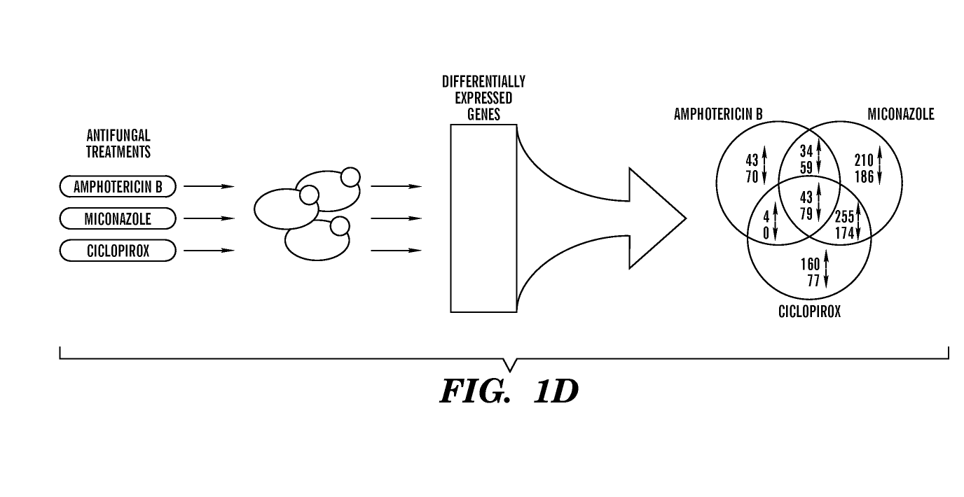

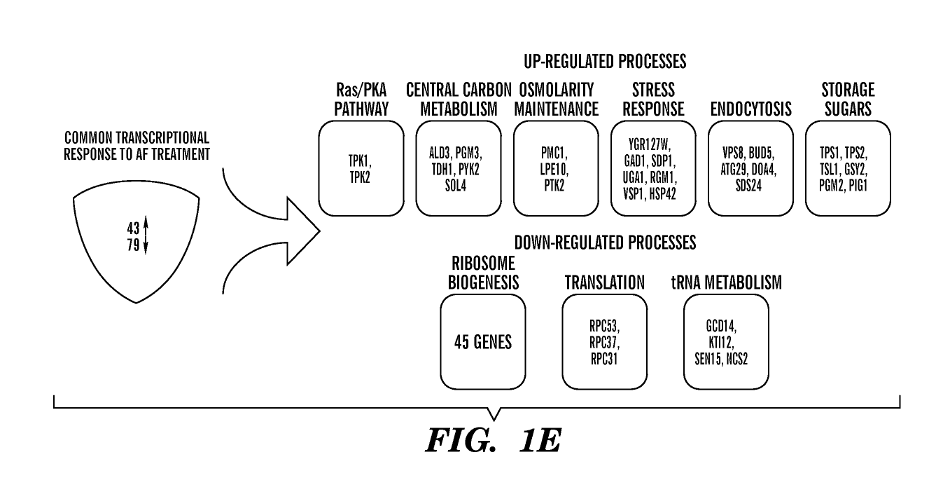

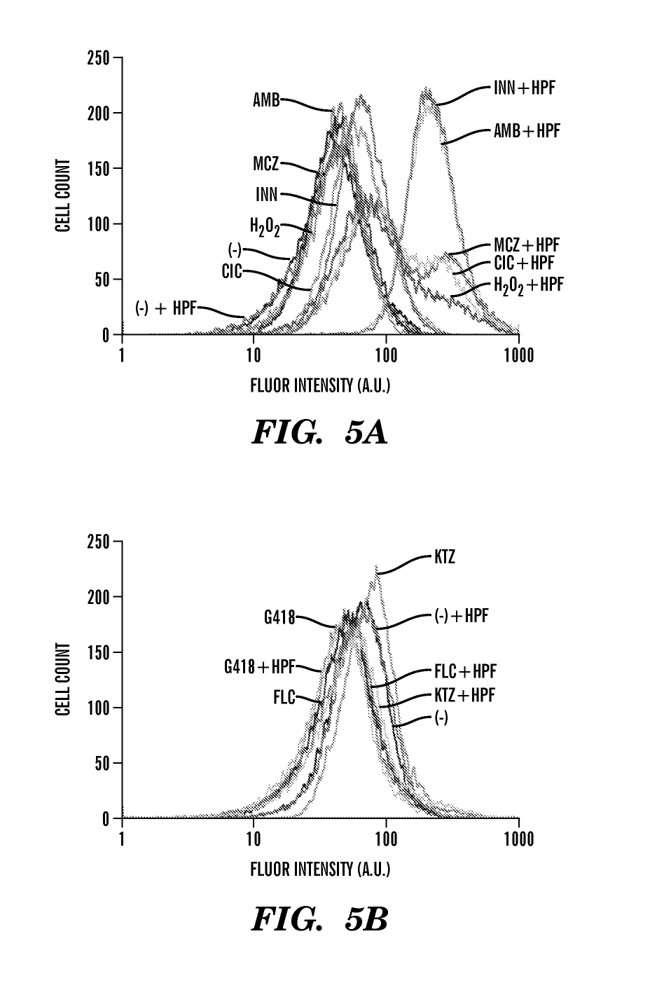

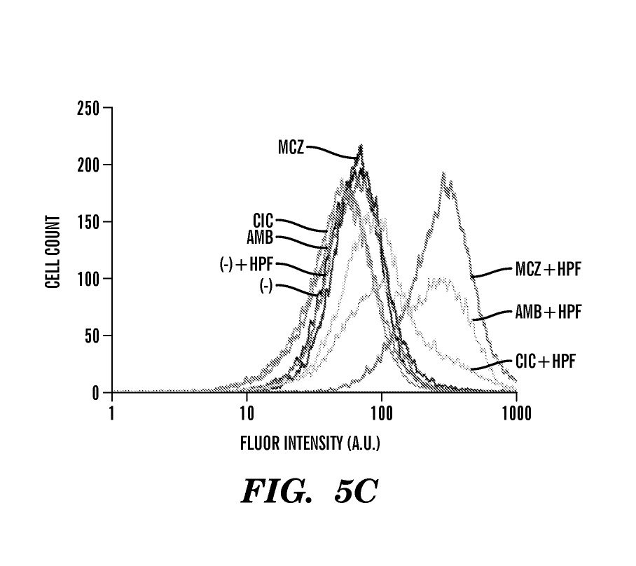

The formation of ROS following AF treatment in yeast was measured using the dye 3′-(p-hydroxyphenyl) fluorescein (HPF), which is preferentially oxidized by intracellular hydroxyl radicals into a fluorescent product (Kohanski et al., 2007). Exponentially growing wildtype S. cerevisiae and C. albicans were treated in synthetic dextrose complete (SDC) medium with the minimum concentration of fungicide required to achieve at least a 90% reduction in colony forming units (CFU) after three hours of exposure. As a positive control, cells were also treated with H₂O₂, a potent inducer of hydroxyl radical formation (Perrone et al., 2008). After 1.5 hours of treatment, all tested fungicidal drugs and H₂O₂ lead to dramatic induction of HPF fluorescence (FIGS. 1A, 1B and 5A-5C), indicating that all tested fungicidal agents induce the formation of ROS. Conversely, fungistatic drugs added at concentrations 10-fold above the minimal inhibitory concentration or at the maximum soluble concentration did not lead to detectable ROS formation (FIGS. 1A, 1B and 5A-5C).

To test whether the observed production of ROS contributes to AF-induced cellular death, cells were treated with thiourea, a potent scavenger of hydroxyl radicals in eukaryotic and prokaryotic cells (Kohanski et al., 2007; Touati et al., 1995). Exposing exponentially growing S. cerevisiae to 50 mM thiourea for 30 minutes prior to the addition of AF drugs considerably diminished the toxicity of all three AFs, reducing killing at 3 hours by ˜15-fold for AMB and CIC and by ˜10-fold for MCZ (FIG. 1C). These results indicate that ROS production plays a critical role in cellular death following treatment by AMB, MCZ and CIC.

Identifying a Common Transcriptional Response to AF Treatment.

To build a comprehensive model of the yeast response to AFs, changes in global gene expression of S. cerevisiae following treatment with AMB, MCZ and CIC were measured. To assess the effect of each AF treatment on gene expression, a z-test was performed between experiment and control samples, where the average and standard deviation for the expression of each gene was calculated across a compendium of publically available expression datasets. The differential gene expression profile of each treatment at each time point was compared to the no-treatment control to identify the set of genes commonly perturbed by fungicide treatment (FIG. 1D). This analysis revealed that 43 genes were commonly upregulated under AF treatment, while 79 genes were downregulated under the same conditions (FIG. 1E). To investigate the biological pathways and processes in which these genes are involved, a functional enrichment analysis on the GO terms associated with each one of these genes was run. The analysis was conducted with the Saccharomyces Genome Database's GO Term Finder tool using default parameters. The majority of the commonly downregulated genes are involved in protein synthesis, specifically, ribosomal biogenesis and tRNA synthesis. The 43 commonly upregulated genes fall into six general biological processes: the production of storage sugars, endocytosis, general stress response, osmolarity maintenance, central carbon metabolism, and the RAS/PKA signaling pathway (FIG. 1E).

This expression analysis identified the production of storage sugars as a key process upregulated following AF treatment. Specifically, genes involved in glycogen metabolism and the production of the storage sugar trehalose were robustly upregulated after the addition of fungicides (FIG. 1E; Table 2). Cellular production of trehalose and glycogen are energy expensive pathways that consume significant amounts of ATP. Consistent with this, the activation of the trehalose pathway has been shown to increase ATP consumption, mitochondrial enzyme content and respiration (Noubhani et al., 2009). Thus, the production of these sugars can contribute to increased mitochondrial activity and elevated ROS production.

TCA Cycle and Electron Transport Chain Play Critical Roles in AF-Induced Cell Death.

To identify genes critical to AF action, the AF sensitivity of single-gene knockouts identified through the common transcriptional analysis was tested. In total, the AF sensitivities of 81 single-gene knockouts (Table 1) was tested, and 12 of them were found to have increased resistance to all three antifungals (AMB, MCZ, CIC).

Actively respiring, energy-producing mitochondria are the major source of ROS in yeast cells. However, S. cerevisiae respiratory activity is usually repressed under normal growth conditions in the presence of glucose (Hardie et al., 1999), though various stress responses have been shown to upregulate respiratory genes even in the presence of glucose (Gasch et al., 2000). It was therefore hypothesized that treatment with fungicides induces a switch from normal fermentative growth to mitochondrial respiration.

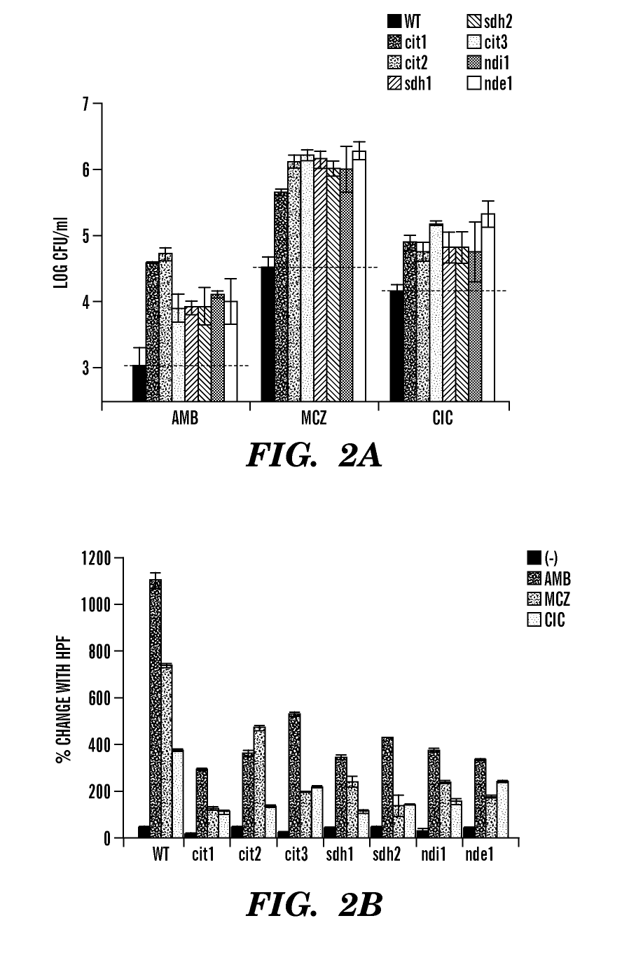

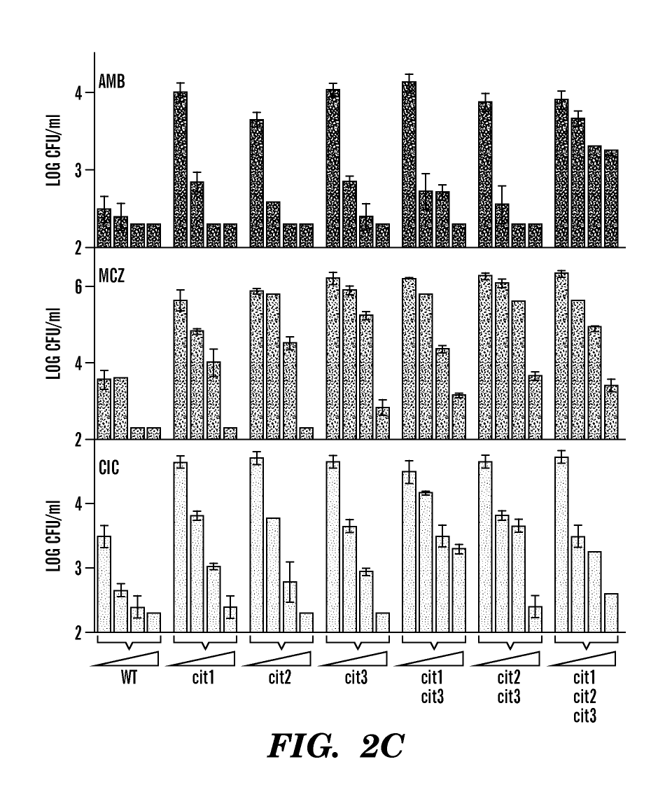

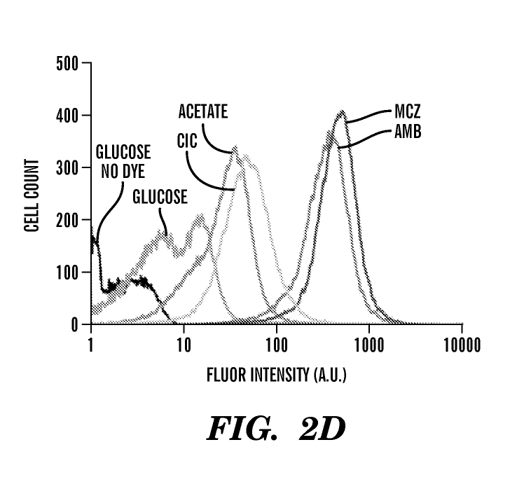

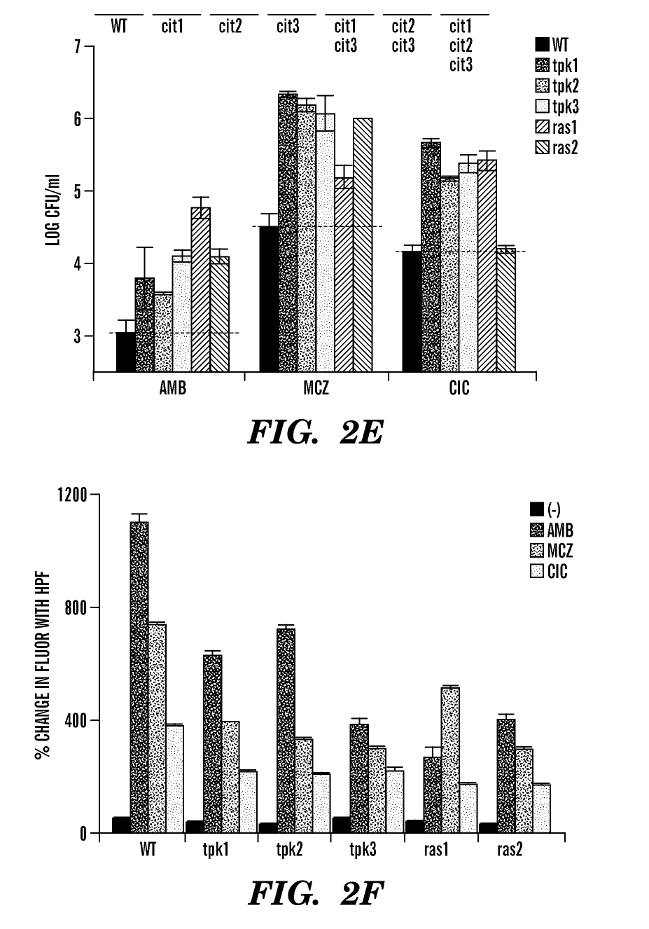

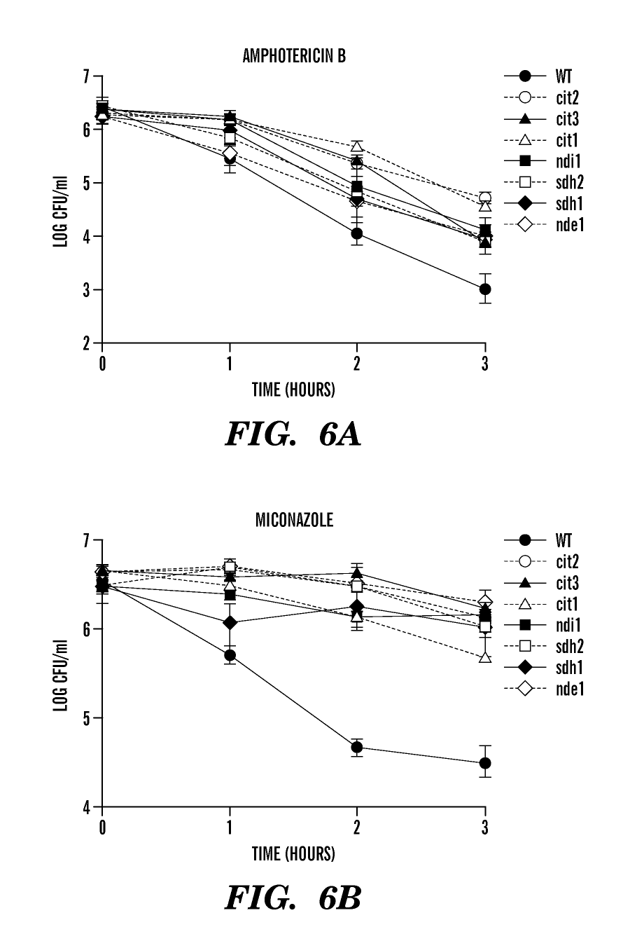

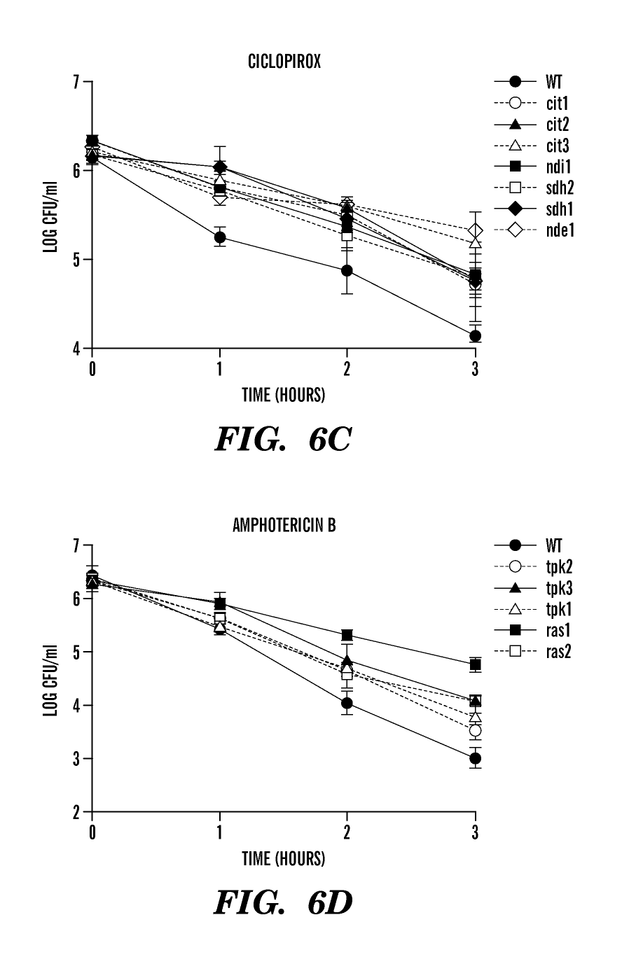

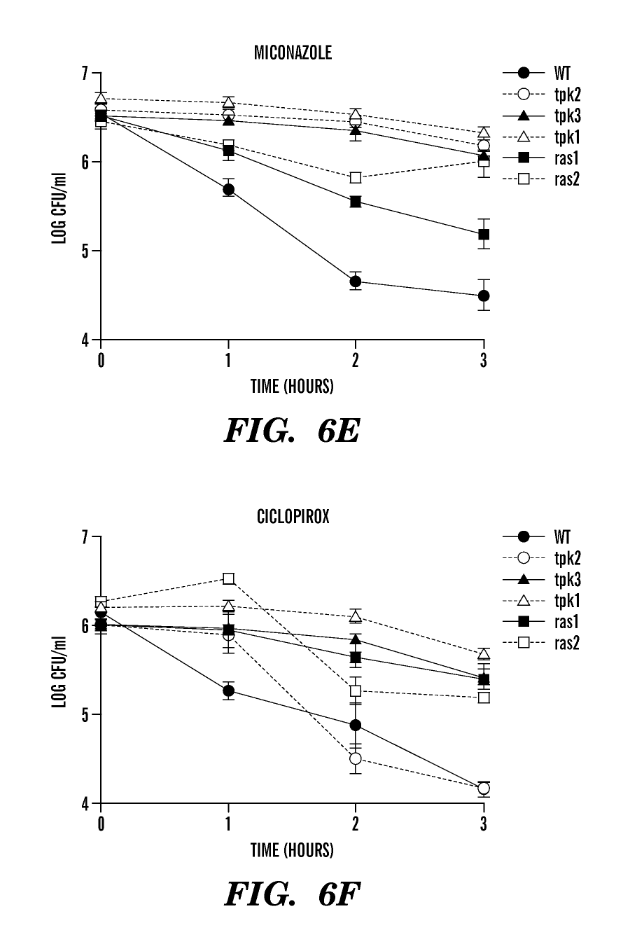

To assess the role of mitochondrial respiration in AF-induced cellular death, the expression of mitochondrial genes in S. cerevisiae in response to AF treatment was analyzed (Table 2). Of particular interest, the gene encoding the rate-limiting step of the TCA cycle, citrate synthase-1 (CIT1), exhibited a slight increase in expression across all AF treatments. The induction of CIT1 is a hallmark of stress-induced respiration (Gasch et al., 2000). Deleting CIT1, CIT2 or CIT3 dramatically reduced yeast sensitivity to all three AFs, compared to the wildtype S. cerevisiae strain (FIGS. 2A and 6A-6F). To parse out the role of the various citrate syntheses in AF-induced cellular death, single and double deletions of CIT1 and CIT2 were created in the cit3 background. Deleting the remaining citrate synthases in this background provides additive resistance to AMB, with the triple mutant requiring more than 8-fold higher drug concentrations to achieve the sensitivity of the single mutants (FIG. 2C). Interestingly, a similar additive effect was not observed with MCZ or CIC, indicating that AMB toxicity is more sensitive to further changes in citrate metabolism than the other two drugs.

Deleting succinate dehydrogenases (SDH1 or SDH2), enzymes that couple the oxidation of succinate to the transfer of electrons to the mitochondrial ETC (Chapman et al., 1992), decreased drug susceptibility. Additionally, blocking the TCA cycle at these two key points reduces the AF-dependent production of ROS (FIG. 2B), indicating that this phenomenon involves the TCA cycle.

The TCA cycle produces NADH, which is then fed into the ETC to produce ATP. This activity also leads to the production of ROS as a byproduct of aerobic respiration. The first committed steps of the ETC were targeted by deleting the intramitochondrial NADH dehydrogenase (NDI1) and the external NADH dehydrogenase (NDE1). The NDI1 and NDE1 deletions exhibited increased resistance to all three AF drugs and a concomitant reduction in AF-dependent ROS production (FIGS. 2A and 2B).

It was next sought to assess the mitochondrial activity of AF-treated fungal cells. Yhe MitoTracker Red dye, which enters the mitochondrial matrix by utilizing the proton motive force and thus labels metabolically active mitochondria in viable cells was used (Arita et al., 2006; Tomas et al., 2011). Exponential phase S. cerevisiae were incubated in glucose-free synthetic complete (SC) media for 30 min and then switched to SC containing 2% glucose or non-fermentable acetate for 1.5 hours. As expected, approximately 5-fold more MitoTracker fluorescence was detected in acetate-incubated cells (FIG. 2D), indicating that the dye specifically labels cells with activated mitochondria. Adding CIC, AMB, or MCZ to glucose-incubated cells increased MitoTracker fluorescence by 10-fold, 60-fold and 70-fold, respectively (FIG. 2D). All three drugs induced considerably more mitochondrial activity than acetate, indicating that AF treatment has a greater impact on mitochondrial activity than normal respiratory metabolism. These results are consistent with our hypothesis that AF drugs induce a shift from fermentative growth to ROS-producing mitochondrial respiration.

RAS/PKA Pathway is a Key Mediator of Antifungal Toxicity.

Having established that AFs induce mitochondrial-dependent ROS production, it was next sought to ascertain the signaling events that lead to these metabolic changes. The RAS/PKA pathway, consisting of two GTPases Ras1 and 2 and three Protein Kinase A isoforms (Tpk1-3), has been shown to respond to cellular stress by inducing mitochondrial biogenesis and cellular death through the production of ROS via disordered mitochondrial respiration (Chevtzoff et al., 2010; Leadsham and Gourlay, 2010; Thevelein and de Winde, 1999). Aspects of this signaling pathway were upregulated in response to AFs (Table 2). For example, TPK1 and TPK2 were robustly induced in response to AF treatment, and an increase in expression of other genes involved in the same pathway was found (Table 2), namely BMH2, CYR1 and SR V2. Further, PDE2, the cAMP phosphodiesterase that represses the RAS/PKA signaling, was significantly downregulated. Deleting PDE2 in the presence of activated RAS/PKA signaling has been shown to lead to the overproduction of ROS by dysfunctional mitochondria resulting in cellular death (Leadsham and Gourlay, 2010). Together, these results indicate that the RAS/PKA signaling pathway is largely upregulated in response to AF treatment and may contribute to the production of ROS by mitochondria.

The role of the RAS/PKA pathway in AF-induced cellular death was tested by studying single-gene knockouts of the upstream and downstream portions of the pathway. Deleting either RAS1 or RAS2 reduced yeast susceptibility to all three AFs (FIGS. 2E and 6A-6F), and deleting the downstream effector kinases, TPK1, TPK2 and TPK3, also reduced or delayed killing. Additionally, deleting any one of the key members of the RAS/PKA signaling pathway reduced the drug-dependent buildup of ROS (FIG. 2F). This finding suggests that RAS/PKA signaling is critical for the induction of mitochondrial ROS production in response to drug treatment.

Common Metabolic Changes Resulting from Antifungal Treatment.

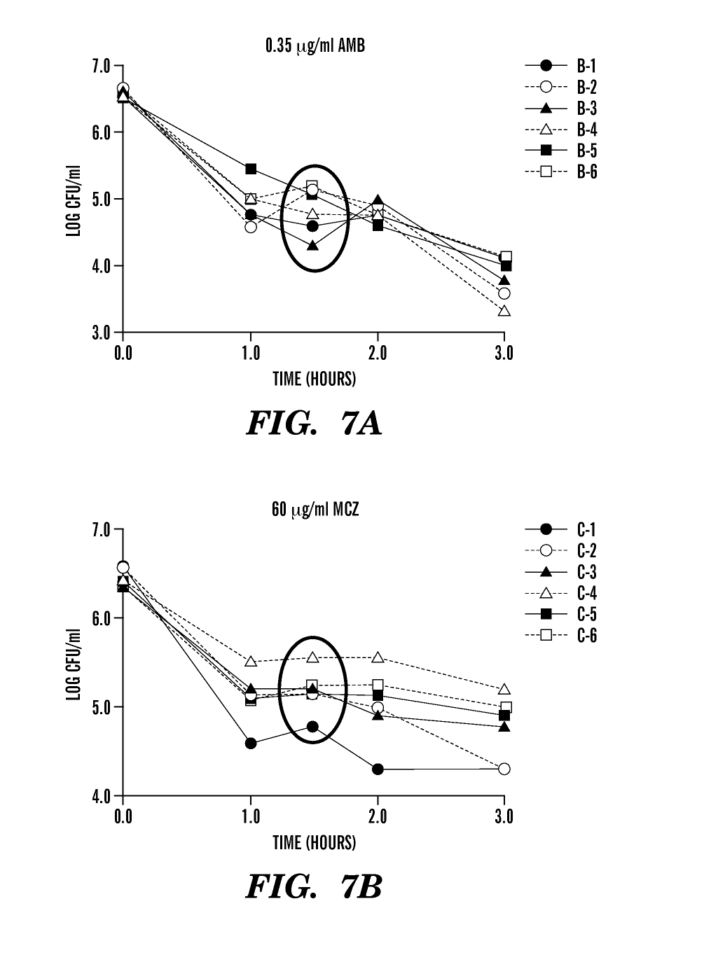

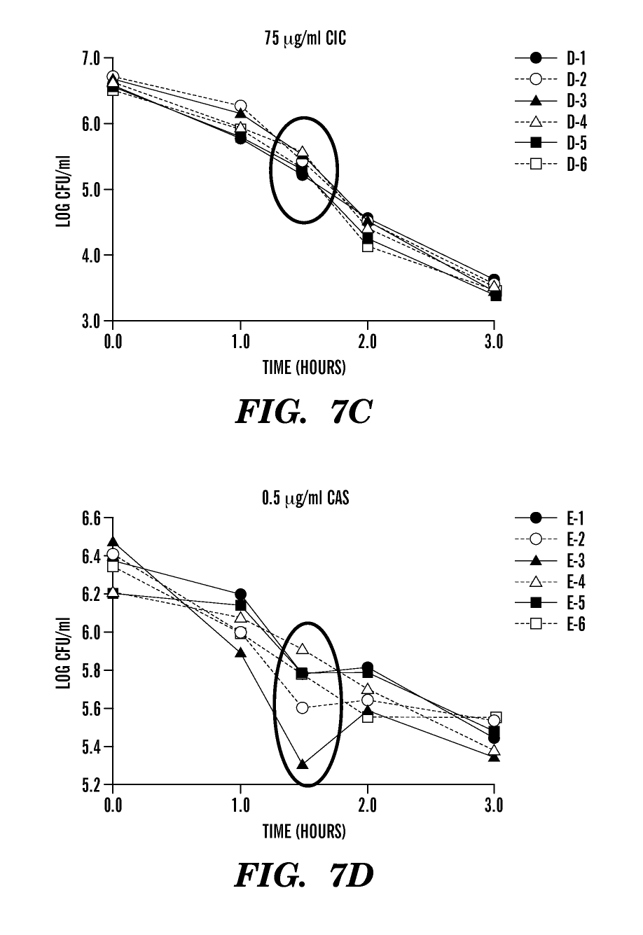

The results described herein link AF treatment to distinct changes in intracellular metabolic activity as part of an induced common killing mechanism. To further explore the role of metabolism in AF-induced cellular death, the effect of AF treatment on the intracellular metabolome of C. albicans was measured. To do so, a platform for metabolite detection and relative quantification from Metabolon Inc. (Durham, USA) (Evans et al., 2009) was utilized. Exponentially growing C. albicans cells were treated with AFs and samples collected for metabolomic analysis after 1.5 hours of treatment, a time point when it was expected that at least 90% of cells contributing to the metabolite pool would be irreversibly committed to the death pathway (FIGS. 7A-7D). The three drug treatments (AMB, MCZ, CIC) significantly changed (p≦0.05) the relative abundance of between 155 and 213 metabolites, compared to the no-treatment controls.

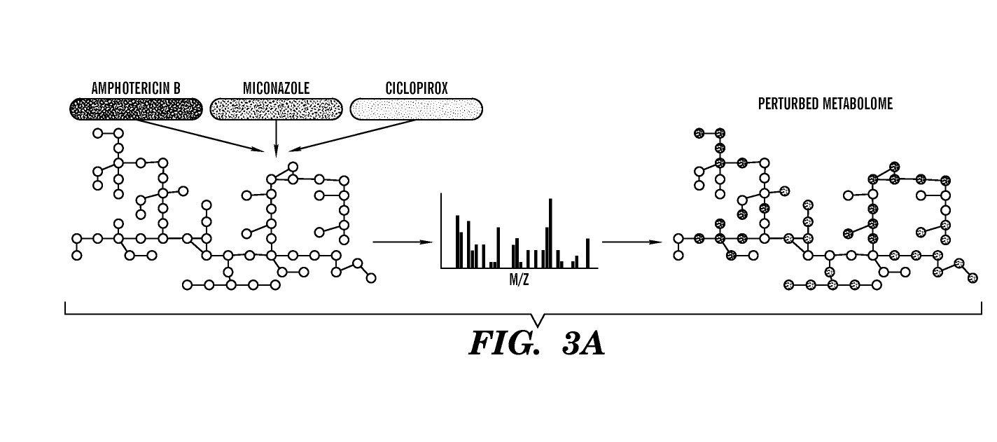

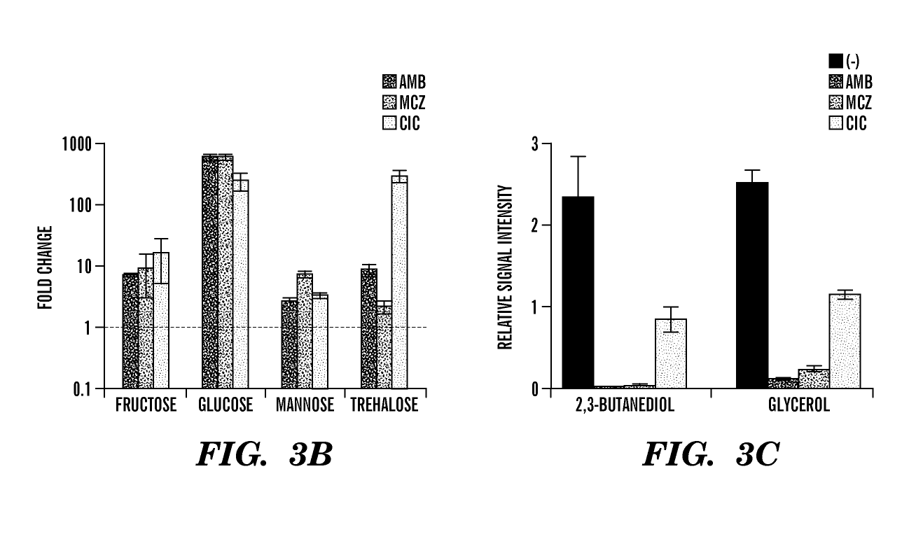

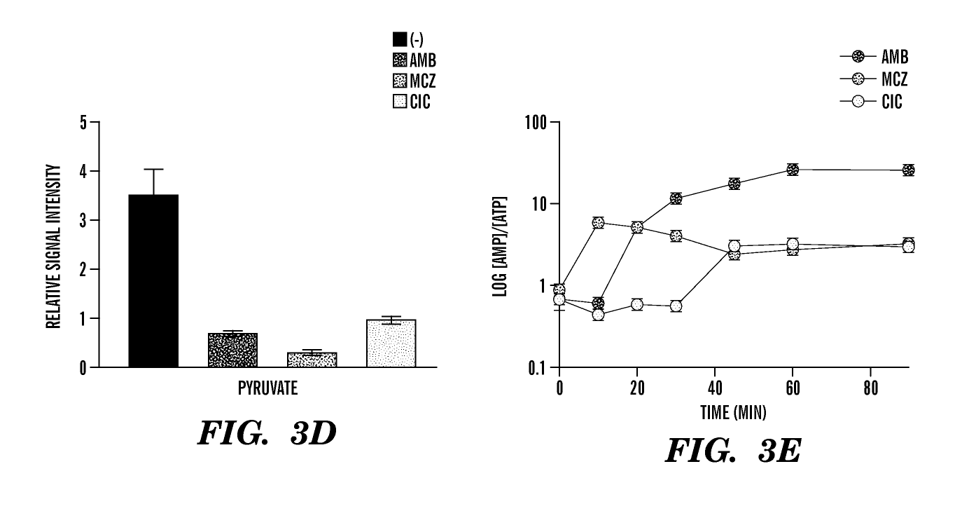

Glucose was the most highly induced metabolite, found to be greater than 600-fold more abundant in drug-treated cells (FIG. 3B). Other carbohydrates, such as fructose and mannose, were also significantly more abundant in all of the treatment groups compared to the control, as was the disaccharide trehalose (FIG. 3B). These results are consistent with the microarray data that identified the upregulation of polysaccharide biosynthesis as an inmportant transcriptional response to AF treatment.

Phenotypic analyses in S. cerevisiae suggested that cells may be switching from exclusively fermentative growth to mitochondrial respiration in response to AF treatment. Consistent with this result, 2,3-butanediol and glycerol, two major fermentative waste products (Gonzalez et al., 2000), were substantially reduced after AF treatment (FIG. 3C). Furthermore, pyruvate levels were dramatically lower in drug-treated samples (FIG. 3D), indicating possible consumption by the TCA cycle; of note, TCA cycle intermediates were also reduced in comparison to the untreated cells. These measurements, combined with our genetic data and measurements of mitochondrial activity and biogenesis, provide support for a common fungicidal mechanism of action that relies on reduced fermentation and induced ROS-producing mitochondrial respiration.

The dramatic elevation of intracellular sugars and the shift from fermentation to respiration suggests that the AF treatments may be increasing ATP consumption. Abundant nucleotides such as ATP are not accurately quantified using the metabolic platform selected for this study. It was therefore sought to measure ATP levels over time using HPLC. The AMP/ATP levels in lysates collected from cells treated with the three AFs were analyzed. The AF treatments elevated the AMP/ATP ratio by between 4- and 24-fold (FIG. 3E), through a large drop in ATP levels and a proportional rise in AMP levels. ATP levels are normally static, but can change dramatically under severe stress conditions that induce necrosis (Henriquez et al., 2008; Osorio et al., 2004). These results suggest that fungicide-stimulated sugar production induces a necrosis-like rapid consumption of ATP.

DNA Repair is a Critical Response to Antifungal-Dependent ROS Production.

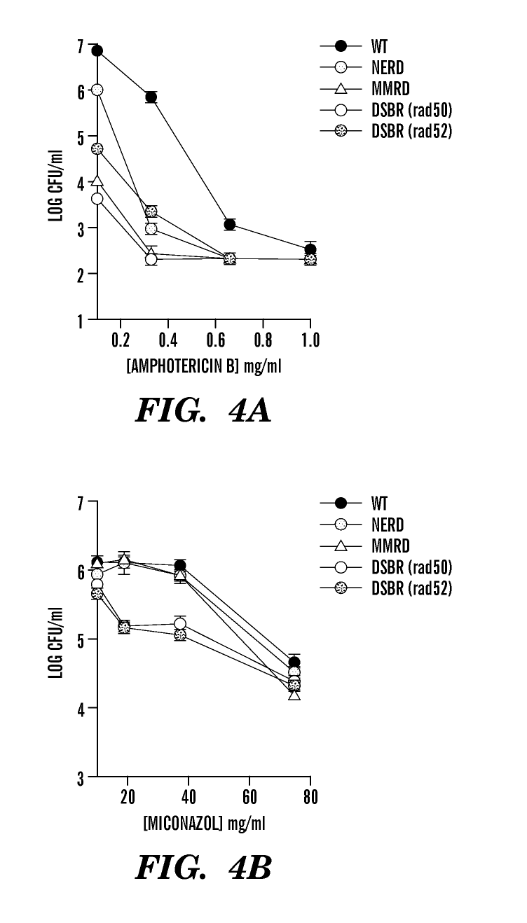

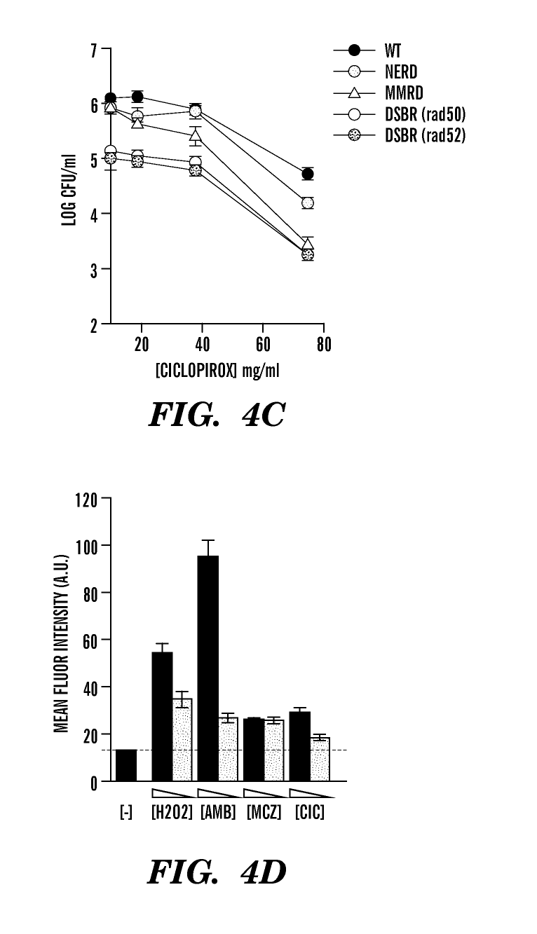

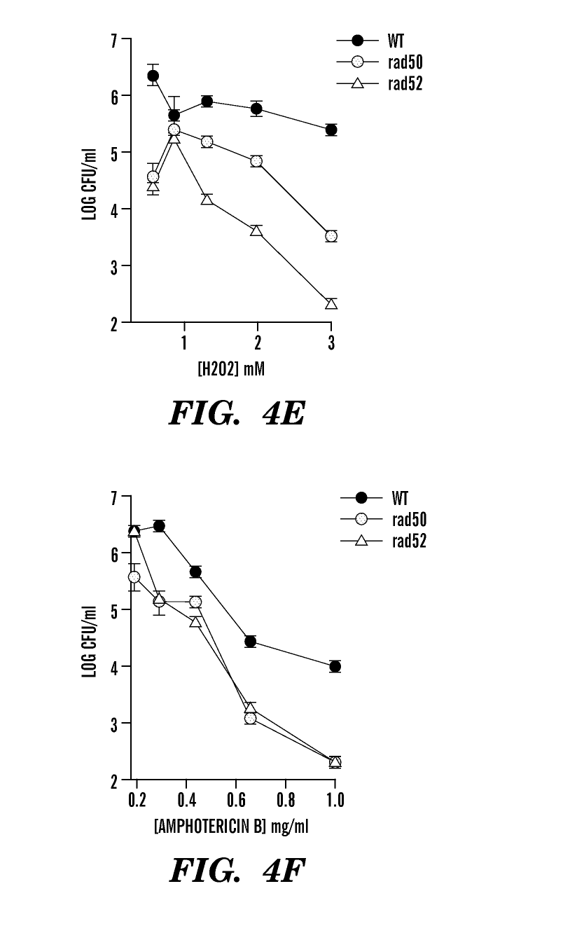

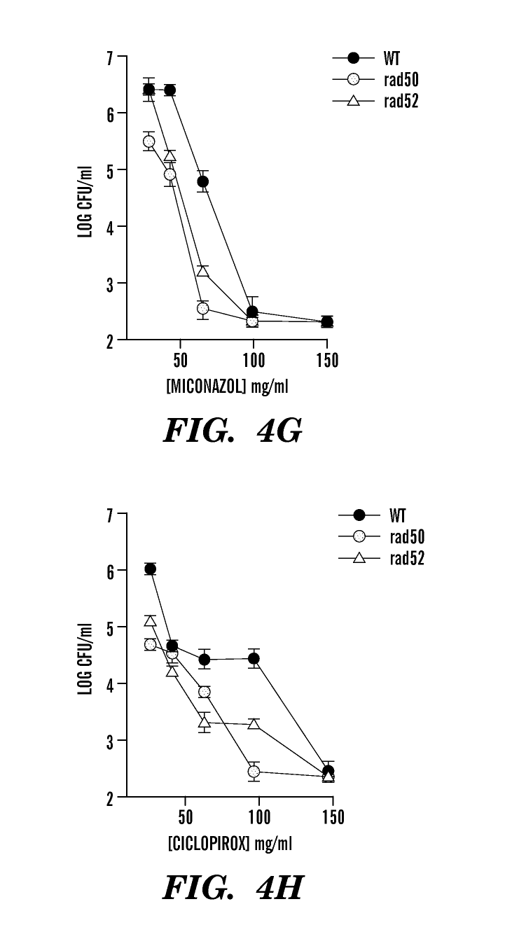

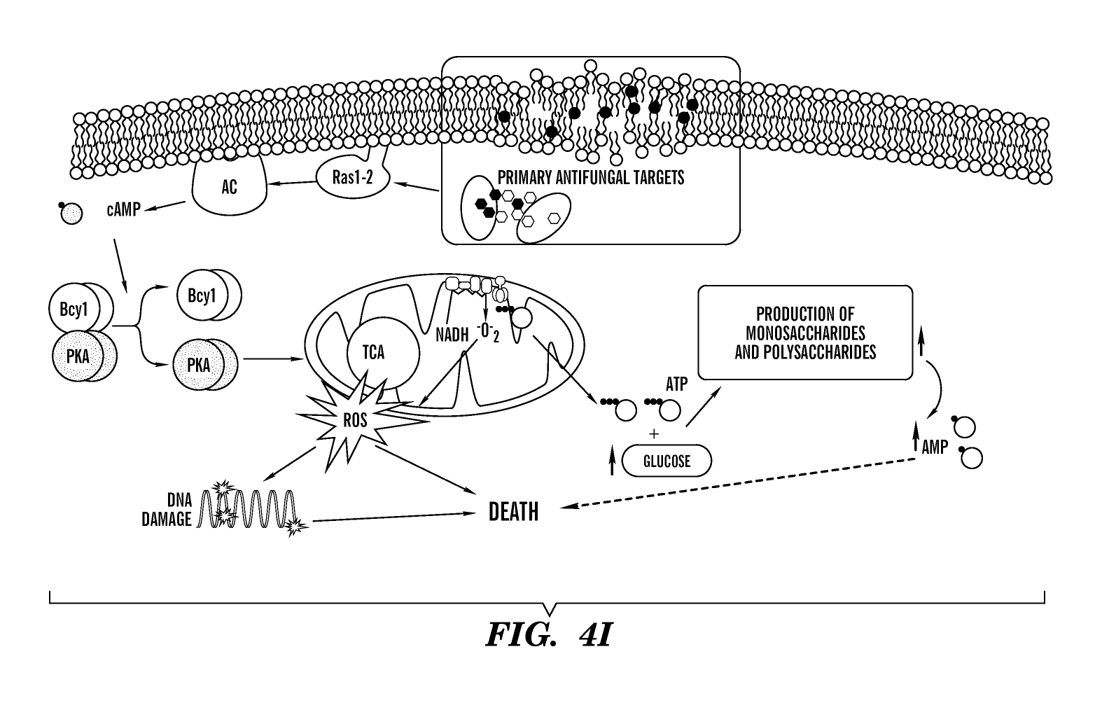

ROS damage multiple cellular targets, including membranes, proteins and DNA (Kohanski et al., 2007; Salmon et al., 2004). Recent work indicates that DNA repair, specifically, double-strand break repair (DSBR), plays a critical role in C. albicans resistance to oxidative damage (Legrand et al., 2007, 2008). The role of DNA repair in AF-induced cellular death was analyzed by testing the susceptibility of C. albicans strains with deletions of critical genes in nucleotide excision repair, mismatch repair, and DSBR. AMB, MCZ and CIC were used at a range of concentrations, and DSBR mutants were particularly susceptible to all three AFs, compared to wildtype (FIG. 4A-4C). Specifically, the minimal fungicidal concentrations for the DSBR mutants, rad50/rad50 and rad52/rad52, were reduced by as much as 10-fold compared to wildtype.

The terminal deoxynucleotidyl transferase dUTP nick-end labeling (TUNEL) assay was used to quantify the relative abundance of DSBs in C. albicans cells treated with AFs and H₂O₂. The TUNEL reagent fluorescently labels both double- and single-strand DNA breaks (Ribeiro et al., 2006). Treating cells with AFs and H₂O₂ elevated relative fluorescence as measured by flow cytometry (FIG. 4D), indicating a considerable induction of DNA breaks.

Rad50 and rad52 knockouts in S. cerevisiae were assayed to test whether the role of DNA damage in AF toxicity is consistent with the results from C. albicans. These mutants were considerably more susceptible to H₂O₂ and AFs than wildtype S. cerevisiae, although the differences were smaller than those seen with C. albicans. Together these data provide support for the role of DNA damage as a factor contributing to a common mechanism of AF action, and indicates that targeting DNA repair mechanisms can be an effective means for potentiating fungicidal activity.

Caspofungin Induces ROS Production and Metabolic Changes Consistent with the Common Mechanism of Antifungal-Induced Cellular Death.

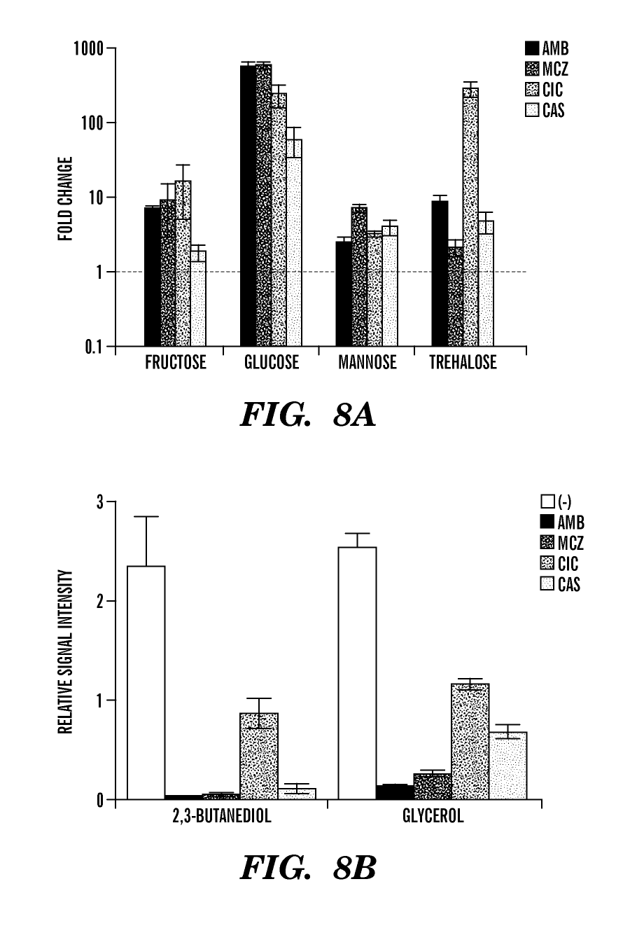

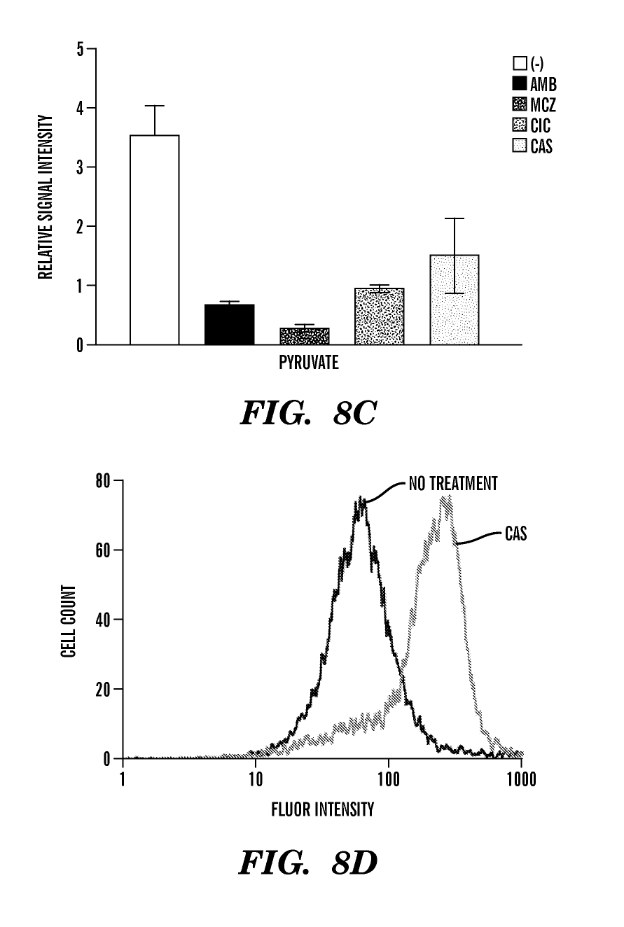

The key metabolic changes observed after AF treatment may act as a possible fingerprint to test whether other unrelated AFs may be acting through a similar common mechanism. Caspofungin belongs to a new class of AFs, termed echinocandins, that function by inhibiting cell wall synthesis in C. albicans (Denning, 2003). To test if AFs from this class of cidal drugs could induce metabolic changes comparable to the common mechanism of AF-induced cellular death in C. albicans, exponentially growing cells were treated with caspofungin and metabolic analyses conducted. The metabolic changes induced by caspofungin were similar to the changes induced by AMB, MCZ and CIC, respectively (FIGS. 8A-8C). Specifically, an elevation of sugars and a reduction in fermentative byproducts was found. Caspofungin treatment led to elevated ROS levels in C. albicans (FIG. 8D). These data indicate that caspofungin acts via a similar common mechanism.

### DISCUSSION

In this work, a systems biology approach was utilized to study the response of fungal cells to AF treatment. Despite divergent primary modes of action, fungicides from three distinct classes induce a common signaling and metabolic cascade that leads to ROS-dependent cellular death (FIG. 41). Without wishing to be bound by theory, the data indicate that cellular changes and damage initiated through interaction with primary targets of AFs results in activation of a stress-like response that includes signaling through the RAS/PKA pathway. Through this and possibly other pathways, cells activate mitochondrial activity and shift from fermentation to respiration in response to AFs. This abrupt induction of mitochondrial activity leads to the overproduction of toxic ROS in a manner that depends on the TCA cycle and ETC. Additionally, AF treatment leads to a buildup of monosaccharides and disaccharides, including glucose and trehalose. Both the import and synthesis of these sugars are energetically expensive, requiring the consumption of ATP and the production of AMP. It is likely that these metabolic changes lead to altered respiration and the overproduction of ROS by dysfunctional mitochondria, ultimately resulting in cell death (FIG. 4I).

Under various stress conditions, low-level ROS production can activate multiple protective responses including the upregulation of antioxidant enzymes and the induction of beneficial mutations (Belenky and Collins, 2011; Gems and Partridge, 2008; Kohanski et al., 2010). However, if ROS production goes above a certain threshold, it no longer serves a protective role and instead induces cellular death. Thus it is likely that many lethal challenges, including fungicides, function by highjacking natural stress response mechanisms to induce ROS production above this threshold.

### Experimental Procedures

Fungal Strains and Media.

S. cerevisiae strains used in this work were derivatives of BY4742, created as part of the Deletion Consortium (Winzeler et al., 1999). Deletion Consortium strains with reported phenotypes were verified by PCR. Stains PAB202 (BY4742 cit1Δ::kanMX4 cit3Δ::URA3), PAB205 (BY4742 cit3Δ::kanMX4 cit2Δ::LEU2) and PAB208 (PAB202 cit2Δ::LEU2) were created by direct transformation of PCR products as previously described (Brachmann et al., 1998). S. cerevisiae rad50 and rad52 knockouts were derivatives of MKP-0 and generously provided to us by Dr. Simone Moertl (Steininger et al., 2010). Wildtype C. albicans strain, SC5314, was used in metabolomic profiling and HPF measurements. C. albicans DNA repair mutants used in this work were derivatives of DKCa39, generously provided by Dr. David T. Kirkpatrick. A complete list of DKCa strains used in this study is provided. Synthetic dextrose complete (SDC) media and synthetic complete media with 2% acetate (pH 6.5) were prepared as previously described (Burke et al., 2000; Wickerham, 1946).

Fungicidal Killing and Fluorescent Dye Assays.

Overnight yeast cultures were diluted into the indicated media and grown to an OD600 of 0.2, at which point the AFs were added. Colony forming units (CFU) were measured by plating six serial dilutions onto YPD agar plates. A more detailed description is included elsewhere herein.

S. cerevisiae Microarrays.

Yeast cells were incubated in 25 ml of SDC and grown in 250 ml flasks at 30° C. and 300 rpm. AFs were added at an OD600 of 0.2. Cells were harvested after treatment, and their RNA was isolated and processed as previously described (Schmitt et al., 1990). The complete microarray analysis procedures are described elsewhere herein.

Metabolomic Profiling.

C. albicans cells were lysed and assayed by Metabolon Inc. (Durham, USA) as previously described (Shakoury-Elizeh et al., 2010). ATP and AMP were extracted as previously described (Walther et al., 2010) and analyzed by HPLC. This procedure is more fully described elsewhere herein.

Antifungal Drugs.

AF drugs were resuspended at 250 times the working concentration in DMSO (CIC was solubilized in ethanol) and frozen at 80° C. in one-time-use aliquots. DMSO was added to cells at 0.4% as the no-treatment control. To account for observed potency differences between different lots of AF drugs, each set of experiments was performed using frozen stocks from the same lot. Each AF drug was titrated against S. cerevisiae and C. albicans in order to identify the minimal fungicidal concentration. This drug concentration was used for the phenotypic assays in order to achieve maximal sensitivity for resistant mutants.

Fungicidal Killing and Fluorescent Dye Assays.

Overnight yeast cultures were diluted into the indicated media and grown to an OD600 of 0.2, at which point the AFs were added. Colony forming units (CFU) were measured by plating six serial dilutions onto YPD agar plates. Cells treated with MCZ were washed before serial dilution to stop growth inhibition associated with high concentrations of MCZ. CFU were counted after three days of incubation on agar plates. When indicated, thiourea (Fluka) was added 30 min prior to the addition of AFs. HPF (10 μM) (Invitrogen) and Mito Tracker (1 μM) (Invitrogen) dyes were added to PBS-washed cells at the indicated time points and incubated for 30 min prior to flow cytometry measurements, which were taken on the FACSCalibur (Becton-Dickinson). All experiments described in this section were conducted in 0.5 ml of SDC in 24-well plates incubated at 900 rpm and 37° C. for C. albicans and 30° C. and 300 rpm for S. cerevisiae. TUNEL assays were conducted as previously described (Ribeiro et al., 2006) and are described in detail in the TUNEL section of the Experimental Procedures.

High-Throughput 96-Well Screen.

To identify genes critical to AF action, the AF sensitivity of single-gene knockouts identified through the common transcriptional analysis were identified. In the first pass-through, 56 testable genes were identified. This set was later expanded to include targets identified through phenotypic analysis and metabolomic profiling, for a total of 84 target genes (Table 2). The initial 56 targets were tested using a rapid high-throughput 96-well assay with AMB and MCZ to identify single-gene knockouts with elevated resistance to AFs (Table 2). Knockouts that were more resistant to at least one of the AFs were tested individually using the 24-well colony forming (CF) assay. Target genes selected after the initial 56 strains were assayed individually using the 24-well CF assay.

The first 56 S. cerevisiae strains were acquired from the deletion library and stored in 96-well plates in replicates of three. To start the assay, the plates were defrosted and 10 μl of stored cells were inoculated into 100 μl of fresh synthetic dextrose complete (SDC) media for 16 hours. These overnight cultures were diluted into a fresh 96-well plate with 100 μl of SDC to achieve an OD600 of approximately 0.2. Colony forming units (CFU) in each well were measured at the start of the assay and after 8 hours of incubation. The AF susceptibility of each strain was compared to that of wildtype. Strains with significant susceptibility differences from wildtype were assayed in 0.5 ml of SDC media using the 24-well plate CF assay described above.

Metabolomic Profiling.

C. albicans cells were incubated in 100 ml of SDC in 250 ml flasks at 37° C. and 300 rpm. Cells were exposed to AFs at an OD600 of 0.2, and cellular pellets from 100 ml of media were collected after 1.5 h of treatment. Cells were lysed and assayed by Metabolon Inc. (Durham, USA) as previously described (Evans et al., 2009; Shakoury-Elizeh et al., 2010).

ATP and AMP Measurements.

To quantitate the cellular AMP/ATP ratio, C. albicans cells were incubated in 25 ml of SDC in 250 ml flasks at 37° C. and 300 rpm. AF drugs were added at an OD600 of 0.2, and 2 ml of cells were collected every 10 min for the first 90 min of incubation. Cells were lysed and nucleotides were extracted using boiling buffered methanol as previously described (Walther et al., 2010). Lysates were desiccated and resuspended in 60 ml of water prior to HPLC analysis. Nucleotides were separated isocratically in 100 mM sodium phosphate pH 6.5 on a ZORBAX SIL SAX 70 Å Sum, 4.6×150 mm column (Agilent, Santa Clara Calif.).

S. cerevisiae Microarrays.

Yeast cells were incubated in 25 ml of SDC and grown in 250 ml flasks at 30° C. and 300 rpm. AFs were added at an OD600 of 0.2.

Yeast cells were incubated in 25 ml of SDC and grown in 250 ml flasks at 30° C. and 300 rpm. Antifungals were added at an OD600 of 0.2. The rate of yeast killing by MCZ and CIC, respectively, was reduced by the transition from the 0.5 ml culture to 25 ml cultures. Rather than increasing drug concentration to match 0.5 ml killing levels, CIC and MCZ samples were collected after extending incubation times to achieve killing levels comparable to the 0.5 ml experiments. RNA from untreated cells was collected at 0, 0.5, 1 and 2 hours after treatment. RNA from AMB-treated cells was collected at 0.5, 1 and 2 hours after treatment. RNA from MCZ-treated cells was collected at 2, 5 and 8 hours after treatment. Cellular RNA was isolated and processed as previously described (Schmitt et al., 1990).

The effect of AF treatment on S. cerevisiae global gene expression was assayed using Affymetrix yeast microarrays (Affymetrix GeneChip Yeast Genome 2.0 array). The collected microarray data were added to a compendium of publicly available microarrays (obtained from the Gene Expression Omnibus) for a total of 536 chips. The set of expression profiles was normalized as a batch with RMA Express (Bolstad et al., 2003). The standard deviation of the expression of each gene was calculated across the entire compendium of expression profiles, allowing us to calculate a z-scale difference between a treatment and a control condition (no-treatment) using the formula:

$$Δ {z}_{exp} = \frac{{X}_{exp} - {X}_{ctr}}{σ}$$

This allowed the determination of gene expression changes in units of standard deviation, which is a form of the z-test. For each of the time points in each treatment, the z-score difference was converted into p-values and the sets of up- and down-regulated genes (p<0.05) were identified. The set of differentially expressed genes was merged across all time points, resulting in a global set of differentially expressed genes in AF-treated cells as compared to untreated cells. A common set of genes differentially expressed in response to all three antifungal drug treatments was determined by the intersection of the three distinct gene sets (FIGS. 1D and 1E).

TUNEL Assay.

DNA strand breaks were fluorescently labeled with TUNEL reagent from the “In Situ Cell Death Detection Kit”, Roche (Mannheim, Germany). After 2 hours of treatment 5 ml of OD 0.2 cells were fixed with 3.7% formaldehyde for 30 min at room temperature, and then washed with digestion buffer consisting of 10 mM Mes pH 6.5 and 1 M sorbitol. Cell were digested for 45 min at 30° C. with 2 U of Yeast Lytic Enzyme, MP Biomedicals, (Irvine, Calif.) in 50 μl of digestion buffer. Cells were then washed in PBS with 1 M sorbitol and resuspended in permeabilization solution consisting of 0.1% Triton X-100 and 0.1% sodium citrate and 1 M sorbitol for 10 min at room temperature. Cell were again washed in PBS with 1 M sorbitol and labeled for 60 min with 10 μl of TUNEL reaction mixture. Cells were then again washed in PBS with 1 M sorbitol and resuspended in sheath fluid. Mean relative florescence was measured by flow cytometry on the FACSCalibur (Becton-Dickinson).

TABLE 1
S. cerevisiae Strains Tested as Part of this Work. This table describes the single-deletion
yeast strains tested as part of this work. The first 56 strains were tested in a high-throughput
96-well format. The three right-most columns refer to results from the colony forming
assays conducted in 24-well plates and described in the main text. ″Resistant″ or ″Sensitive″
indicate at least a 5-fold CFU difference from wildtype at the final time point.

| Strain | | 96 Well | 96 Well | | | |
|---|---|---|---|---|---|---|
| Name | Gene | Screen AMB | Screen MCZ | CFU AMB | CFU CIC | CFU MCZ |
| PAB101 | WT | Normal | Normal | Normal | Not Tested | Not Tested |
| PAB102 | HSP104 | Resistant | Normal | Normal | Not Tested | Not Tested |
| PAB103 | SDH2 | Resistant | Resistant | Resistant | Resistant | Resistant |
| PAB104 | FRE8 | Resistant | Normal | Resistant | Not Tested | Not Tested |
| PAB105 | CHA4 | Resistant | Normal | Resistant | Not Tested | Not Tested |
| PAB106 | FMS1 | Resistant | Normal | Resistant | Normal | Not Tested |
| PAB107 | NDE1 | Resistant | Resistant | Resistant | Resistant | Resistant |
| PAB108 | ALD3 | Resistant | Normal | Normal | Not Tested | Not Tested |
| PAB109 | GAD1 | Normal | Normal | Not Tested | Not Tested | Not Tested |
| PAB110 | PGM3 | Normal | Normal | Not Tested | Not Tested | Not Tested |
| PAB111 | PYK2 | Resistant | Resistant | Not Tested | Not Tested | Not Tested |
| PAB112 | MNE1 | Normal | Normal | Resistant | Resistant | Resistant |
| PAB113 | FUM1 | Normal | Resistant | Resistant | Resistant | Resistant |
| PAB114 | TPK2 | Normal | Normal | Resistant | Normal | Resistant |
| PAB115 | ATG29 | Resistant | Normal | Not Tested | Not Tested | Not Tested |
| PAB116 | ISU1 | Resistant | Normal | Normal | Not Tested | Not Tested |
| PAB117 | CYC7 | Normal | Normal | Normal | Not Tested | Not Tested |
| PAB118 | ARN1 | Normal | Normal | Not Tested | Not Tested | Not Tested |
| PAB119 | CIT2 | Resistant | Resistant | Resistant | Resistant | Resistant |
| PAB120 | HSP30 | Normal | Resistant | Normal | Not Tested | Not Tested |
| PAB121 | SDH1 | Normal | Resistant | Resistant | Resistant | Resistant |
| PAB122 | TPK3 | Resistant | Resistant | Resistant | Resistant | Resistant |
| PAB123 | ISU2 | Normal | Normal | Normal | Not Tested | Not Tested |
| PAB124 | TPK1 | Normal | Normal | Resistant | Resistant | Resistant |
| PAB125 | MID2 | Normal | Normal | Not Tested | Not Tested | Not Tested |
| PAB126 | SSK1 | Sensitive | Normal | Not Tested | Not Tested | Not Tested |
| PAB127 | HSP42 | Normal | Normal | Not Tested | Not Tested | Not Tested |
| PAB128 | PIG1 | Normal | Normal | Not Tested | Not Tested | Not Tested |
| PAB129 | ACO1 | Poor Growth | Poor Growth | Not Tested | Not Tested | Not Tested |
| PAB130 | UGA1 | Resistant | Normal | Not Tested | Not Tested | Not Tested |
| PAB131 | MTL1 | Normal | Normal | Not Tested | Not Tested | Not Tested |
| PAB132 | CIT3 | Resistant | Resistant | Resistant | Resistant | Resistant |
| PAB133 | ZWF1 | Normal | Normal | Not Tested | Not Tested | Not Tested |
| PAB134 | SUM1 | sensitive | Normal | Not Tested | Not Tested | Not Tested |
| PAB135 | NDI1 | Resistant | Normal | Resistant | Resistant | Resistant |
| PAB136 | PGM2 | Normal | Resistant | Not Tested | Not Tested | Not Tested |
| PAB137 | HFD1 | Normal | Normal | Not Tested | Not Tested | Not Tested |
| PAB138 | PUF2 | Normal | Normal | Not Tested | Not Tested | Not Tested |
| PAB139 | TDH1 | Sensitive | Normal | Not Tested | Not Tested | Not Tested |
| PAB140 | GUD1 | Sensitive | Sensitive | Not Tested | Not Tested | Not Tested |
| PAB141 | ATG15 | Sensitive | Normal | Not Tested | Not Tested | Not Tested |
| PAB142 | TDH2 | Sensitive | Normal | Not Tested | Not Tested | Not Tested |
| PAB143 | ATP1 | Sensitive | Normal | Not Tested | Not Tested | Not Tested |
| PAB144 | UGA2 | Sensitive | Normal | Not Tested | Not Tested | Not Tested |
| PAB145 | CIT1 | Resistant | Resistant | Resistant | Resistant | Resistant |
| PAB146 | SOLI | Normal | Normal | Not Tested | Not Tested | Not Tested |
| PAB147 | PAN6 | Normal | Normal | Not Tested | Not Tested | Not Tested |
| PAB148 | SOL4 | Normal | Normal | Not Tested | Not Tested | Not Tested |
| PAB149 | YAK1 | Sensitive | Sensitive | Sensitive | Not Tested | Not Tested |
| PAB150 | PBS2 | Sensitive | Sensitive | Not Tested | Not Tested | Not Tested |
| PAB152 | GPD1 | Sensitive | Sensitive | Not Tested | Not Tested | Not Tested |
| PAB153 | SHO1 | Sensitive | Sensitive | Sensitive | Not Tested | Not Tested |
| PAB154 | FET3 | Sensitive | Sensitive | Not Tested | Not Tested | Not Tested |
| PAB155 | FBP1 | Sensitive | Sensitive | Sensitive | Not Tested | Not Tested |
| PAB156 | COX6 | Sensitive | Sensitive | Not Tested | Not Tested | Not Tested |
| PAB157 | SKN7 | Not Tested | Not Tested | Not Tested | Not Tested | Not Tested |
| PAB158 | MSN4 | Not Tested | Not Tested | Normal | Not Tested | Not Tested |
| PAB159 | SSK1 | Not Tested | Not Tested | Not Tested | Not Tested | Not Tested |
| PAB161 | MSN2 | Not Tested | Not Tested | Normal | Not Tested | Not Tested |
| PAB162 | YAP1 | Not Tested | Not Tested | Normal | Not Tested | Not Tested |
| PAB163 | CIN5 | Not Tested | Not Tested | Normal | Not Tested | Not Tested |
| PAB164 | Pde2 | Not Tested | Not Tested | Normal | Not Tested | Not Tested |
| PAB165 | USV1 | Not Tested | Not Tested | Normal | Not Tested | Not Tested |
| PAB166 | CAD1 | Not Tested | Not Tested | Normal | Not Tested | Not Tested |
| PAB167 | RIM101 | Not Tested | Not Tested | Normal | Not Tested | Not Tested |
| PAB168 | RAS1 | Not Tested | Not Tested | Resistant | Resistant | Resistant |
| PAB169 | HYR1 | Not Tested | Not Tested | Resistant | Not Tested | Not Tested |
| PAB170 | CRZ1 | Not Tested | Not Tested | Normal | Not Tested | Not Tested |
| PAB171 | RAS2 | Not Tested | Not Tested | Resistant | Resistant | Resistant |
| PAB172 | XBP1 | Not Tested | Not Tested | Normal | Not Tested | Not Tested |
| PAB173 | SRV2 | Not Tested | Not Tested | Normal | Not Tested | Not Tested |
| PAB174 | TPS3 | Not Tested | Not Tested | Resistant | Not Tested | Not Tested |
| PAB175 | TPS2 | Poor Growth | Poor Growth | Sensitive | Not Tested | Not Tested |
| PAB176 | SOD1 | Poor Growth | Poor Growth | Not Tested | Not Tested | Not Tested |
| PAB177 | SOD2 | Not Tested | Not Tested | Normal | Not Tested | Not Tested |
| PAB178 | CTA1 | Not Tested | Not Tested | Normal | Not Tested | Not Tested |
| PAB179 | CTT1 | Not Tested | Not Tested | Normal | Not Tested | Not Tested |
| PAB180 | MET1 | Not Tested | Not Tested | Normal | Not Tested | Not Tested |
| PAB181 | TVP18 | Not Tested | Not Tested | Normal | Not Tested | Not Tested |
| PAB182 | PML39 | Not Tested | Not Tested | Normal | Not Tested | Not Tested |
| PAB183 | VPS9 | Not Tested | Not Tested | Normal | Not Tested | Not Tested |

TABLE 2
Changes in Expression of Select Genes

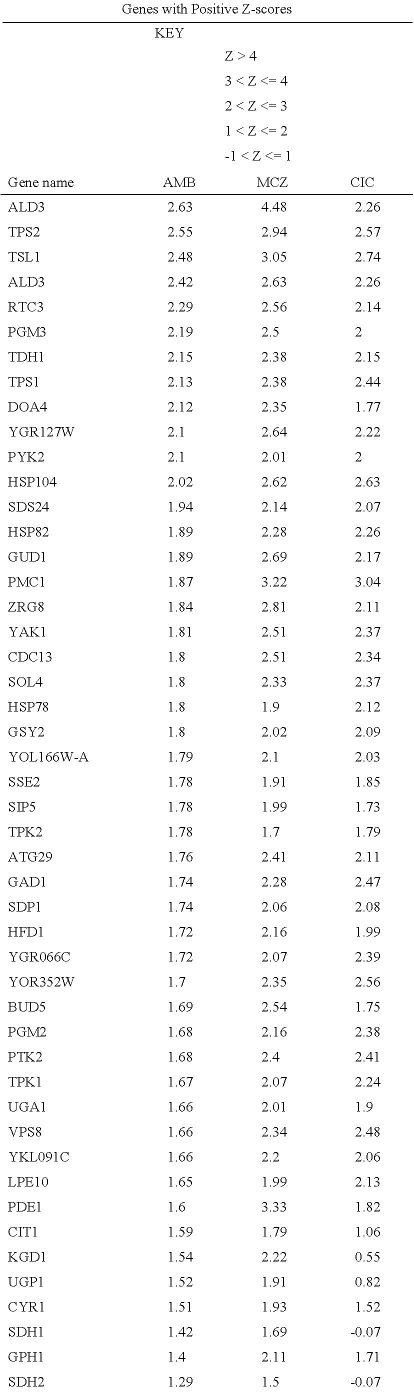

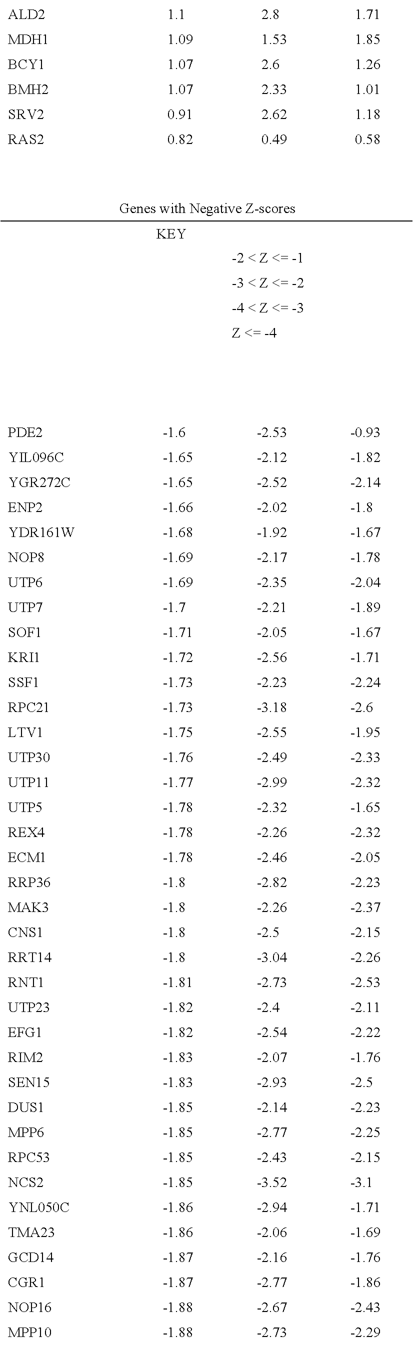

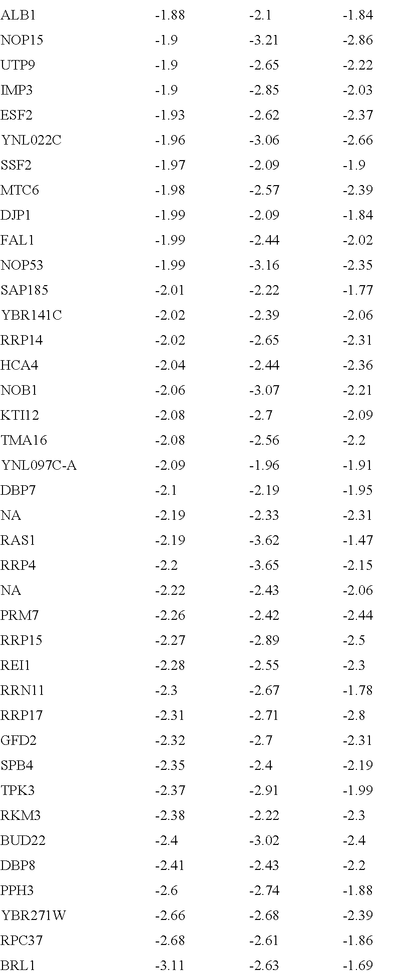

TABLE 3
DNA Damage Repair Deletion Strain List. A list
of C. albicans DKCa strains used in this work.

| Strain Name | Relevant Genotype |
|---|---|
| DKCa20 | rad10::CdHIS1/rad10::CdARG4 |
| DKCa33 | msh2::CdHIS1/msh2::CdARG4 |
| DKCa39 | WT |
| DKCa67 | rad50::CdHIS1/rad50::CdARG4 |
| DKCa96 | rad52::CdHIS1/rad52::CdARG4 |
| DKCa97 | rad52::CdHIS1/rad52::CdARG4 |

### REFERENCES

Arita, Y., Harkness, S. H., Kazzaz, J. A., Koo, H. C., Joseph, A., Melendez, J. A., Davis, J. M., Chander, A., and Li, Y. (2006). Mitochondrial localization of catalase provides optimal protection from H2O2-induced cell death in lung epithelial cells. Am J Physiol Lung Cell Mol Physiol 290, L978-986.
Belenky, P., and Collins, J. J. (2011). Microbiology. Antioxidant strategies to tolerate antibiotics. Science 334, 915-916.
Brachmann, C. B., Davies, A., Cost, G. J., Caputo, E., Li, J., Hieter, P., and Boeke, J. D. (1998). Designer deletion strains derived from Saccharomyces cerevisiae S288C: a useful set of strains and plasmids for PCR-mediated gene disruption and other applications. Yeast 14, 115-132.
Breitenbach, M., Laun, P., and Gimona, M. (2005). The actin cytoskeleton, RAS-cAMP signaling and mitochondrial ROS in yeast apoptosis. Trends Cell Biol 15, 637-639.
Burke, D., Dawson, D., and Stearns, T. (2000). Methods in Yeast Genetics (Cold Spring Harbor, N.Y., Cold Spring Harbor Press).
Chapman, K. B., Solomon, S. D., and Boeke, J. D. (1992). SDH1, the gene encoding the succinate dehydrogenase flavoprotein subunit from Saccharomyces cerevisiae. Gene 118, 131-136.
Chevtzoff, C., Yoboue, E. D., Galinier, A., Casteilla, L., Daignan-Fornier, B., Rigoulet, M., and Devin, A. (2010). Reactive oxygen species-mediated regulation of mitochondrial biogenesis in the yeast Saccharomyces cerevisiae. J Biol Chem 285, 1733-1742.
Dwyer, D. J., Camacho, D. M., Kohanski, M. A., Callura, J. M., and Collins, J. J. (2012). Antibiotic-induced bacterial cell death exhibits physiological and biochemical hallmarks of apoptosis. Molecular cell 46, 561-572.
Dwyer, D. J., Kohanski, M. A., and Collins, J. J. (2009). Role of reactive oxygen species in antibiotic action and resistance. Curr Opin Microbiol 12, 482489.
Dwyer, D. J., Kohanski, M. A., Hayete, B., and Collins, J. J. (2007). Gyrase inhibitors induce an oxidative damage cellular death pathway in Escherichia coli. Mol Syst Biol 3, 91.
Estruch, F. (2000). Stress-controlled transcription factors, stress-induced genes and stress tolerance in budding yeast. FEMS Microbiol Rev 24, 469486.
Evans, A. M., DeHaven, C. D., Barrett, T., Mitchell, M., and Milgram, E. (2009). Integrated, nontargeted ultrahigh performance liquid chromatography/electrospray ionization tandem mass spectrometry platform for the identification and relative quantification of the small-molecule complement of biological systems. Anal Chem 81, 6656-6667.
Foti, J. J., Devadoss, B., Winkler, J. A., Collins, J. J., and Walker, G. C. (2012). Oxidation of the guanine nucleotide pool underlies cell death by bactericidal antibiotics. Science 336, 315-319.
Gasch, A. P., Spellman, P. T., Kao, C. M., Carmel-Harel, O., Eisen, M. B., Storz, G., Botstein, D., and Brown, P. O. (2000). Genomic expression programs in the response of yeast cells to environmental changes. Mol Biol Cell 11, 4241-4257.
Gems, D., and Partridge, L. (2008). Stress-response hormesis and aging: “that which does not kill us makes us stronger”. Cell Metab 7, 200-203.
Gonzalez, E., Fernandez, M. R., Larroy, C., Sola, L., Pericas, M. A., Pares, X., and Biosca, J. A. (2000). Characterization of a (2R,3R)-2,3-butanediol dehydrogenase as the Saccharomyces cerevisiae YAL060W gene product. Disruption and induction of the gene. J Biol Chem 275, 35876-35885.
Gonzalez-Parraga, P., Sanchez-Fresneda, R., Zaragoza, O., and Arguelles, J. C. (2011). Amphotericin B induces trehalose synthesis and simultaneously activates an antioxidant enzymatic response in Candida albicans. Biochimica et biophysica acta 1810, 777-783.
Hardie, D. G., Salt, I. P., Hawley, S. A., and Davies, S. P. (1999). AMP-activated protein kinase: an ultrasensitive system for monitoring cellular energy charge. Biochem J 338 (Pt 3), 717-722.
Henriquez, M., Armisen, R I, Stutzin, A., and Quest, A. F. (2008). Cell death by necrosis, a regulated way to go. Curr Mol Med 8, 187-206.
Kohanski, M. A., DePristo, M. A., and Collins, J. J. (2010). Sublethal antibiotic treatment leads to multidrug resistance via radical-induced mutagenesis. Mol Cell 37, 311-320.
Kohanski, M. A., Dwyer, D. J., Hayete, B., Lawrence, C. A., and Collins, J. J. (2007). A common mechanism of cellular death induced by bactericidal antibiotics. Cell 130, 797-810.
Kohanski, M. A., Dwyer, D. J., Wierzbowski, J., Cottarel, G., and Collins, J. J. (2008). Mistranslation of membrane proteins and two-component system activation trigger antibiotic-mediated cell death. Cell 135, 679-690.
Leadsham, J. E., and Gourlay, C. W. (2010). cAMP/PKA signaling balances respiratory activity with mitochondria dependent apoptosis via transcriptional regulation. BMC Cell Biol 11, 92.
Leem, S. H., Park, J. E., Kim, I. S., Chae, J. Y., Sugino, A., and Sunwoo, Y. (2003). The possible mechanism of action of ciclopirox olamine in the yeast Saccharomyces cerevisiae. Mol Cells 15, 55-61.
Legrand, M., Chan, C. L., Jauert, P. A., and Kirkpatrick, D. T. (2007). Role of DNA mismatch repair and double-strand break repair in genome stability and antifungal drug resistance in Candida albicans. Eukaryot Cell 6, 2194-2205.
Legrand, M., Chan, C. L., Jauert, P. A., and Kirkpatrick, D. T. (2008). Analysis of base excision and nucleotide excision repair in Candida albicans. Microbiology 154, 2446-2456.
Lu, T. K., and Collins, J. J. (2009). Engineered bacteriophage targeting gene networks as adjuvants for antibiotic therapy. Proc Natl Acad Sci USA 106, 4629-4634.
Noubhani, A., Bunoust, O., Bonini, B. M., Thevelein, J. M., Devin, A., and Rigoulet, M. (2009). The trehalose pathway regulates mitochondrial respiratory chain content through hexokinase 2 and cAMP in Saccharomyces cerevisiae. J Biol Chem 284, 27229-27234.
Osorio, H., Moradas-Ferreira, P., Gunther Sillero, M. A., and Sillero, A. (2004). In Saccharomyces cerevisiae, the effect of H2O2 on ATP, but not on glyceraldehyde-3-phosphate dehydrogenase, depends on the glucose concentration. Arch Microbiol 181, 231-236.
Ostrosky-Zeichner, L., Casadevall, A., Galgiani, J. N., Odds, F. C., and Rex, J. H. (2010). An insight into the antifungal pipeline: selected new molecules and beyond. Nat Rev Drug Discov 9, 719-727.
Perrone, G. G., Tan, S. X., and Dawes, I. W. (2008). Reactive oxygen species and yeast apoptosis. Biochim Biophys Acta 1783, 1354-1368.
Phillips, A. J., Sudbery, I., and Ramsdale, M. (2003). Apoptosis induced by environmental stresses and amphotericin B in Candida albicans. Proc Natl Acad Sci USA 100, 14327-14332.
Ribeiro, G. F., Corte-Real, M., and Johansson, B. (2006). Characterization of DNA damage in yeast apoptosis induced by hydrogen peroxide, acetic acid, and hyperosmotic shock. Mol Biol Cell 17, 4584-4591.
Salmon, T. B., Evert, B. A., Song, B., and Doetsch, P. W. (2004). Biological consequences of oxidative stress-induced DNA damage in Saccharomyces cerevisiae. Nucleic Acids Res 32, 3712-3723.
Schmitt, M. E., Brown, T. A., and Trumpower, B. L. (1990). A rapid and simple method for preparation of RNA from Saccharomyces cerevisiae. Nucleic Acids Res 18, 3091-3092.
Shakoury-Elizeh, M., Protchenko, O., Berger, A., Cox, J., Gable, K., Dunn, T. M., Prinz, W. A., Bard, M., and Philpott, C. C. (2010). Metabolic Response to Iron Deficiency in Saccharomyces cerevisiae. Journal of Biological Chemistry 285, 14823-14833.
Soufi, B., Kelstrup, C. D., Stoehr, G., Frohlich, F., Walther, T. C., and Olsen, J. V. (2009). Global analysis of the yeast osmotic stress response by quantitative proteomics. Mol Biosyst 5, 1337-1346.
Steininger, S., Ahne, F., Winkler, K., Kleinschmidt, A., Eckardt-Schupp, F., and Moertl, S. (2010). A novel function for the Mre11-Rad50-Xrs2 complex in base excision repair. Nucleic Acids Res 38, 1853-1865.
Thevelein, J. M., and de Winde, J. H. (1999). Novel sensing mechanisms and targets for the cAMP-protein kinase A pathway in the yeast Saccharomyces cerevisiae. Mol Microbiol 33, 904-918.
Thevissen, K., Ayscough, K. R., Aerts, A. M., Du, W., De Brucker, K., Meert, E. M., Ausma, J., Borgers, M., Cammue, B. P., and Francois, I. E. (2007). Miconazole induces changes in actin cytoskeleton prior to reactive oxygen species induction in yeast. J Biol Chem 282, 21592-21597.
Tomas, E., Stanojevic, V., and Habener, J. F. (2011). GLP-1-derived nonapeptide GLP-1(28-36)amide targets to mitochondria and suppresses glucose production and oxidative stress in isolated mouse hepatocytes. Regul Pept 167, 177-184.
Touati, D., Jacques, M., Tardat, B., Bouchard, L., and Despied, S. (1995). Lethal oxidative damage and mutagenesis are generated by iron in delta fur mutants of Escherichia coli: protective role of superoxide dismutase. J Bacteriol 177, 2305-2314.
Walther, T., Novo, M., Rossger, K., Letisse, F., Loret, M. O., Portais, J. C., and Francois, J. M. (2010). Control of ATP homeostasis during the respiro-fermentative transition in yeast. Mol Syst Biol 6, 344.
Wickerham, L. J. (1946). A Critical Evaluation of the Nitrogen Assimilation Tests Commonly Used in the Classification of Yeasts. J Bacteriol 52, 293-301.
Winzeler, E. A., Shoemaker, D. D., Astromoff, A., Liang, H., Anderson, K., Andre, B., Bangham, R., Benito, R., Bocke, J. D., Bussey, H., et al. (1999). Functional characterization of the S. cerevisiae genome by gene deletion and parallel analysis. Science 285, 901-906.
Yan, L., Zhang, J. D., Cao, Y. B., Gao, P. H., and Jiang, Y. Y. (2007). Proteomic analysis reveals a metabolism shift in a laboratory fluconazole-resistant Candida albicans strain. J Proteome Res 6, 2248-2256.
Bolstad, B. M., Irizarry, R. A., Astrand, M., and Speed, T. P. (2003). A comparison of normalization methods for high density oligonucleotide array data based on variance and bias. Bioinformatics 19, 185-193.
Denning, D. W. (2003). Echinocandin antifungal drugs. Lancet 362, 1142-1151.
Evans, A. M., DeHaven, C. D., Barrett, T., Mitchell, M., and Milgram, E. (2009). Integrated, nontargeted ultrahigh performance liquid chromatography/electrospray ionization tandem mass spectrometry platform for the identification and relative quantification of the small-molecule complement of biological systems. Anal Chem 81, 6656-6667.
Legrand, M., Chan, C. L., Jauert, P. A., and Kirkpatrick, D. T. (2007). Role of DNA mismatch repair and double-strand break repair in genome stability and antifungal drug resistance in Candida albicans. Eukaryot Cell 6, 2194-2205.
Legrand, M., Chan, C. L., Jauert, P. A., and Kirkpatrick, D. T. (2008). Analysis of base excision and nucleotide excision repair in Candida albicans. Microbiology 154, 2446-2456.
Ribeiro, G. F., Corte-Real, M., and Johansson, B. (2006). Characterization of DNA damage in yeast apoptosis induced by hydrogen peroxide, acetic acid, and hyperosmotic shock. Mol Biol Cell 17, 4584-4591.
Schmitt, M. E., Brown, T. A., and Trumpower, B. L. (1990). A rapid and simple method for preparation of RNA from Saccharomyces cerevisiae. Nucleic Acids Res 18, 3091-3092.
Shakoury-Elizeh, M., Protchenko, O., Berger, A., Cox, J., Gable, K., Dunn, T. M., Prinz, W. A., Bard, M., and Philpott, C. C. (2010). Metabolic Response to Iron Deficiency in Saccharomyces cerevisiae. Journal of Biological Chemistry 285, 14823-14833.
Walther, T., Novo, M., Rossger, K., Letisse, F., Loret, M. O., Portais, J. C., and Francois, J. M. (2010). Control of ATP homeostasis during the respiro-fermentative transition in yeast. Mol Syst Biol 6, 344.

### Example 2

### cAMP Modulators Elevate Antifungal Activity

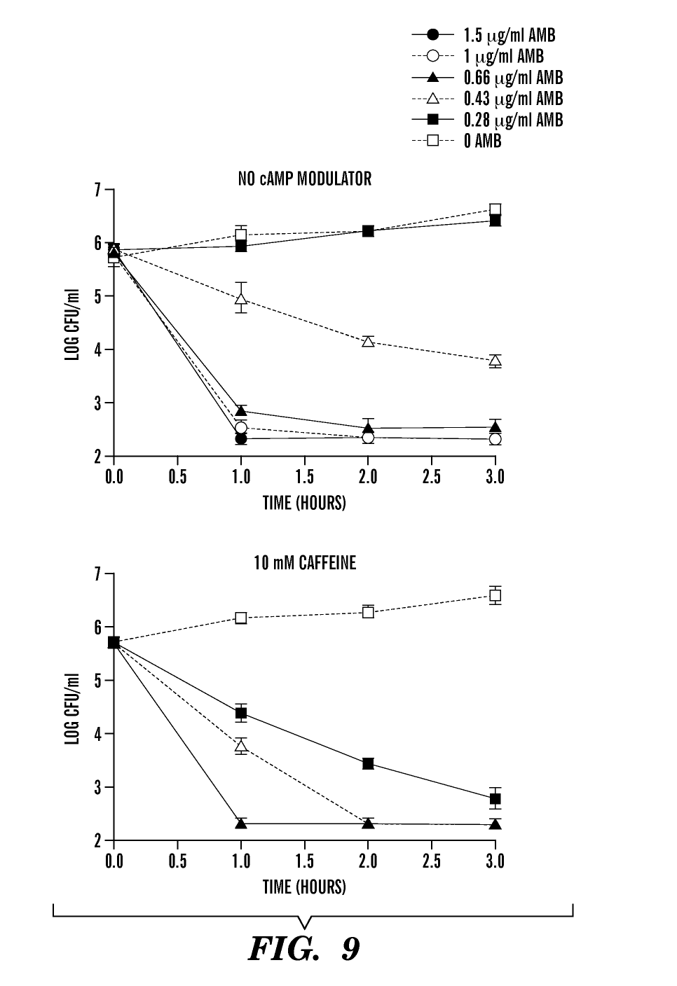

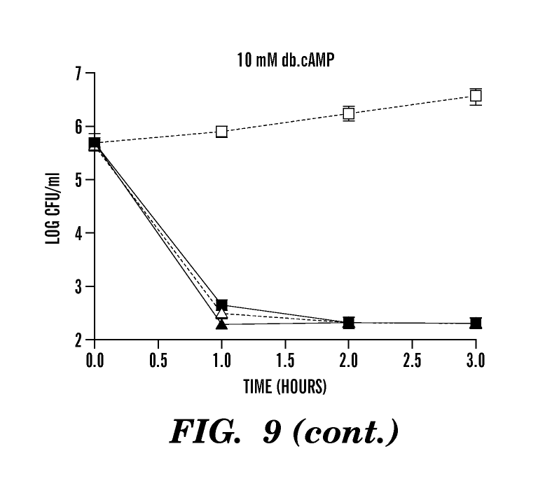

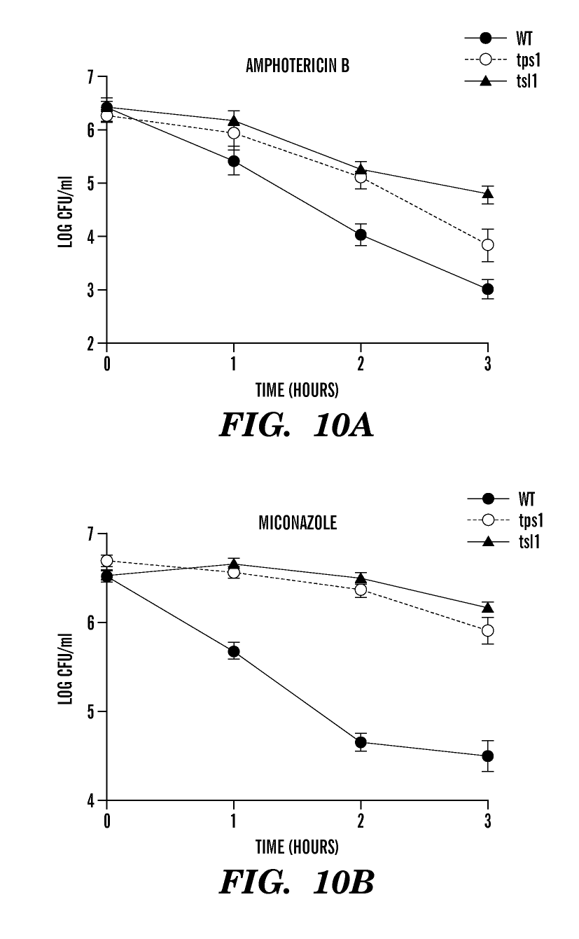

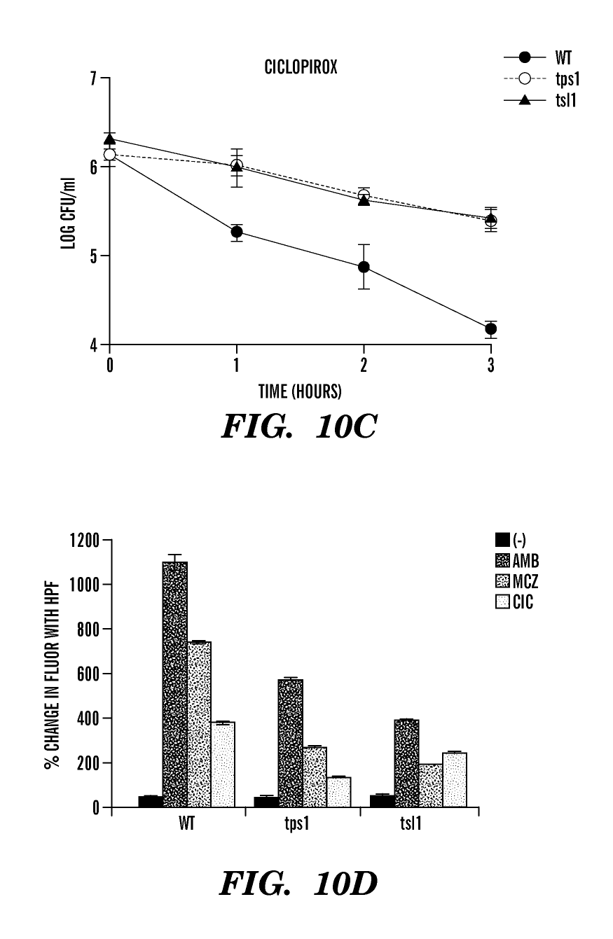

The model of the common death pathway induced by fungicidal drugs described above herein involves a signaling and metabolic cascade initiated by cAMP regulated RAS/PKA signaling. Based on this observation it was hypothesized that activating this pathway by inhibiting cAMP degradation with caffeine or providing dibutyryl-cAMP (db-cAMP) would elevate cellar death through activation of the Ras pathway. Consistent with this hypothesis pretreatment of Candida albicans with 10 mM db-cAMP or caffeine for 30 minutes improved AMB activity (FIG. 9).

### Example 3

It is demonstrated herein that yeast cells undergo dramatic metabolic changes following drug treatment, and thus provides methods to enhance the cidal impact of these changes by modulating the extracellular metabolic environment. One of the more dramatic metabolic changes observed was induction of intracellular glucose. Accordingly, it was hypothesized that reducing glucose levels below the high levels present in SDC media would apply additional metabolic pressure, sensitizing yeast cells to AF agents, i.e., reducing media glucose levels would force yeast cells to activate energetically costly gluconeogenesis in order to synthesize trehalose and maintain elevated intracellular glucose levels in response to AF treatment.

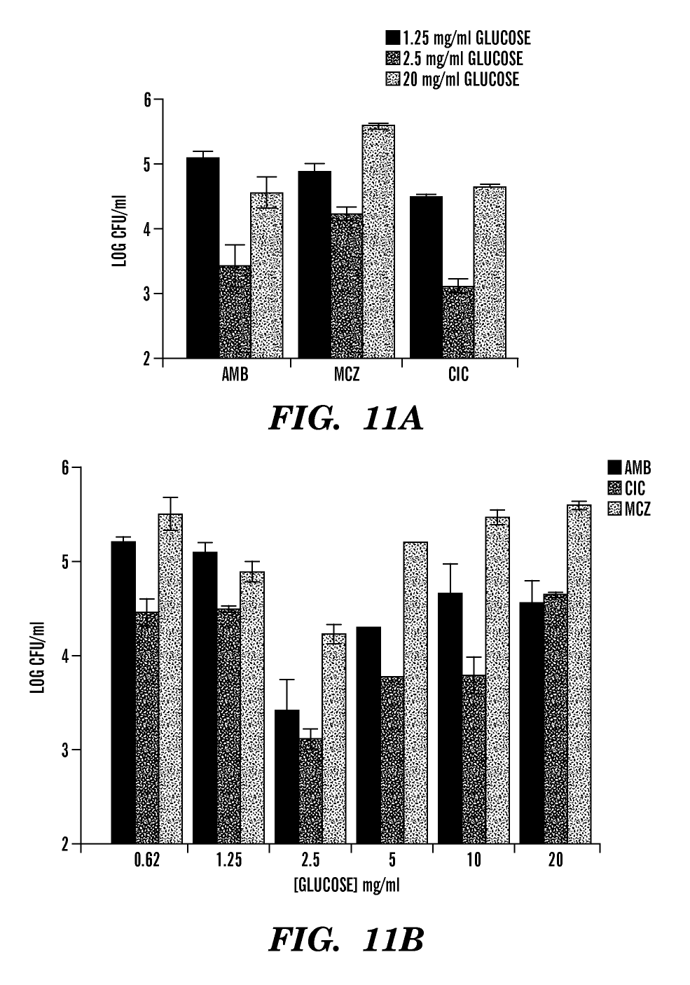

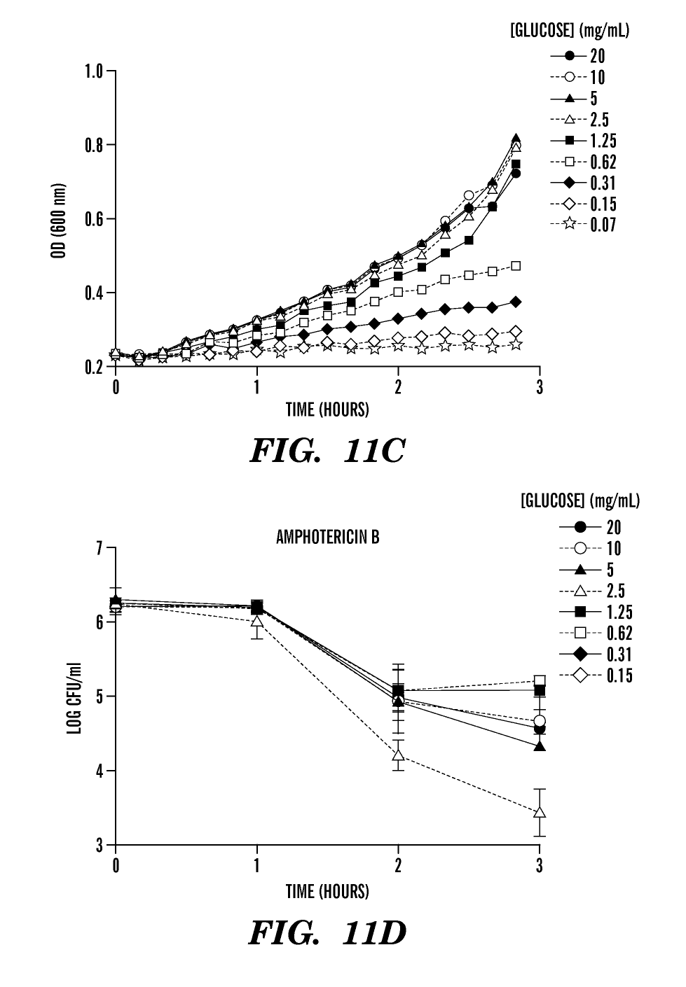

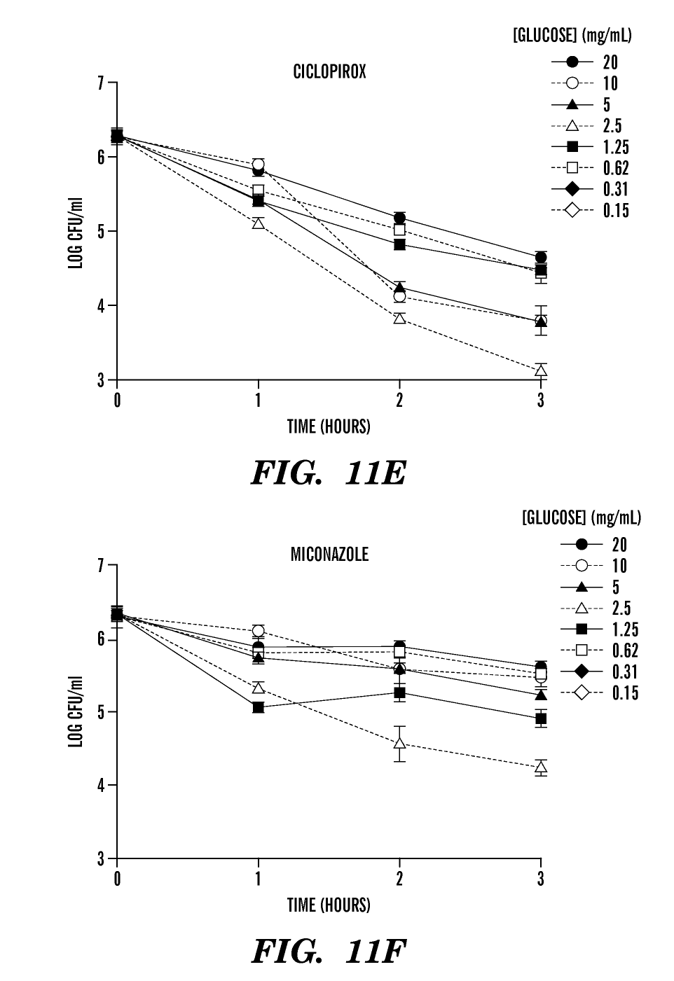

Exponential phase C. albicans was incubated in glucose-free SDC media for 60 min and then switched them to SDC containing glucose concentrations of between 20 mg/ml (normal SDC glucose levels) and 0.65 mg/ml glucose. Yeast cultured on the modified glucose SDC were then treated with AFs at the minimal fungicidal concentration. Reducing glucose levels to 2.5 mg/ml increased the fungicidal activity of all three antifungals (FIGS. 11A-11B). This finding is consistent with the hypothesis that lowering glucose would provide additional metabolic pressure and result in increased AF activity. Further reducing glucose levels below 2.5 mg/ml, actually blocked the potentiation effect (FIGS. 11A-11B). It was observed that yeast grown on extremely low glucose media have reduced growth rates when compared to glucose levels at or above 2.5 mg/ml. It is possible that cells grown in glucose levels below 2.5 mg/ml are less metabolically active and therefore less susceptible to drug treatment. Interestingly, the normal fasting blood glucose levels is close to 1 mg/ml (American Diabetes Association, 2009; Miller et al., 1956). Thus, these findings indicate that increasing blood glucose levels to approximately twice that of normal fasting glucose levels could enhance the cidal activity of AF drugs by as much as 100 fold.

1. A method for treating a fungal infection, comprising administering to a patient having a fungal infection and undergoing treatment with an antifungal agent, an effective amount of one or more potentiator compounds.

2-121. (canceled)

122. The method of claim 1, further comprising administering an effective amount of an antifungal agent.

123. The method of claim 1, wherein the subject is administered a pharmaceutical composition comprising one or more potentiator compounds and an antifungal agent.

124. The method of claim 1, wherein the potentiator compound is an agonist of the RAS/PKA pathway; an agonist of the TCA cycle or respiration; an inhibitor of DNA repair; cAMP or a mimetic or analog thereof; a cAMP modulator; a phosphodiesterase inhibitor, or glucose.

125. The method of claim 124, wherein the agonist of the RAS/PKA pathway is an agonist of RAS1; RAS2; Cyr1; Cdc25; Srv2; Tpk1; Tpk2; Tpk3; and orthologs and homologs thereof; or an inhibitor of Bcy1; Pde1; Pde2; or orthologs and homologs thereof.

126. The method of claim 124, wherein the inhibitor of Pde1 is IC224.

127. The method of claim 124, wherein the agonist of the TCA cycle or respiration is an agonist of Hap2; Hap3; Hap4; Hap5; Cit1; Cit2; Sdh1/2 or Orthologs and Homologs thereof.

128. The method of claim 124, wherein the potentiator compound modulates carbon source utilization or inhibits glucose utilization.

129. The method of claim 124, wherein the inhibitor of DNA repair is an inhibitor of double-strand break repair; an inhibitor of single-strand repair, or an inhibitor of direct reversal.

130. The method of claim 124, wherein the inhibitor of double-strand break repair is an inhibitor of Rad54; Rad51; Rad52; Rad55; Rad57; RPA; Xrs2; Mre1; Lif1; Nej1; or orthologs and homologs thereof.

131. The method of claim 130, wherein the inhibitor is wortmannin; rapamycin; vorinostat; 0⁶-BG; NVP-BEZ235; 2-(Morpholin-4-yl)-benzo[h]chomen-4-one; 1-(2-hydroxy-4-morpholin-4-yl-phenyl)-ethanone; Ku55933; NU7441; or SU11752.

132. The method of claim 124, wherein the cAMP mimetic or analog or modulator thereof is diburtyryl cAMP; caffeine; forskolin; 8-bromo-cAMP; phorbol ester, sclareline; cholera toxin (CTx); aminophylline; 2,4 dinitrophenol (DNP); norepinephrine; epinephrine; isoproterenol; isobutylmethylxanthine (IBMX); theophylline (dimethylxanthine); dopamine; rolipram; iloprost; prostaglandin E₁; prostaglandin E₂; pituitary adenylate cyclase activating polypeptide (PACAP); vasoactive intestinal polypeptide (VIP); (S)-adenosine; cyclic 3′,5′-(hydrogenphosphorothioate)triethyl ammonium; 8-bromoadenosine-3′,5′-cyclic monophosphate; 8-chloroadenosine-3′,5′-cyclic monophosphate; or N6,2′-O-dibutyryladenosine-3′,5′-cyclic monophosphate.

133. The method of claim 124, wherein the phosphodiesterase inhibitor is rolipram, mesembrine, drotaverine, roflumilast, ibudilast, piclamilast, luteolin, cilomilast, diazepam, arofylline, CP-80633, denbutylline, drotaverine, etazolate, filaminast, glaucine, HT-0712, ICI-63197, irsogladine, mesembrine, Ro20-1724, RPL-554, YM-976, sildenafil, vardenafil, tadalafil, udenafil, avanafil, sofyllin, pentoxifylline, acetildenafil, bucladesine, cilostamide, cilostazol, dipyridamole, enoximone, glaucine, ibudilast, icariin, inamrinone (formerly amrinone), lodenafil, luteolin, milrinone, mirodenafil, pimobendan, propentofylline, zardaverine, caffeine, theophylline, theobromine, 3-isobutyl-1-methylxanthine (IBMX), aminophylline, or paraxanthine.

134. The method of claim 1, wherein the potentiator is selected for its ability to increase ROS production or increase susceptibility to oxidative stress.

135. The method of claim 122, wherein the antifungal agent is a polyene; an imidazole; a triazole; a thiazole; an allylamine; or an echinocandin; or any salts or variants thereof.

136. The method of claim 1, wherein the fungal infection is an infection of skin or soft tissue; a superficial mycosis; a cutaneous mycosis; a subcutaneous mycosis; a vaginal mycosis; a systemic mycosis; or is an infected wound or burn.

137. The method of claim 1, wherein the infection is a surface wound, burn, or infection; infection of a mucosal surface; respiratory infection; infections of the eyes, ears, nose, or throat; or infection of an intestinal pathogen.

138. A method for inhibiting fungal growth, the method comprising contacting a fungal cell with an effective amount of one or more potentiator compounds and an effective amount of an antifungal agent.

139. A composition comprising a potentiator compound coformulated for use in inhibiting or treating a fungal infection, wherein the potentiator compound is an agonist of the RAS/PKA pathway; an agonist of the TCA cycle or respiration; an inhibitor of DNA repair, cAMP or a mimetic or analog thereof; a cAMP modulator, a phosphodiesterase inhibitor, or glucose.

140. A composition comprising an antifungal agent formulated in a glucose solution.


SEQUENCE LISTING (68 sequences)

SEQ ID NO: 1 - protein, 291 aa, Candida albicans
MLREYKLVVVGGGGVGKSALTIQLIQSHFVDEYDPTIEDSYRKQCTIDDQQVLLDVLDTAGQEEYSAMREQYMRTGEGFLLVYSINSLNSFQELNSFYDQILRVKDSDNVPVLVVGNKCDLEMERQVSYQDGLALANSFNCPFLETSAKQRINVEEAFYGLVRNINQYNAKIAEAEKQQQQQQQQQNANQQGQDQYGQQKDNQQSQFNNQINNNNNNTSAVNGGVSSDGIIDQNGNGGVSSGQANLPNQSQSQSQRQQQQQQQEPQQQSENQFSGQKQSSSKSKNGCCVIV

SEQ ID NO: 2 - protein, 320 aa, Candida albicans
MSLLLDSLHALTKNYDMCVIGSSNVGKSTLVLHYVYHHFDESLYDLDAVYTKRIITPDTNGKFREITIFESDFHIDMYTLTRERHVLNANTIVLVYAIDDYQSFTALEDYYERINQLRPSIPISVIASKLDLDTNREVSYYEGAEFAKRIGAVSFNECTRANVMGVNQAFESIANVAVKIQLDKDNTVPTVDNEIQQKENDQDSNITTTPTSTTTQSQTSNYSPQRSLQESIPVNNFTQYDNNPHQVNSNKNQFDAEQNTPLSTLTRESTMLTSDLNGSTPIAQTPSTSSRQRKTRLKQSSGPSAKSSQIHAENKCCIIT

SEQ ID NO: 3 - protein, 615 aa, Candida albicans
MSFLRRDKSKANFRDGSATGLEEPVSPTTHFSPNVPPPLDGNHGDHYHDPDSPRSSVVSLPQLIHNSATHHLKENYRGFHANKRPKGIANVPPLAQPIKPRFKKKSNSLLNKLIYSTKKEDDETATSGKESRSSSIISDEKRKSASSASSGSSRQKFRFSSFDSNLSTSSSSPPKDKKASVSDTVSDSSTVTASMSNMPTISIDLNLDEMHDIIKSPETPTPTTGLPTQKAEKKVSPTAIKNWQAPESWDVKAPIKKEEPHAPKIEEVAENDVAIDNVLEKKRLPVLYGTHQVPHVTNSKDIKSSHIIRVFKEDNTFTTILCPLETTTSELLAIVQKKFFLESTTNFQLSVCIGNCVKVLEDFEKPLKIQMGLLLLSGYTEEDKLRMLGREDLSFVCKFVVENIFLRSLTHDEEVLLSRNYVDVNISSLNLKNVPIIFHQHTYEIEKLNVANNPSIYLPLDFIQGCTSLAYVDFSHNGCSKFPNNLLEAPQLTHLNLEMNFLDEIPQRISCLSNLTNLKLSSNQLYSLPHSFSTLTNLKQLDLSSNYFDSYPEAVNKLTNLVELNFSYNDLSIIPESIANLINLQKLNLCTNKLSGTLPGYLSQLKCVKAFGYPI

SEQ ID NO: 4 - protein, 1558 aa, Candida albicans
MFQQPNYSDPGKHISTSSSVYPDSNSASPESSPRIPGAFEQIIIPRHKENPFRTNKPIEQQQQQQQQQQQQQQQQQQQQQQQQQQQQQQQHQQHQEHQANGHDEEQNRMPTEGSNQTFQSYMTARSGAYSFEEEDYENEVAEHQDQQSENGSIYSQNQEPQLRNAPSFSSINESIFHNSSKNTTLDTTENDETPLIDSSERYPDETPIVDDNGFLHEINHGEKDIIASEPIDSNEPTSLLPKQNLSKKFKRVSVALQNTRTSGESYNFRNVFQNNVDQANRTATQDNTAPKLERKSTYYRKLQEKKLQSSQEQQGTEVENMVISNDQHSEASSISTRASSTHTDTSGRDDEQDDNLVKKNSTNSSSNFVFSNNNLPSSYNLGGSKVNDDIPIRKDLIRENKAQVQQDAIDRERLQMYNNSTAMSEEGGLNIVYKDANTTSDDTYSKQPSPTKSEEKEDNLSDFFIRALHSFDSSTLQSKSDASICLSFEKNDIAFLHTIDESGWGEVTLLETLQTGWIPMNYFSLVVTSSDDEEDEDDSYEDNDDDDSNKIPNNHYLKPLLHACGQFLSNPLSHKDRRNKYTFSIRVINSIRDGVRLLLQQTDCLSRSNELVTKRPIVRKSRKSLLADWYNLMVKANEFKGTSNYNKIEILTLMVYQVSRKAVSFLEIWSAESKEIITRDHTGKKLYDDLNDCPLLRTPPLAKQRITEIHGVLFSYLGLIIGRLDLIEHNQIGCDMLETLAHQIILLLRELLFVSRTGSEYSKDKPRELDSSLDGLLSLVSDLVASVKNLVVRTVNETEEDRIHKFGGPQLNGARDYYYTPEGGELLQIASRMVKAISVTVASIRKLLEVTGDFKLSSERSYPDYSKMRIEPQEFIKKCSQGITKLKDMPIQQPNGATSTSNSGGIKAYKANRYSMIRAGKTGDLGLTENGVNYLQSLNNENGNGNNDVDELDGSSPFTSSAPEFKPFTSEGGNEPNNDSNINANINNELSVDSNGNLLGGSFKGLVYTLTNEDSPPEYFFVSTFFICFRSFSTGIDLIEELITRFQVGYVSNDINIDLKLKKRRKLVAKSIQLWMESYWNHEADYNLLTTLINFFNEGMSDYLPLDAIRLIDIGARLSSRPLVENRSRRIKDTKQLVNRSITNAAKLPRKTGSILGDEINRYSMIDSYSLSKINSTSSSNSTTSSSGHSASLPMPLGPTSTSKTSLLTSSQLETIEKVNLTYRAILGNSLCSQKYINSIKYIPLDLSVLMPNFYIIRSQSWVLSNYRPNLLDFHGLEIAKQLTLLESYIFCSIKPDELLNQNYTTKRAHLKLAPNVPLSLLFTNCLSGYVIESILQPNITIKLRISMVKIWLKIAISCLYLRNFNSLAAIITALESHLITRISSIWIKLEDKYTELYDYLSSIIHPEKNYRVYRNKLRNFLNSPMGLETPPIPIVPYFSLFLQDLTFINDGNPNYRKANTFLNQKLINIDKYLKITRIIADIECLQISYLDDPNIVGNGDGNSLMENGDDKPNYTINPVAPLQELILLELWKICQLNKTEEDRAWKLSCKIQPRDVS

SEQ ID NO: 5 - protein, 545 aa, Candida albicans
MSTEESQFNVQGYNIITILKRLEAATSRLEDITIFQEEANKNHYGVDSLTEKGTPKSRTVESSEATSDGKSLESTSFATFSEAPVEKSKLIVEFENFVESYVHPLVETSKKIDSLVGESAQYFYEAFVEQGKFLELVLQSQQPDMTDPALAKALEPMNAKCTKINELKDSNRKSPFFNHLSTFSESNAVFYWIGIPTPVSYITDTKDTVKFWSDRVLKEYKTKDQVHVEWVKQTLSVFDELKNYVKEYHTTGVAWNPKGKPFAEVVSQQTESAAKNSSSASGSAGGAAPPPPPPPPPATFFDDTEKDSENPSPASGGINAVFAELNQGANITSGLKKVDKSEMTHKNPELRKQPPVAPKKPAPPKKPSSLSGGVSSAPVKKPAKKELIDGTKWIIQNFTKADISDLSPITIEVEMHQSVFIGNCSDVTIQLKGKANAVSVSETKNVALVIDSLISGVDVIKSYKFGIQVLGLVPMLSIDKSDEGTIYLSQESIDNDSQVFTSSTTALNINAPKENDDYEELAVPEQFVSKVVNGKLVTQIVEHAG

SEQ ID NO: 6 - protein, 397 aa, Saccharomyces cerevisiae
MSTEEQNGGGQKSLDDRQGEESQKGETSERETTATESGNESKSVEKEGGETQEKPKQPHVTYYNEEQYKQFIAQARVTSGKYSLQDFQILRTLGTGSFGRVHLIRSRHNGRYYAMKVLKKEIVVRLKQVEHTNDERLMLSIVTHPFIIRMWGTFQDAQQIFMIMDYIEGGELFSLLRKSQRFPNPVAKFYAAEVCLALEYLHSKDIIYRDLKPENILLDKNGHIKITDFGFAKYVPDVTYTLCGTPDYIAPEVVSTKPYNKSIDWWSFGILIYEMLAGYTPFYDSNTMKTYEKILNAELRFPPFFNEDVKDLLSRLITRDLSQRLGNLQNGTEDVKNHPWFKEVVWEKLLSRNIETPYEPPIQQGQGDTSQFDKYPEEDINYGVQGEDPYADLFRDF

SEQ ID NO: 7 - protein, 444 aa, Candida albicans
MVNLLKKLHITKSHQSNQSNSDSNSLNSNTSMDNHQQQQQLQHQQYQQQFQQPQQQLYPGEQIVHPAAAQTGQNTTNVTAVSSSNITQSATSSLHSQQLQHVDVSKSAAEEAIRRSLLPERSTVSKGKYSLTDFSIMRTLGTGSFGRVHLVRSVHNGRYYAIKVLKKHQVVKMKQVEHTNDERRMLKLVEHPFLIRMWGTFQDSKNLFMVMDYIEGGELFSLLRKSQRFPNPVAKFYAAEVTLALEYLHSHDIIYRDLKPENILLDRNGHIKITDFGFAKEVSTVTWTLCGTPDYIAPEVITTKPYNKSVDWWSLGVLIFEMLAGYTPFYDSTPMKTYEKILAGKIHYPSFFQPDVIDLLTKLITADLTRRLGNLINGPADIRNHPWFSEVVWEKLLAKDIETPYEPPITAGVGDSSLFDHYPEEQLDYGSQGEDPYASYFLDF

SEQ ID NO: 8 - protein, 405 aa, Candida albicans
MTSMEPADTSIRSLNDINLQELANKQHQRNIYAQGSPTLEDSITSNGNDINNPNNNINKTNNNSNSSLSLTKKTSRHINKDTTTKGKYTLNDFQILRTLGTGSFGRVHLTRSIHNGRFYAMKVLKKQRVVQMKQIEHTNDERRMLKLAQHPFIIRMWGTFQDCHNLFMIMDYIEGGELFSLLRKSQRFPTPVAKFYAAEVFLAIEYLHSLDIIYRDLKPENILLDKNGHIKLTDFGFAKEVQDVTYTLCGTPDYIAPEVVATKPYNKSVDWWSFGILIFEMLTGYTPFYDPTPMKTYENILNGSITYPDYLPPDILDLLQKLIVKDLTQRLGNLQGGSDDVKNHPWFKEVIWERLLSRDIETPYEPPITSGVGDTSQFDRYPEDKDLDYGISGVEDPYRDQFQDF

SEQ ID NO: 9 - protein, 459 aa, Candida albicans
MSNPQQQFISDELSQLQKEIISKNPQDVLQFCANYFNTKLQAQRSELWSQQAKAEAAGIDLFPSVDHVNVNSSGVSIVNDRQPSFKSPFGVNDPHSNHDEDPHAKDTKTDTAAAAVGGGIFKSNFDVKKSASNPPTKEVDPDDPSKPSSSSQPNQQSASASSKTPSSKIPVAFNANRRTSVSAEALNPAKLKLDSWKPPVNNLSITEEETLANNLKNNFLFKQLDANSKKTVIAALQQKSFAKDTVIIQQGDEGDFFYIIETGTVDFYVNDAKVSSSSEGSSFGELALMYNSPRAATAVAATDVVCWALDRLTFRRILLEGTFNKRLMYEDFLKDIEVLKSLSDHARSKLADALSTEMYHKGDKIVTEGEQGENFYLIESGNCQVYNEKLGNIKQLTKGDYFGELALIKDLPRQATVEALDNVIVATLGKSGFQRLLGPVVEVLKEQDPTKSQDPTAGH

SEQ ID NO: 10 - protein, 426 aa, Candida albicans
MSFEITILGSSGGPLEGSTCSILLKPANISYHDIINDNLPDQVLCIDAGSGMGKLAEIIHQETTTKTSYCNFLQYYPDCETVSYYYHPNVTITTPFSNFQPGRPILHTQNIFNNLQNYLISHSHLDHVCSVVINSAGFNKNMSNKILYGSHYTINAMQQHLFNGKVWPNMPSFKIVNLNYLESNRSERIGIYTVKMFDLSHGEFNKLTEDKEDAQHHSNSNSNSNNIWGKRYDRRRSSITTIPQNTSGLIIKNSEALNHHYLSSAFLITLEVPCTIKEPPSSILVFGDFESDLTSKLSRNLFIWKSIAPLILRNQLKAIVLECSNCKEIAANELYGHLTPKLLIYELKQLAHECKQLDTATTSTEQPLLGLNVIVNHVKEPIADPNQESQLHDPRKRILAELNKLNEIEKLGCNISIALSGTSIIV

SEQ ID NO: 11 - protein, 571 aa, Candida albicans
MAEVLSLVDLEIPQVTDKYYKFDTFKHLICHLFKKTSTETDSNVPIVIIFPTNNDIPSRKTRSTTTTTTTTTTTNTSKLDNLPFSDKSLLIQFFFTHLNILMIQGENSDEGKLYQEISSAKELLTNRISRVGNWTGTTHFRYCRHENDCGLLNQHSKIAGIIPTMTYILNCNATRSEIATNQLIYLYRLMIEEINFIELLQDASTTRLSQLCYAVGHWSFPAHNLSNDDLVYCVYLMIDYAIKQVEGFDNIPLNELLAFIFIVRDTYKNGNPFHNFRHAVDVLQACFHFLIRLGSLPKFKQFVEDPKLDYTEVHDKHTVLIALQNNSSEEKASLNPIQTLGLLVAALGHDVGHPGTTNDFMIKFSAPTALLYNDRSVLESYHASLFINKVLRICWPDLLTCTIEEKSELTIRSLIISSILATDMGEHNEYVNRLKSFKTHNEILNHDNTVKLISALLIKCADISNVTRPLRVSAQWAMVLSREFAEVELLKSVIKKDIDLDFTKDLTYDDVPHELREILEIQPDIHKGQIFFINLFAENLFNSVSDLLPQLQYTCDIIMENKLFWLERAKK

SEQ ID NO: 12 - protein, 363 aa, Candida albicans
MTSLEDPNGQAQATYFQESVSTTGNSTPTVTQSNDPQHHHHHQQQQQQHHHHHVGSYYQDSPSSTYTYEVEGDYLENHPNVTSTSAEQGTGTMPTYVDNDNIHGGVGVGMGVETEEEDDDDTNGASIGNGSGSGGGSGGGEQPFYVNAKQYHRILKRRIARAKLEENLKIARTRKPYLHESRHKHAMRRPRGQGGRFLTAAEIAELEKAKQQQQQQQQQQQQQNEQSNQENNNENGNEIKKENQHVDDSNKIKNGDNNINKENIIENNKEEIDPNNVIIKKENNNNEVNHEMNNIENEQSSNEDFNQNNNNNINNNNTTISNNDNENGNTISSSKSNELQESIKNDDNNNNNNGNDLQEEEER

SEQ ID NO: 13 - protein, 105 aa, Candida albicans
MNQQNARDIELREQDRWLPIANVARIMKNTLPPTAKVSKDAKECMQECVSEFISFITSEASDKCLKEKRKTINGEDILYSMYDLGFENYAEVLKIYLAKYREARK

SEQ ID NO: 14 - protein, 554 aa, Saccharomyces cerevisiae
MTAKTFLLQASASRPRSNHFKNEHNNIPLAPVPIAPNTNHHNNSSLEFENDGSKKKKKSSLVVRTSKHWVLPPRPRPGRRSSSHNTLPANNTNNILNVGPNSRNSSNNNNNNNIISNRKQASKEKRKIPRHIQTIDEKLINDSNYLAFLKFDDLENEKFHSSASSISSPSYSSPSFSSYRNRKKSEFMDDESCTDVETIAAHNSLLTKNHHIDSSSNVHAPPTKKSKLNDFDLLSLSSTSSSATPVPQLTKDLNMNLNFHKIPHKASFPDSPADFSPADSVSLIRNHSLPTNLQVKDKIEDLNEIKFFNDFEKLEFFNKYAKVNTNNDVNENNDLWNSYLQSMDDTTGKNSGNYQQVDNDDNMSLLNLPILEETVSSGQDDKVEPDEEDIWNYLPSSSSQQEDSSRALKKNTNSEKANIQAKNDETYLFLQDQDESADSHHHDELGSEITLADNKFSYLPPTLEELMEEQDCNNGRSFKNFMFSNDTGIDGSAGTDDDYTKVLKSKKISTSKSNANLYDLNDNNNDATATNELDQSSFIDDLDEDVDFLKVQVF

SEQ ID NO: 15 - protein, 348 aa, Candida albicans
MNEDPQSEIMERYNESAYLRDDQLPQFDQNNDVIKEEQTHTQDHHHQHPSVSGSHSDELQDQDIHNVIEEHEDHVSTQNIVDEDELLAAQAAAEAAAAAQQQQPGDVFNNVAQGLSGKHRDMMMQYWQETINSIEHDEHDFKNHQLPLARIKKVMKTDEDVRMISAEAPILFAKGCDVFITELTMRAWIHAEENKRRTLQKSDIAAALTKSDMFDFLIDVVPREEEKPKKSNNNSSRSDSYVNNQQSPPIPETEEEVPHMLTQDVKHSHQDVLQQQMLQEQLLQQQLHQEVQQQLQQEVQRQLQEDVPQSDHQQASQREEDITNGAENANGHNDEATYENFNGYQNNY

SEQ ID NO: 16 - protein, 397 aa, Candida albicans
MRGIKGLVWEGSVLDPIEGIRFRGRTIPDIQKELPKAPGGEEPLPEALFWLLLTGEVPTDAQTKALSEEFAARSALPKHVEELIDRSPSHLHPMAQFSIAVTALESESQFAQAYAKGANKSEYWKYTYEDSIDLLAKLPTIAAKIYRNVFHDGKLPAAIDSKLDYGANLASLLGFGDNKEFVELMRLYLTIHSDHEGGNVSAHTTHLVGSALSSPFLSLAAGLNGLAGPLHGRANQEVLEWLFKLREELNGDYSKEAIEKYLWETLNSGRVVPGYGHAVLRKTDPRYTAQREFALKHMPDYELFKLVSNIYEVAPGVLTKHGKTKNPWPNVDSHSGVLLQYYGLTEQSFYTVLFGVSRAFGVLPQLILDRGIGMPIERPKSFSTEKYIELVKNINKA

SEQ ID NO: 17 - protein, 460 aa, Saccharomyces cerevisiae
MTVPYLNSNRNVASYLQSNSSQEKTLKERFSEIYPIHAQDVRQFVKEHGKTKISDVLLEQVYGGMRGIPGSVWEGSVLDPEDGIRFRGRTIADIQKDLPKAKGSSQPLPEALFWLLLTGEVPTQAQVENLSADLMSRSELPSHVVQLLDNLPKDLHPMAQFSIAVTALESESKFAKAYAQGISKQDYWSYTFEDSLDLLGKLPVIAAKIYRNVFKDGKMGEVDPNADYAKNLVNLIGSKDEDFVDLMRLYLTIHSDHEGGNVSAHTSHLVGSALSSPYLSLASGLNGLAGPLHGRANQEVLEWLFALKEEVNDDYSKDTIEKYLWDTLNSGRVIPGYGHAVLRKTDPRYMAQRKFAMDHFPDYELFKLVSSIYEVAPGVLTEHGKTKNPWPNVDAHSGVLLQYYGLKESSFYTVLFGVSRAFGILAQLITDRAIGASIERPKSYSTEKYKELVKNIESKL

SEQ ID NO: 18 - protein, 638 aa, Candida albicans
MFTKCSRQAVNRSGRFFSNSSITFQTIGRIKGTEANSKKYLQQKYVVIDHEYDCVVVGAGGAGLRAAFGLAESGFKTACISKLFPTRSHTVAAQGGINAALGNMHKDDWHWHFYDTVKGSDWLGDQDAIHYMTKEAPDSIYELEHFGVPFSRNDEGRIYQRAFGGQTKEFGKGGQAYRTCAVADRTGHALLHSLYGQALRHDCHFFIEFFAMDLLMQDGECVGVIAYNQEDGTIHRFRSHKTVIATGGYGRAYFSCTSAHTCTGDGYAMASRAGLPLQDLEFIQFHPSGIYGSGCLITEGARGEGGFLVNSEGERFMERYAPTAKDLACRDVVSRAITMEINEGRGVGSEKDHMYLQLSHLPAAVLKQRLPGISETAHIFAGVDVTKEPIPILPTVHYNMGGIPTNWQGEVLKKGTDGKDEVVPGLLACGEAACASVHGANRLGANSLLDLVVFGRAVSHTIRDNLTPGAPLHASPADLGKASIENLHRLRNAEGTKSTAEIRLDMQKTMQKGCAVFRTEETLEECVDNINEVDKSFANVKTTDRSMIWNSDLVETMELQNLLTCATQTAASALARKESRGSHSREDYPDRDDVNWWKHTLSYQNSVGSKVKLDYRDVIKTTLDTTDCQPVPPAVRKY

SEQ ID NO: 19 - protein, 263 aa, Candida albicans
MFRSILHQQKAVQFSVRSLATAAAEKAPRLKKFQIYRWNPDTPEVQPKMQTYEVDLNKCGPMVLDALLKIKNEQDATLTLRRSCREGICGSCAMNIGGRNTLACLCRIDQDESKDLKVYPLPHMFVVRDLVPDLTHFYKQYKSIEPYLQRESNPADGRENLQSIEDRAKLDGLYECILCACCSTSCPSYWWNQQQYLGPAVLMQAYRWLIDSRDQATANRKAMLQNSMSLYRCHTIMNCARTCPKGLNPGKAIAEIKKQLAFD

SEQ ID NO: 20 - protein, 188 aa, Saccharomyces cerevisiae
MKELLYYTFIETEVTGAFLVFREKTQNLVFASLGNDKLFLLGKVEGFLKKHEKQDTMYDLQELKEAETYKKSIENYTICLENKMPLPSGAIPFEFLFGTDFQRKVWNELLNVEHGHVVTYGDIAKRIGKPTAARSVGRACGSNNLALLVPCHRIVGSNRKLTGYKWSCKLKEQLLNNEKENSLSLSRL

SEQ ID NO: 21 - protein, 565 aa, Saccharomyces cerevisiae
MKRTVISSSNAYASKRSRLDIEHDFEQYHSLNKKYYPRPITRTGANQFNNKSRAKPMEIVEKLQKKQKTSFENVSTVMHWFRNDLRLYDNVGLYKSVALFQQLRQKNAKAKLYAVYVINEDDWRAHMDSGWKLMFIMGALKNLQQSLAELHIPLLLWEFHTPKSTLSNSKEFVEFFKEKCMNVSSGTGTIITANIEYQTDELYRDIRLLENEDHRLQLKYYHDSCIVAPGLITTDRGTNYSVFTPWYKKWVLYVNNYKKSTSEICHLHIIEPLKYNETFELKPFQYSLPDEFLQYIPKSKWCLPDVSEEAALSRLKDFLGTKSSKYNNEKDMLYLGGTSGLSVYITTGRISTRLIVNQAFQSCNGQIMSKALKDNSSTQNFIKEVAWRDFYRHCMCNWPYTSMGMPYRLDTLDIKWENNPVAFEKWCTGNTGIPIVDAIMRKLLYTGYINNRSRMITASFLSKNLLIDWRWGERWFMKHLIDGDSSSNVGGWGFCSSTGIDAQPYFRVFNMDIQAKKYDPQMIFVKQWVPELISSENKRPENYPKPLVDLKHSRERALKVYKDAM

SEQ ID NO: 22 - protein, 376 aa, Saccharomyces cerevisiae
MSYKFGKLAINKSELCLANVLQAGQSFRWIWDEKLNQYSTTMKIGQQEKYSVVILRQDEENEILEFVAVGDCGNQDALKTHLMKYFRLDVSLKHLFDNVWIPSDKAFAKLSPQGIRILAQEPWETLISFICSSNNNISRITRMCNSLCSNFGNLITTIDGVAYHSFPTSEELTSRATEAKLRELGFGYRAKYIIETARKLVNDKAEANITSDTTYLQSICKDAQYEDVREHLMSYNGVGPKVADCVCLMGLHMDGIVPVDVHVSRIAKRDYQISANKNHLKELRTKYNALPISRKKINLELDHIRLMLFKKWGSYAGWAQGVLFSKEIGGTSGSTTTGTIKKRKWDMIKETEAIVTKQMKLKVELSDLHIKEAKID

SEQ ID NO: 23 - protein, 296 aa, Saccharomyces cerevisiae
MKLKREYDELIKADAVKEIAKELGSRPLEVALPEKYIARHEEKFNMACEHILEKDPSLFPILKNNEFTLYLKETQVPNTLEDYFIRLASTILSQQISGQAAESIKARVVSLYGGAFPDYKILFEDFKDPAKCAEIAKCGLSKRKMIYLESLAVYFTEKYKDIEKLFGQKDNDEEVIESLVTNVKGIGPWSAKMFLISGLKRMDVFAPEDLGIARGFSKYLSDKPELEKELMRERKVVKKSKIKHKKYNWKIYDDDIMEKCSETFSPYRSVFMFILWRLASTNTDAMMKAEENFVKS

SEQ ID NO: 24 - protein, 359 aa, Saccharomyces cerevisiae
MWCMRRLPTNSVMTVARKRKQTTIEDFFGTKKSTNEAPNKKGKSGATFMTITNGAAIKTETKAVAKEANTDKYPANSNAKDVYSKNLSSNLRTLLSLELETIDDSWFPHLMDEFKKPYFVKLKQFVTKEQADHTVFPPAKDIYSWTRLTPFNKVKVVIIGQDPYHNFNQAHGLAFSVKPPTPAPPSLKNIYKELKQEYPDFVEDNKVGDLTHWASQGVLLLNTSLTVRAHNANSHSKHGWETFTKRVVQLLIQDREADGKSLVFLLWGNNAIKLVESLLGSTSVGSGSKYPNIMVMKSVHPSPLSASRGFFGTNHFKMINDWLYNTRGEKMIDWSVVPGTSLREVQEANARLESESKDP

SEQ ID NO: 25 - protein, 367 aa, Saccharomyces cerevisiae
MPSTPSFVRSAVSKYKFGAHMSGAGGISNSVTNAFNTGCNSFAMFLKSPRKWVSPQYTQEEIDKFKKNCATYNYNPLTDVLPHGQYFINLANPDREKAEKSYESFMDDLNRCEQLGIGLYNLHPGSTLKGDHQLQLKQLASYLNKAIKETKFVKIVLENMAGTGNLVGSSLVDLKEVIGMIEDKSRIGVCIDTCHTFAAGYDISTTETFNNFWKEFNDVIGFKYLSAVHLNDSKAPLGANRDLHERLGQGYLGIDVFRMIAHSEYLQGIPIVLETPYENDEGYGNEIKLMEWLESKSESELLEDKEYKEKNDTLQKLGAKSRKEQLDKFEVKQKKRAGGTKRKKATAEPSDNDILSQMTKKRKTKKE

SEQ ID NO: 26 - protein, 520 aa, Saccharomyces cerevisiae
MSSSENTLLDGKSENTIRFLTFNVNGIRTFFHYQPFSQMNQSLRSVFDFFRADIITFQELKTEKLSISKWGRVDGFYSFISIPQTRKGYSGVGCWIRIPEKNHPLYHALQVVKAEEGITGYLTIKNGKHSAISYRNDVNQGIGGYDSLDPDLDEKSALELDSEGRCVMVELACGIVIISVYCPANSNSSEEGEMFRLRFLKVLLRRVRNLDKIGKKIVLMGDVNVCRDLIDSADTLEQFSIPITDPMGGTKLEAQYRDKAIQFIINPDTPHRRIFNQILADSLLPDASKRGILIDTTRLIQTRNRLKMYTVWNMLKNLRPSNYGSRIDFILVSLKLERCIKAADILPDILGSDHCPVYSDLDILDDRIEPGTTQVPIPKFEARYKYNLRNHNVLEMFAKKDTNKESNKQKYCVSKVMNTKKNSNIKNKSLDSFFQKVNGEKDDRIKESSEIPQQAKKRISTPKLNFKDVFGKPPLCRHGEESMLKTSKTSANPGRKFWICKRSRGDSNNTESSCGFFQWV

SEQ ID NO: 27 - protein, 238 aa, Saccharomyces cerevisiae
MSHKLTILPFLIKFTPKFPQSIDHDEHGLNVYAFDLDHTIIKPKSPNISFSRSASDWQFINFNSKKSTLDYLCNIIDNDPTAVIVIFSNQGGVITVPRTSKSCTKYTNKILLFLKAIKNDERGETLSHRLWLYAAPKRPKTFAANHSKITFASLGESYNNDPNIFEKVRKPMTGMVEFFKRDLESAYRVSEQISPIKLNWIYYCGDAAGRKKDFSDSDIKFAENLHVEFKYPEEIFHG

SEQ ID NO: 28 - protein, 382 aa, Saccharomyces cerevisiae
MGIKGLNAIISEHVPSAIRKSDIKSFFGRKVAIDASMSLYQFLIAVRQQDGGQLTNEAGETTSHLMGMFYRTLRMIDNGIKPCYVFDGKPPDLKSHELTKRSSRRVETEKKLAEATTELEKMKQERRLVKVSKEHNEEAQKLLGLMGIPYIIAPTEAEAQCAELAKKGKVYAAASEDMDTLCYRTPFLLRHLTFSEAKKEPIHEIDTELVLRGLDLTIEQFVDLCIMLGCDYCESIRGVGPVTALKLIKTHGSIEKIVEFIESGESNNTKWKIPEDWPYKQARMLFLDPEVIDGNEINLKWSPPKEKELIEYLCDDKKFSEERVKSGISRLKKGLKSGIQGRLDGFFQVVPKTKEQLAAAAKRAQENKKLNKNKNKVTKGRR

SEQ ID NO: 29 - protein, 755 aa, Saccharomyces cerevisiae
MRRLLTGCLLSSARPLKSRLPLLMSSSLPSSAGKKPKQATLARFFTSMKNKPTEGTPSPKKSSKHMLEDRMDNVSGEEEYATKKLKQTAVTHTVAAPSSMGSNFSSIPSSAPSSGVADSPQQSQRLVGEVEDALSSNNNDHYSSNIPYSEVCEVFNKIEAISSRLEIIRICSDFFIKIMKQSSKNLIPTTYLFINRLGPDYEAGLELGLGENLLMKTISETCGKSMSQIKLKYKDIGDLGEIAMGARNVQPTMFKPKPLTVGEVFKNLRAIAKTQGKDSQLKKMKLIKRMLTACKGIEAKFLIRSLESKLRIGLAEKTVLISLSKALLLHDENREDSPDKDVPMDVLESAQQKIRDAFCQVPNYEIVINSCLEHGIMNLDKYCTLRPGIPLKPMLAKPTKAINEVLDRFQGETFTSEYKYDGERAQVHLLNDGTMRIYSRNGENMTERYPEINITDFIQDLDTTKNLILDCEAVAWDKDQGKILPFQVLSTRKRKDVELNDVKVKVCLFAFDILCYNDERLINKSLKERREYLTKVTKVVPGEFQYATQITTNNLDELQKFLDESVNHSCEGLMVKMLEGPESHYEPSKRSRNWLKLKKDYLEGVGDSLDLCVLGAYYGRGKRTGTYGGFLLGCYNQDTGEFETCCKIGTGFSDEMLQLLHDRLTPTIIDGPKATFVFDSSAEPDVWFEPTTLFEVLTADLSLSPIYKAGSATFDKGVSLRFPRFLRIREDKGVEDATSSDQIVELYENQSHMQN

SEQ ID NO: 30 - protein, 393 aa, Saccharomyces cerevisiae
MTDIQLNGKSTLDTPSATMSAKEKEAKLKSADENNKPPNYKRISDDDLYRHSSQYRMWSYTKDQLQEKRVDTNARAIAYIEENLLKFREAHNLTEEEIKVLEAKAIPLTMEEELDLVNFYAKKVQVIAQHLNLPTEVVATAISFFRRFFLENSVMQIDPKSIVHTTIFLACKSENYFISVDSFAQKAKSTRDSVLKFEFKLLESLKFSLLNHHPYKPLHGFFLDIQNVLYGKVDLNYMGQIYDRCKKRITAALLTDVVYFYTPPQITLATLLIEDEALVTRYLETKFPSREGSQESVPGNEKEEPQNDASTTEKNKEKSTESEEYSIDSAKLLTIIRECKSIIEDCKPPSTEEAKKIAAKNYYCQNPSTLIQKLKRKLNGEDTSSTVEKKQKT

SEQ ID NO: 31 - protein, 306 aa, Saccharomyces cerevisiae
MKVNMEYTKEKKVGEGTYAVVYLGCQHSTGRKIAIKEIKTSEFKDGLDMSAIREVKYLQEMQHPNVIELIDIFMAYDNLNLVLEFLPTDLEVVIKDKSILFTPADIKAWMLMTLRGVYHCHRNFILHRDLKPNNLLFSPDGQIKVADFGLARAIPAPHEILTSNVVTRWYRAPELLFGAKHYTSAIDIWSVGVIFAELMLRIPYLPGQNDVDQMEVTFRALGTPTDRDWPEVSSFMTYNKLQIYPPPSRDELRKRFIAASEYALDFMCGMLTMNPQKRWTAVQCLESDYFKELPPPSDPSSIKIRN

SEQ ID NO: 32 - protein, 210 aa, Saccharomyces cerevisiae
MNNTDPTSFESILAGVAKLRKEKSGADTTGSQSLEIDASKLQQQEPQTSRRINSNQVINAFNQQKPEEWTDSKATDDYNRKRPFRSTRPGKTVLVNTTQKENPLLNHLKSTNWRYVSSTGINMIYYDYLVRGRSVLFLTLTYHKLYVDYISRRMQPLSRNENNILIFIVDDNNSEDTLNDITKLCMFNGFTLLLAFNFEQAAKYIEYLNL

SEQ ID NO: 33 - protein, 1031 aa, Saccharomyces cerevisiae
MGVHSFWDIAGPTARPVRLESLEDKRMAVDASIWIYQFLKAVRDQEGNAVKNSHITGFFRRICKLLYFGIRPVFVFDGGVPVLKRETIRQRKERRQGKRESAKSTARKLLALQLQNGSNDNVKNSTPSSGSSVQIFKPQDEWDLPDIPGFKYDKEDARVNSNKTFEKLMNSINGDGLEDIDLDTINPASAEFEELPKATQYLILSSLRLKSRLRMGYSKEQLETIFPNSMDFSRFQIDMVKRRNFFTQKLINTTGFQDGGASKLNEEVINRISGQKSKEYKLTKTNNGWILGLGANDGSDAQKAIVIDDKDAGALVKQLDSNAEDGDVLRWDDLEDNSLKIVRHESSNATTAPQKRSNRSEDEGCDSDECEWEEVELKPKNVKFVEDFSLKAARLPYMGQSLNNAGSKSFLDKRHDQASPSKTTPTMRISRISVEDDDEDYLKQIEEIEMMEAVQLSKMEKKPEADDKSKIAKPVTSKGTEARPPIVQYGLLGAQPDSKQPYHVTNLNSKSESVIKRTSKTVLSEFRPPSQQEDKGAILTEGEQNLNFISHKIPQFDFNNENSLLFQKNTESNVSQEATKEKSPIPEMPSWFSSTASQQLYNPYNTTNFVEDKNVRNEQESGAETTNKGSSYELLTGLNATEILERESEKESSNDENKDDDLEVLSEELFEDVPTKSQISKEAEDNDSRKVESINKEHRKPLIFDYDFSEDEEDNIVENMIKEQEEFDTFKNTTLSTSAERNVAENAFVEDELFEQQMKDKRDSDEVTMDMIKEVQELLSRFGIPYITAPMEAEAQCAELLQLNLVDGIITDDSDVFLFGGTKIYKNMFHEKNYVEFYDAESILKLLGLDRKNMIELAQLLGSDYTNGLKGMGPVSSIEVIAEFGNLKNFKDWYNNGQFDKRKQETENKFEKDLRKKLVNNEIILDDDFPSVMVYDAYMRPEVDHDTTPFVWGVPDLDMLRSFMKTQLGWPHEKSDEILIPLIRDVNKRKKKGKQKRINEFFPREYISGDKKLNTSKRISTATGKLKKRKM

SEQ ID NO: 34 - protein, 843 aa, Saccharomyces cerevisiae
MTDVEGYQPKSKGKIFPDMGESFFSSDEDSPATDAEIDENYDDNRETSEGRGERDTGAMVTGLKKPRKKTKSSRHTAADSSMNQMDAKDKALLQDTNSDIPADFVPDSVSGMFRSHDFSYLRLRPDHASRPLWISPSDGRIILESFSPLAEQAQDFLVTIAEPISRPSHIHEYKITAYSLYAAVSVGLETDDIISVLDRLSKVPVAESIINFIKGATISYGKVKLVIKHNRYFVETTQADILQMLLNDSVIGPLRIDSDHQVQPPEDVLQQQLQQTAGKPATNVNPNDVEAVFSAVIGGDNEREEEDDDIDAVHSFEIANESVEVVKKRCQEIDYPVLEEYDFRNDHRNPDLDIDLKPSTQIRPYQEKSLSKMFGNGRARSGIIVLPCGAGKTLVGITAACTIKKSVIVLCTSSVSVMQWRQQFLQWCTLQPENCAVFTSDNKEMFQTESGLVVSTYSMVANTRNRSHDSQKVMDFLTGREWGFIILDEVHVVPAAMFRRVVSTIAAHAKLGLTATLVREDDKIGDLNFLIGPKLYEANWMELSQKGHIANVQCAEVWCPMTAEFYQEYLRETARKRMLLYIMNPTKFQACQFLIQYHERRGDKIIVFSDNVYALQEYALKMGKPFIYGSTPQQERMNILQNFQYNDQINTIFLSKVGDTSIDLPEATCLIQISSHYGSRRQEAQRLGRILRAKRRNDEGFNAFFYSLVSKDTQEMYYSTKRQAFLVDQGYAFKVITHLHGMENIPNLAYASPRERRELLQEVLLKNEEAAGIEVGDDADNSVGRGSNGHKRFKSKAVRGEGSLSGLAGGEDMAYMEYSTNKNKELKEHHPLIRKMYYKNLKK

SEQ ID NO: 35 - protein, 1100 aa, Saccharomyces cerevisiae
MSQLFYQGDSDDELQEELTRQTTQASQSSKIKNEDEPDDSNHLNEVENEDSKVLDDDAVLYPLIPNEPDDIETSKPNINDIRPVDIQLTLPLPFQQKVVENSLITEDALIIMGKGLGLLDIVANLLHVLATPTSINGQLKRALVLVLNAKPIDNVRIKEALEELSWFSNTGKDDDDTAVESDDELFERPFNVVTADSLSIEKRRKLYISGGILSITSRILIVDLLSGIVHPNRVTGMLVLNADSLRHNSNESFILEIYRSKNTWGFIKAFSEAPETFVMEFSPLRTKMKELRLKNVLLWPRFRVEVSSCLNATNKTSHNKVIEVKVSLTNSMSQIQFGLMECLKKCIAELSRKNPELALDWWNMENVLDINFIRSIDSVMVPNWHRISYESKQLVKDIRFLRHLLKMLVTSDAVDFFGEIQLSLDANKPSVSRKYSESPWLLVDEAQLVISYAKKRIFYKNEYTLEENPKWEQLIHILHDISHERMTNHLQGPTLVACSDNLTCLELAKVLNASNKKRGVRQVLLNKLKWYRKQREETKKLVKEVQSQDTFPENATLNVSSTFSKEQVTTKRRRTRGASQVAAVEKLRNAGTNVDMEVVFEDHKLSEEIKKGSGDDLDDGQEENAANDSKIFEIQEQENEILIDDGDAEFDNGELEYVGDLPQHITTHFNKDLWAEHCNEYEYVDRQDEILISTFKSLNDNCSLQEMMPSYIIMFEPDISFIRQIEVYKAIVKDLQPKVYFMYYGESIEEQSHLTAIKREKDAFTKLIRENANLSHHFETNEDLSHYKNLAERKLKLSKLRKSNTRNAGGQQGFHNLTQDVVIVDTREFNASLPGLLYRYGIRVIPCMLTVGDYVITPDICLERKSISDLIGSLQNNRLANQCKKMLKYYAYPTLLIEFDEGQSFSLEPFSERRNYKNKDISTVHPISSKLSQDEIQLKLAKLVLRFPTLKIIWSSSPLQTVNIILELKLGREQPDPSNAVILGTNKVRSDFNSTAKGLKDGDNESKFKRLLNVPGVSKIDYFNLRKKIKSFNKLQKLSWNEINELINDEDLTDRIYYFLRTEKEEQEQESTDENLESPGKTTDDNALHDHHNDVPEAPV

SEQ ID NO: 36 - protein, 1085 aa, Saccharomyces cerevisiae
MEDKEQQDNAKLENNESLKDLGVNVLSQSSLEEKIANDVTNFSNLQSLQQEETRLERSKTALQRYVNKKNHLTRKLNNTTRISVKQNLRDQIKNLQSDDIERVLKDIDDIQSRIKELKEQVDQGAENKGSKEGLQRPGETEKEFLIRTGKITAFGHKAGFSLDTANREYAKNDEQKDEDFEMATEQMVENLTDEDDNLSDQDYQMSGKESEDDEEEENDDKILKELEDLRFRGQPGEAKDDGDELYYQERLKKWVKQRSCGSQRSSDLPEWRRPHPNIPDAKLNSQFKIPGEIYSLLFNYQKTCVQWLYELYQQNCGGIIGDEMGLGKTIQVIAFIAALHHSGLLTGPVLIVCPATVMKQWCNEFQHWWPPLRTVILHSMGSGMASDQKFKMDENDLENLIMNSKPSDFSYEDWKNSTRTKKALESSYHLDKLIDKVVTDGHILITTYVGLRIHSDKLLKVKWQYAVLDEGHKIRNPDSEISLTCKKLKTHNRIILSGTPIQNNLTELWSLFDFIFPGKLGTLPVFQQQFVIPINIGGYANATNIQVQTGYKCAVALRDLISPYLLRRVKADVAKDLPQKKEMVLFCKLTKYQRSKYLEFLHSSDLNQIQNGKRNVLFGIDILRKICNHPDLLDRDTKRHNPDYGDPKRSGKMQVVKQLLLLWHKQGYKALLFTQSRQMLDILEEFISTKDPDLSHLNYLRMDGTTNIKGRQSLVDRFNNESFDVFLLTTRVGGLGVNLTGANRIIIFDPDWNPSTDMQARERAWRIGQKREVSIYRLMVGGSIEEKIYHRQIFKQFLTNRILTDPKQKRFFKIHELHDLFSLGGENGYSTEELNEEVQKHTENLKNSKSEESDDFEQLVNLSGVSKLESFYNGKEKKENSKTEDDRLIEGLLGGESNLETVMSHDSVVNSHAGSSSSNIITKEASRVAIEAVNALRKSRKKITKQYEIGTPTWTGRFGKAGKIRKRDPLKNKLTGSAAILGNITKSQKEASKEARQENYDDGITFARSKEINSNTKTLENIRAYLQKQNNFFSSSVSILNSIGVSLSDKEDVIKVRALLKTIAQFDKERKGWVLDEEFRNNNAS

SEQ ID NO: 37 - protein, 506 aa, Saccharomyces cerevisiae
MDPFLEFRVGNISLNEFYRRTIQSEFERILEDPLSNMKNYRFSKQSNYSTKEKTPLSIGVNCLDIDDTGQVLLGGGDDGSLSIWGLDESLHRNDEGEQELINKRLNYIKRQPHQSDDEPAQIMGYKNKRTRINDNNTMRLVHSFQTQRNKYRMYRQSSAAVPVQRSHISNKTDSPIGFSETLSETDSEASISHHKYGITTLKWYKADNGMFFTGSNDKTVKIWDTNRFEAVQDINLGYKINQIDNNVVDDSSLLVVASEDYYPRLIDLRTMNSGVTALGMGNQTRMQSEILCCKFNPVREQIIACGDMEGGVKLWDLRMRNRLYSELKRNKNRFKTINNDDNDDQSDVYFSSNQSKAHLRCCSDIVWNSEGSELCSVGMDGKLNVWRPFTEILQPEGLASYSQLGTQDLSRIKYKKRVSRRLLWFDKFLLCITDNGEVEIYNTEEKKLWNKLEYPMVNQVKKNQASHCQFSSMIVQTNIMNSVGLKLFFGTNNNTVSDGGSIFECS

SEQ ID NO: 38 - protein, 321 aa, Saccharomyces cerevisiae
MLMDEYEENKDMCPICKTDRYLSPDVKFLVNPECYHRICESCVDRIFSLGPAQCPYKGCDKILRKNKFKTQIFDDVEVEKEVDIRKRVFNVFNKTIDDFNGDLVEYNKYLEEVEDIIYKLDHGIDVAKTEEKLRTYEELNKQLIMNNLERSRTEIESFEQRQKFEKEMKLKKRLLERQIEEEERMNKEWTKKEIVNRLSTTTQDINETIEGVKNTVKLKKSSARRKLEELNRVLKNNPYFNSNVNVQNSRLKDAVPFTPFNGDREAHPRFTLKGSVYNDPFIKDLEHRKEFIASGFNTNYAYERVLTEAFMGLGCVISEEL

SEQ ID NO: 39 - protein, 1032 aa, Saccharomyces cerevisiae
MTPDELNSAVVTFMANLNIDDSKANETASTVTDSIVHRSIKLLEVVVALKDYFLSENEVERKKALTCLTTILAKTPKDHLSKNECSVIFQFYQSKLDDQALAKEVLEGFAALAPMKYVSINEIAQLLRLLLDNYQQGQHLASTRLWPFKILRKIFDRFFVNGSSTEQVKRINDLFIETFLHVANGEKDPRNLLLSFALNKSITSSLQNVENFKEDLFDVLFCYFPITFKPPKHDPYKISNQDLKTALRSAITATPLFAEDAYSNLLDKLTASSPVVKNDTLLTLLECVRKFGGSSILENWTLLWNALKFEIMQNSEGNENTLLNPYNKDQQSDDVGQYTNYDACLKIINLMALQLYNFDKVSFEKFFTHVLDELKPNFKYEKDLKQTCQILSAIGSGNVEIFNKVISSTFPLFLINTSEVAKLKLLIMNFSFFVDSYIDLFGRTSKESLGTPVPNNKMAEYKDEIIMILSMALTRSSKAEVTIRTLSVIQFTKMIKMKGFLTPEEVSLIIQYFTEEILTDNNKNIYYACLEGLKTISEIYEDLVFEISLKKLLDLLPDCFEEKIRVNDEENIHIETILKIILDFTTSRHILVKESITFLATKLNRVAKISKSREYCFLLISTIYSLFNNNNQNENVLNEEDALALKNAIEPKLFEIITQESAIVSDNYNLTLLSNVLFFTNLKIPQAAHQEELDRYNELFISEGKIRILDTPNVLAISYAKILSALNKNCQFPQKFTVLFGTVQLLKKHAPRMTETEKLGYLELLLVLSNKFVSEKDVIGLFDWKDLSVINLEVMVWLTKGLIMQNSLESSEIAKKFIDLLSNEEIGSLVSKLFEVFVMDISSLKKFKGISWNNNVKILYKQKFFGDIFQTLVSNYKNTVDMTIKCNYLTALSLVLKHTPSQSVGPFINDLFPLLLQALDMPDPEVRVSALETLKDTTDKHHTLITEHVSTIVPLLLSLSLPHKYNSVSVRLIALQLLEMITTVVPLNYCLSYQDDVLSALIPVLSDKKRIIRKQCVDTRQVYYELGQIPFE

SEQ ID NO: 40 - protein, 621 aa, Saccharomyces cerevisiae
MSSVQLSRGDFHSIFTNKQRYDNPTGGVYQVYNTRKSDGANSNRKNLIMISDGIYHMKALLRNQAASKFQSMELQRGDIIRVIIAEPAIVRERKKYVLLVDDFELVQSRADMVNQTSTFLDNYFSEHPNETLKDEDITDSGNVANQTNASNAGVPDMLHSNSNLNANERKFANENPNSQKTRPIFAIEQLSPYQNVWTIKARVSYKGEIKTWHNQRGDGKLFNVNFLDTSGEIRATAFNDFATKFNEILQEGKVYYVSKAKLQPAKPQFTNLTHPYELNLDRDTVIEECFDESNVPKTHFNFIKLDAIQNQEVNSNVDVLGIIQTINPHFELTSRAGKKFDRRDITIVDDSGFSISVGLWNQQALDFNLPEGSVAAIKGVRVTDFGGKSLSMGFSSTLIPNPEIPEAYALKGWYDSKGRNANFITLKQEPGMGGQSAASLTKFIAQRITIARAQAENLGRSEKGDFFSVKAAISFLKVDNFAYPACSNENCNKKVLEQPDGTWRCEKCDTNNARPNWRYILTISIIDETNQLWLTLFDDQAKQLLGVDANTLMSLKEEDPNEFTKITQSIQMNEYDFRIRAREDTYNDQSRIRYTVANLHSLNYRAEADYLADELSKALLA

SEQ ID NO: 41 - protein, 273 aa, Saccharomyces cerevisiae
MATYQPYNEYSSVTGGGFENSESRPGSGESETNTRVNTLTPVTIKQILESKQDIQDGPFVSHNQELHHVCFVGVVRNITDHTANIFLTIEDGTGQIEVRKWSEDANDLAAGNDDSSGKGYGSQVAQQFEIGGYVKVFGALKEFGGKKNIQYAVIKPIDSFNEVLTHHLEVIKCHSIASGMMKQPLESASNNNGQSLFVKDDNDTSSGSSPLQRILEFCKKQCEGKDANSFAVPIPLISQSLNLDETTVRNCCTTLTDQGFIYPTFDDNNFFAL

SEQ ID NO: 42 - protein, 859 aa, Saccharomyces cerevisiae
MSAYIAMKGVITNVDENIRNDEDVAFEYEIQKTPQNILTWKRYIEYWKEEGRTDKQIRWLYERFCSQFVTDTSIWEDYIRWESTKEVVETSRIFWLFQRCLKSCVRDCDRICLSYLELAIEQYDLAMIRHALASSLMKMEREMHRKVWDPVIKFVEEKVLPLTQLDSTQEDEEESTDEAELINVLLVKGFTKGGFISEEISENGSRGDIWSSHILERYLKVAPQQKRNESLATLALTRDNITIKSVYEKYLPQDENSGKYLPSSELPFELNFNYLASLEKLGLDNQYEEFMRQMNGIYPDKWLFLILSLAKYYISRGRLDSCGDLLKKSLQQTLRYSDFDRIYNFYLLFEQECSQFILGKLKENDSKFFNQKDWTEKLQAHMATFESLINLYDIYLNDVALRQDSNLVETWMKRVSLQKSAAEKCNVYSEAILKIDPRKVGTPGSFGRLWCSYGDLYWRSNAISTARELWTQSLKVPYPYIEDLEEIYLNWADRELDKEGVERAFSILEDALHVPTNPEILLEKYKNGHRKIPAQTVLFNSLRIWSKYIDYLEAYCPKDANSSDKIFNKTKMAYNTVIDLRLITPAMAENFALFLQNHYEVMESFQVYEKTIPLFPPEIQYELWIEYLEVATSHQLSSLSPEHIRFLFEKALKNLCSNGIDCKTIFIAYSVFEERISGLISKSIEILRRGAVIGTVSVSTHLESRLQLWRMCISKAESTLGPSVTRELYQECIQILPNSKAVEFVIKFSDFESSIGETIRAREILAYGAKLLPPSRNTELWDSFEIFELKHGDKETYKDMLKMKKVLESNMLIDSASVSHEEGNINFVAAATSHAPNSHTLTQSTSSYSINPDEIELDI

SEQ ID NO: 43 - protein, 371 aa, Saccharomyces cerevisiae
MTPEQKAKLEANRKLAIERLRKRGILSSDQLNRIESRNEPLKTRPLAVTSGSNRDDNAAAAVHVPNHNGQPSALANTNTNTTSLYGSGVVDGSKRDASVLDKRPTDRIRPSIRKQDYIEYDFATMQNLNGGYINPKDKLPNSDFTDDQEFESEFGSKKQKTLQDWKKEQLERKMLYENAPPPEHISKAPKCIECHINIEMDPVLHDVFKLQVCKQCSKEHPEKYALLTKTECKEDYFLTDPELNDEDLFHRLEKPNPHSGTFARMQLFVRCEVEAFAFKKWGGEEGLDEEWQRREEGKAHRREKKYEKKIKEMRLKTRAQEYTNRLREKKHGKAHIHHFSDPVDGGIDEDGYQIQRRRCTDCGLETEEIDI

SEQ ID NO: 44 - protein, 754 aa, Saccharomyces cerevisiae
MNEDLPKEYFELIRKALNEKEAEKAPLSRRRRVRRKNQPLPDAKKKFKTGLNELPRESVVTVNLDSSDDGVVTVPTDDSVEEIQSSEEDYDSEEFEDVTDGNEVAGVEDISVEIKPSSKRNSDARRTSRNVCSNEERKRRKYFHMLYLVCLMVHGFIRNEWINSKRLSRKLSNLVPEKVFELLHPQKDEELPLRSTRKLLDGLKKCMELWQKHWKITKKYDNVGLYMRTWKEIEMSANNKRKFKTLKRSDFLRAVSKGHGDPDISVQGFVAMLRACNVNARLIMSCQPPDFTNMKIDTSLNGNNAYKDMVKYPIFWCEVWDKFSKKWITVDPVNLKTIEQVRLHSKLAPKGVACCERNMLRYVIAYDRKYGCRDVTRRYAQWMNSKVRKRRITKDDFGEKWFRKVITALHHRKRTKIDDYEDQYFFQRDESEGIPDSVQDLKNHPYYVLEQDIKQTQIVKPGCKECGYLKVHGKVGKVLKVYAKRDIADLKSARQWYMNGRILKTGSRCKKVIKRTVGRPKGEAEEEDERLYSFEDTELYIPPLASASGEITKNTFGNIEVFAPTMIPGNCCLVENPVAIKAARFLGVEFAPAVTSFKFERGSTVKPVLSGIVVAKWLREAIETAIDGIEFIQEDDNRKEHLLGALESWNTLLLKLRIRSKLNSTYGKIAEEEPNVTKEQNIADNHDNTETFMGGGFLPGIANHEARPYSEPSEPEDSLDYVSVDKAEESATDDDVGEDYSDFMKELEMSEESD

SEQ ID NO: 45 - protein, 964 aa, Saccharomyces cerevisiae [sequence omitted]

SEQ ID NO: 46 - protein, 1242 aa, Saccharomyces cerevisiae
MAPATPKTSKTAHFENGSTSSQKKMKQSSLLSFFSKQVPSGTPSKKVQKPTPATLENTATDKITKNPQGGKTGKLFVDVDEDNDLTIAEETVSTVRSDIMHSQEPQSDTMLNSNTTEPKSTTTDEDLSSSQSRRNHKRRVNYAESDDDDSDTTFTAKRKKGKVVDSESDEDEYLPDKNDGDEDDDIADDKEDIKGELAEDSGDDDDLISLAETTSKKKFSYNTSHSSSPFTRNISRDNSKKKSRPNQAPSRSYNPSHSQPSATSKSSKFNKQNEERYQWLVDERDAQRRPKSDPEYDPRTLYIPSSAWNKFTPFEKQYWEIKSKMWDCIVFFKKGKFFELYEKDALLANALFDLKIAGGGRANMQLAGIPEMSFEYWAAQFIQMGYKVAKVDQRESMLAKEMREGSKGIVKRELQCILTSGTLTDGDMLHSDLATFCLAIREEPGNFYNETQLDSSTIVQKLNTKIFGAAFIDTATGELQMLEFEDDSECTKLDTLMSQVRPMEVVMERNNLSTLANKIVKFNSAPNAIFNEVKAGEEFYDCDKTYAEIISSEYFSTEEDWPEVLKSYYDTGKKVGFSAFGGLLYYLKWLKLDKNLISMKNIKEYDFVKSQHSMVLDGITLQNLEIFSNSFDGSDKGTLFKLFNRAITPMGKRMMKKWLMHPLLRKNDIESRLDSVDSLLQDITLREQLEITFSKLPDLERMLARIHSRTIKVKDFEKVITAFETIIELQDSLKNNDLKGDVSKYISSFPEGLVEAVKSWTNAFERQKAINENIIVPQRGFDIEFDKSMDRIQELEDELMEILMTYRKQFKCSNIQYKDSGKEIYTIEIPISATKNVPSNWVQMAANKTYKRYYSDEVRALARSMAEAKEIHKTLEEDLKNRLCQKFDAHYNTIWMPTIQAISNIDCLLAITRTSEYLGAPSCRPTIVDEVDSKTNTQLNGFLKFKSLRHPCFNLGATTAKDFIPNDIELGKEQPRLGLLTGANAAGKSTILRMACIAVIMAQMGCYVPCESAVLTPIDRIMTRLGANDNIMQGKSTFFVELAETKKILDMATNRSLLVVDELGRGGSSSDGFAIAESVLHHVATHIQSLGFFATHYGTLASSFKHHPQVRPLKMSILVDEATRNVTFLYKMLEGQSEGSFGMHVASMCGISKEIIDNAQIAADNLEHTSRLVKERDLAANNLNGEVVSVPGGLQSDFVRIAYGDGLKNTKLGSGEGVLNYDWNIKRNVLKSLFSIIDDLQS

SEQ ID NO: 47 - protein, 1018 aa, Saccharomyces cerevisiae
MAGQPTISRFFKKAVKSELTHKQEQEVAVGNGAGSESICLDTDEEDNLSSVASTTVTNDSFPLKGSVSSKNSKNSEKTSGTSTTFNDIDFAKKLDRIMKRRSDENVEAEDDEEEGEEDFVKKKARKSPTAKLTPLDKQVKDLKMHHRDKVLVIRVGYKYKCFAEDAVTVSRILHIKLVPGKLTIDESNPQDCNHRQFAYCSFPDVRLNVHLERLVHHNLKVAVVEQAETSAIKKHDPGASKSSVFERKISNVFTKATFGVNSTFVLRGKRILGDTNSIWALSRDVHQGKVAKYSLISVNLNNGEVVYDEFEEPNLADEKLQIRIKYLQPIEVLVNTDDLPLHVAKFFKDISCPLIHKQEYDLEDHVVQAIKVMNEKIQLSPSLIRLVSKLYSHMVEYNNEQVMLIPSIYSPFASKIHMLLDPNSLQSLDIFTHDGGKGSLFWLLDHTRTSFGLRMLREWILKPLVDVHQIEERLDAIECITSEINNSIFFESLNQMLNHTPDLLRTLNRIMYGTTSRKEVYFYLKQITSFVDHFKMHQSYLSEHFKSSDGRIGKQSPLLFRLFSELNELLSTTQLPHFLTMINVSAVMEKNSDKQVMDFFNLNNYDCSEGIIKIQRESESVRSQLKEELAEIRKYLKRPYLNFRDEVDYLIEVKNSQIKDLPDDWIKVNNTKMVSRFTTPRTQKLTQKLEYYKDLLIRESELQYKEFLNKITAEYTELRKITLNLAQYDCILSLAATSCNVNYVRPTFVNGQQAIIAKNARNPIIESLDVHYVPNDIMMSPENGKINIITGPNMGGKSSYIRQVALLTIMAQIGSFVPAEEIRLSIFENVLTRIGAHDDIINGDSTFKVEMLDILHILKNCNKRSLLLLDEVGRGTGTHDGIAISYALIKYFSELSDCPLILFTTHFPMLGEIKSPLIRNYHMDYVEEQKTGEDWMSVIFLYKLKKGLTYNSYGMNVAKLARLDKDIINRAFSISEELRKESINEDALKLFSSLKRILKSDNITATDKLAKLLSLDIH

SEQ ID NO: 48 - protein, 769 aa, Saccharomyces cerevisiae
MSLRIKALDASVVNKIAAGEIIISPVNALKEMMENSIDANATMIDILVKEGGIKVLQITDNGSGINKADLPILCERFTTSKLQKFEDLSQIQTYGFRGEALASISHVARVTVTTKVKEDRCAWRVSYAEGKMLESPKPVAGKDGTTILVEDLFFNIPSRLRALRSHNDEYSKILDVVGRYAIHSKDIGFSCKKFGDSNYSLSVKPSYTVQDRIRTVFNKSVASNLITFHISKVEDLNLESVDGKVCNLNFISKKSISPIFFINNRLVTCDLLRRALNSVYSNYLPKGNRPFIYLGIVIDPAAVDVNVHPTKREVRFLSQDEIIEKIANQLHAELSAIDTSRTFKASSISTNKPESLIPFNDTIESDRNRKSLRQAQVVENSYTTANSQLRKAKRQENKLVRIDASQAKITSFLSSSQQFNFEGSSTKRQLSEPKVTNVSHSQEAEKLTLNESEQPRDANTINDNDLKDQPKKKQKLGDYKVPSIADDEKNALPISKDGYIRVPKERVNVNLTSIKKLREKVDDSIHRELTDIFANLNYVGVVDEERRLAAIQHDLKLFLIDYGSVCYELFYQIGLTDFANFGKINLQSTNVSDDIVLYNLLSEFDELNDDASKEKIISKIWDMSSMLNEYYSIELVNDGLDNDLKSVKLKSLPLLLKGYIPSLVKLPFFIYRLGKEVDWEDEQECLDGILREIALLYIPDMVPKVDTSDASLSEDEKAQFINRKEHISSLLEHVLFPCIKRRFLAPRHILKDVVEIANLPDLYKVFERC

SEQ ID NO: 49 - protein, 873 aa, Saccharomyces cerevisiae
MTQIHQINDIDVHRITSGQVITDLTTAVKELVDNSIDANANQIEIIFKDYGLESIECSDNGDGIDPSNYEFLALKHYTSKIAKFQDVAKVQTLGFRGEALSSLCGIAKLSVITTTSPPKADKLEYDMVGHITSKTTTSRNKGTTVLVSQLFHNLPVRQKEFSKTFKRQFTKCLTVIQGYAIINAAIKFSVWNITPKGKKNLILSTMRNSSMRKNISSVFGAGGMRGLEEVDLVLDLNPFKNRMLGKYTDDPDFLDLDYKIRVKGYISQNSFGCGRNSKDRQFIYVNKRPVEYSTLLKCCNEVYKTFNNVQFPAVFLNLELPMSLIDVNVTPDKRVILLHNERAVIDIFKTTLSDYYNRQELALPKRMCSQSEQQAQKRLKTEVFDDRSTTHESDNENYHTARSESNQSNHAHFNSTTGVIDKSNGTELTSVMDGNYTNVTDVIGSECEVSVDSSVVLDEGNSSTPTKKLPSIKTDSQNLSDLNLNNFSNPEFQNITSPDKARSLEKVVEEPVYFDIDGEKFQEKAVLSQADGLVFVDNECHEHTNDCCHQERRGSTDTEQDDEADSIYAEIEPVEINVRTPLKNSRKSISKDNYRSLSDGLTHRKFEDEILEYNLSTKNFKEISKNGKQMSSIISKRKSEAQENIIKNKDELEDFEQGEKYLTLTVSKNDFKKMEVVGQFNLGFIIVTRKVDNKYDLFIVDQHASDEKYNFETLQAVTVFKSQKLIIPQPVELSVIDELVVLDNLPVFEKNGFKLKIDEEEEFGSRVKLLSLPTSKQTLFDLGDFNELIHLIKEDGGLRRDNIRCSKIRSMFAMRACRSSIMIGKPLNKKTMTRVVHNLSELDKPWNCPHGRPTMRHLMELRDWSSFSKDYEI

SEQ ID NO: 50 - protein, 848 aa, Candida albicans
MAVRKPLTQFTTPLGSGAGVPKDKRPPRVTDSATRLSKPFKVPFSSSTQNSVSSETITISRRPVRSTRKRTNYAVDGITSEKDDEEEYNNDQNVNGDGQIIKRKKFGALLPRSIINSDNIAKNEDKFKRSFTVPFDKNNSKQTEIQRGPPPPLGTKVRAILPPRALHDPTSEFAIVLYDPTVDKIPKISEVESSTSSSTSPEPEPETKETEERPIKRKRTHKSLAEILGIVTNPEEKLSKYPDVPVVIDPKLAKILRPHQIAGVKFLYRCTAGLIDARAKGCIMADEMGLGKTLQCLTLMWTLLRQSPRGKRTIEKCIIVCPSSLVRNWANEIVKWLGEGALTPLAVDGKSTKNSELGTALQQWSTAQGRNIVRPVLIISYETLRRNVDKLAGTEVGLMLADEGHRLKNGDSLTFTALNSLRCERRVILSGTPIQNDLSEYFSLLNFANPGYLGTRIEFKKNYENAILKGRDSTASDEEREKGDKKLNELSQMVSKFIIRRTNDILSKYLPIKYEYVLFTGLSPMQKDIYNHFITSPEIKKLMKGTGSQPLKAIGMLKKLCNHPDLLDLPEDVEGSEEFIPDDYQSSIAGGSASRNREIQTWFSGKFLILERFLQKINKETDDKIVLISNYTQTLDLIEKMCRYKKYGVLRLDGTMNINKRQKLVDKFNDPNGPEFIFLLSSKAGGCGINLIGANRLVLMDPDWNPASDQQALARVWRDGQKKDCFIYRFISTGTIEEKIFQRQSMKMSLSSCVVDEKEDVERLFSVANLRQLFKFQPDTQCDTHDTFNCNRCKGDKGQFIKAPAMLYGDATTWNHLNHQALANNEDILITNEAQHNDVTFAFQYISH

SEQ ID NO: 51 - protein, 361 aa, Candida albicans
MTQTEIEQVDLHEEGSHPQNINADAEVEAEEDEDVLNGPLLIEQLEGNGINASDIKKLKAEGFHTIESIAYTPKRHLMTVKGISENKAEKISAEAAKLVPLGFTTASEFHSRRSELICLTTGSKQLDTLLGGGVETGSITEVFGEFRTGKSQLCHTLAVTCQLPIDMGGGEGKCLYIDTEGTFRPNRLVSIAQRYGLNPNDCLDNVAYARAYNAEHQLNLLNIAAEMMAESRFSLLIVDSIMSLYRTDYAGRGELSARQTHVAKFMRTLQRLADEFGIAVVITNQVVAQVDGMSGMFNPDPKKPIGGNIIAHSSTTRLSFKKGRGETRICKIYDSPCLPESECIFAIYEDGIGDPKVEDNE

SEQ ID NO: 52 - protein, 564 aa, Candida albicans
MNSRPAPPQPRPPQQQPQQPQQPQPNQQRLPFRPDESRARRLNQPLEQHANPINPSNFAAREYTEQEHKDIQTKLNKSLGPEFISYRDGPGGTRVQYIEGWKILNLANEIFGFNGWNSQIVSCQVDFLDTHGGSGKFSVGISMVVRITIRDGTFHEDVGYGSIENCRSKILALERCKKEALTDGLKRCLRCFGNVLGNCLYDKTIVAQMKKLKKFPIEFDPNDYYRDPLLVERERKKNIIEKQHEEQKRQQEDASRLDNSGQQVQHQQQNNSNVVSNGSIPVNGQQNHQNKGSDIVEPLEANQGNTRAGQVPQLSHHAAAFVTPKSPTKVVSYVPTSKEAEELDDSFMFSDDIFDGESQSIYYPPEVREHEQNQQQQKEENQGVARNGATKETTNNGRSENRPQNESMAVDQNPPVLMNAFVTAKSAEVLQQSPTVEAAAKNIVQFDPKFVSPNMRRTVDPTKSVPIKRSDIKNSVATTPSPLGVNTSSNRINKPMITSNNNNNINNMGKRIGMPPQPRTNKLVNSGNNPSSGIPHTVSTSVNGASGNENVENNSTIANTTVNQ

SEQ ID NO: 53 - protein, 406 aa, Saccharomyces cerevisiae
MSLGIPLSQLIVESPKPLSSGITGLDEILNLGFQARSIYEIFGPPGIGKTNFGIQLVCNSLEGIQQSEINDDKILWIETFQEMPINILRERFQKFKIVEENVKRVRITKFGQLLYFFQNLFKLSQSVRYKLVIIDGFSQLVCDHLCTLSKRGGGMIDKTIHELKCRHLILIFTVMTKYTHSTGSTIIVLNDCMNTAFQSNEFESLEEYYEILDDGSNFFVNSNNERRKNNVHILKSALVANIAMGSKDSTWEVFLRDRIGLFRDWNEQVDETVFVKSKRVKASSSQSNEGCTTIKEMRINKRNFENLRIAIVFNLHGEDRKREGRNLKRSRSSDDRNYIVKFDFDKATGQLRDIIDLKPDTANIASFPTLSTSSSSCSQVFNNIDSNDNPLPNAEGKEEIIYDSEG

SEQ ID NO: 54 - protein, 511 aa, Candida albicans
MDDYKQLGPGIFSCSRKFPFLTTRLQQYGKSINDLLQYDEASDHAQLARLVDRPMKEINDYYRNLKKDLQVDPTSVNSLLDSEFISTGLPSIDRELGGGIPIGEVTEIFGASGCGKSHFLFQLLSNCGKEFSTSKNIYISTESFLETKRLKDFIGRNSSNIDTDLDRISYIYCQDLESQDHILFTQLPLKLDSDKGKTKLLVLDSIAQHFRREDSIMNSTYLKEKLEVQEGEIADDRSFQEVKRKQMNQLRRFNKSQKYASRTAKLYYICQLYQHLSRLAQDFNIAIVIVNQVSDYSFESSGSFTEAEAGELDYPLNLDFQTAVSSGWDSRTIYNSIPTANITLKNEEIENLEMELAKSLEERSPNKRQKLSNTSSPQKQEHNLNPRERLERQKSLISKSHEMRNRGSKKIVPTLGYPWATRIKNRIMLSKVYKPILKSNMDGDIVNDDQSLQTNSIHDKRSSAELYKSFVSIDGWQVERFAKVVTSTHGYNPNRFKRYRFIINSQGLAEV

SEQ ID NO: 55 - protein, 624 aa, Candida albicans
MSSLQLSKGALKQVFSKEGHDSVQIPMILQITNIKAFDVSPSDSKKFRILVNDGVYSTHGLIDESCSEYIKNNNCQRYAIVQVNAFSIFATSKHFFVIKNFEVLAPTSEKSPNNIIPIDTYFLEHPEENYLTVMKKSESRDRESPVPGVTPPLAQSTNSFKSEVGGGVAAQSKPAGTHRKVSPIETISPYQNNWTIKARVSYKGDLRTWSNSKGEGKVFGFNLLDESDEIKASAFNETAERAHKLLEEGKVYYISKARVAAARKKFNTLSHPYELTFDKDTEITECFDESDVPKLNFNFVKLDQVQNLEANAIIDVLGALKTVFPPFQITAKSTGKVFDRRNILVVDETGFGIELGLWNNTATDFNIEEGTVVAVKGCKVSDYDGRTLSLTQAGSIIPNPGTPESFKLKGWYDNIGIHESFKSLKIDNAGSGGDKISQRISINQALEEHSGSTEKPDYFSIKASVTFCKPENFAYPACPNLVQNADATRPAQVCNKKLVFQDNDGTWRCERCAKTYEEPTWRYVLSCSVTDSTGHMWVTLFNDQAEKLLGIDATELVKKKEQKSEVANQIMNNTLFKEFSLRVKAKQETYNDELKTRYSAAGINELDYASESQFLIKKLDQLLK

SEQ ID NO: 56 - protein, 854 aa, Saccharomyces cerevisiae
MWVVRYQNTLEDGSISFISCCLQAFKTYSIGRSSKNPLIIKNDKSISRQHITFKWEINNSSDLKHSSLCLVNKGKLTSLNKKFMKVGETFTINASDVLKSTIIELGTTPIRIEFEWINEVWNIPPHLTQFRTMLSEYGISTEISINDIPANLMISDYPKSEDNSIRELYALVSTIPMKKSRFLMELCNTLLPTSKTNLKFDEMWNDMISNPEYNVFDFDPNILLSKFMRLNNIRVLTTIKSEPRLSSLLRTFNINLFAFDNIDSLYKYVDSLEASTEYLILTTTDKKENGKILCTIKTMLTSIIDGTLSAVINMKGASSRTLDNGKFDQISEGMSTILKTSRAPEVEASPVVSKKRKLNRRRVLPLDSLDFFAGGLSTKTLSENRSLTDAKRLNCGAESKTVISSPNIAEADEKHAPFLQNALKPTEDIGKKSGHSSPGAIIVSSPNLGTVNTSEDSLDKSLQSHKLPQPSLPEVAGIGSQTISSNSADYETAAVNSMDDAEVTKNFRVNHHQNIEQPSKNIRKLSNYSREISSPLQENCKSPVKELSIKEKSGTPHAFVEAIQETKNREVKRVKSTIVELKDEELSEEAINQLKNLAIVEPSNNLLRKSFDSEGNKTSRTTEKWENSLMEPEWHKRKNFKTFVKVRPKSKAHKEEGKNNTQSSDFIRNAAFLITRNYVPLKKYSKKDTTTKWGTEENEDMFALTEMERFGSNTFMSDNINSNTIQKRSQALNRFTNEDSSNEGEEDSFSFSRCSGTAASVQPLKNKIFITDEDDLGDIDDKSDRLNHRENNRNLFVVKEMNLRPNLSEECSKQSRHSRSATSRSRGSFGASNNGDGDDDDDDGPKFTFKRRKG

SEQ ID NO: 57 - protein, 682 aa, Candida albicans
MPLVERIEPGPDTIRVLLTTDNHVGAFENDPIRGDDAWKTFDEITTIAKDKDVDMIIQGGDLFHINKPTKKSMYHVMKSLRSNCMGDRPCELELLSDPAQSLNNGFDEINYEDPNLNISIPVFAISGNHDDATGESLLSALDVLAVTGLINNFGKVKNTEAITVSPILLQKGQTKLALYGMSNVRDERLHRLFRDGGVKFQRPNIQTEDWFNLFVIHQNHAAHTYTSSIPESFLPNFLDFILWGHEHECIPYPVHNPETTFDVLQAGSSVATSLAEGEVADKKIFILNIKGKDYSIEPVELKTVRPFVLREIILSKTDLIPGAASKADVIAYLTDEVEKSIERANKQFSSQNISNSNRAITNSSNNATADPIEKPLPLIRLRVEYSGGYEIENVTRFSNRFVGKIANVNDVVQFYKKKTPTKSDNKLTRKTKYDVDLIEENLHHKKTTELELQDIIRDFLQQTQLSLVPETEMNHAVKKFVENDDKQALNQFINQEIKRETKMLLDIDIDENEFHGADEKHAKTAFKHVLSQLKNINGPINIDYEPEIEPTNNNTNKKSATTKKRTPKKNTTIPKKITTTATKKPKIDDIIISSDDSNDYGNDDDDDKEEEEEENEHDSGLRLFVTDTPDTNSTRRSKSNRSKRPTSYVEDESGILSDEDDYVPPSKSKGIFSRSFNNRKRK

SEQ ID NO: 58 - protein, 421 aa, Saccharomyces cerevisiae
MSQLTEFISCIPVVNEEQNEEDERGLCKIQIEDGAMLETLDENSLSGLRIEKMLVSEGTGIFSKSSFGINDLRIFTGENIDEESKKYVWYELLKMLTGHKVYIASLDEKVVFTKWTCRMQDDEVWKVVMELESSAIIRKIAELTLHPVKKGEIDLFEMADKLYKDICCVNDSYRNIKESDSSNRNRVEQLARERELLDKLLETRDERTRAMMVTLLNEKKKKIRELHEILRQNNIKLSDDDVLDSALINTEVQKPISELNSPGKRMKRRKTVVEPQNLQKKLKDTSRRRANRKISNQSVIKMEDDDFDDFQFFGLSKRPIITAKDKLSEKYDDITSFGDDTQSISFESDSSSDVQKHLVSLEDNGIQISAGRSDEDYGDISGSESETDASAGEKKSSNHSEQSGNDREPCLQTESETDIET

SEQ ID NO: 59 - protein, 342 aa, Saccharomyces cerevisiae
MDSELKGQQLSDAEWCVKKINGEGNCLLLFLPMSSPTTIVMIVLVSLERLVPYVFKLSQTQLSQQCQSQGFTDSISLNLIKLKLMDILQAPQEINQIGLVDSNLVFSFDVSADITVSINSVPSHVTKDMFYMILQSLCMLLLKLVNLSTQYHYVQRDILNEKQKCLDFLLISLRDLDGGSKVISQWAPENSKNYESLQQCTDDDIIKKLLHKGKFQHQEFLADSLKTLLSLRNKFQDVSRFEESGELNKKERVRFPAVNHFYNDDFELQADPTNEARPNSRGKIKPKTDFKPKSRESSTSSQLRLENFSESEATPEKTKSSSSLVEEYPQKKRKFGKVRIKN

SEQ ID NO: 60 - protein, 636 aa, Saccharomyces cerevisiae
MTNALLSIAVLLFSMLSLAQAETHTFNWTTGWDYRNVDGLKSRPVITCNGQFPWPDITVNKGDRVQIYLTNGMNNTNTSMHFHGLFQNGTASMDGVPFLTQCPIAPGSTMLYNFTVDYNVGTYWYHSHTDGQYEDGMKGLFIIKDDSFPYDYDEELSLSLSEWYHDLVTDLTKSFMSVYNPTGAEPIPQNLIVNNTMNLTWEVQPDTTYLLRIVNVGGFVSQYFWIEDHEMTVVEIDGITTEKNVTDMLYITVAQRYTVLVHTKNDTDKNFAIMQKFDDTMLDVIPSDLQLNATSYMVYNKTAALPTQNYVDSIDNFLDDFYLQPYEKEAIYGEPDHVITVDVVMDNLKNGVNYAFFNNITYTAPKVPTLMTVLSSGDQANNSEIYGSNTHTFILEKDEIVEIVLNNQDTGTHPFHLHGHAFQTIQRDRTYDDALGEVPHSFDPDNHPAFPEYPMRRDTLYVRPQSNFVIRFKADNPGVWFFHCHIEWHLLQGLGLVLVEDPFGIQDAHSQQLSENHLEVCQSCSVATEGNAAANTLDLTDLTGENVQHAFIPTGFTKKGIIAMTFSCFAGILGIITIAIYGMMDMEDATEKVIRDLHVDPEVLLNEVDENEERQVNEDRHSTEKHQFLTKAKRFF

SEQ ID NO: 61 - protein, 4 aa, Artificial Sequence
  source: /note=“Description of Artificial Sequence Synthetic peptide”
EALA

SEQ ID NO: 62 - protein, 29 aa, Artificial Sequence
  source: /note=“Description of Artificial Sequence Synthetic peptide”
AALEALAEALEALAEALEALAEAAAAGGC

SEQ ID NO: 63 - protein, 30 aa, Artificial Sequence
  source: /note=“Description of Artificial Sequence Synthetic polypeptide”
AALAEALAEALAEALAEALAEALAAAAGGC

SEQ ID NO: 64 - protein, 15 aa, Artificial Sequence
  source: /note=“Description of Artificial Sequence Synthetic peptide”
ALEALAEALEALAEA

SEQ ID NO: 65 - protein, 16 aa, Artificial Sequence
  source: /note=“Description of Artificial Sequence Synthetic peptide”
AAVALLPAVLLALLAP

SEQ ID NO: 66 - protein, 11 aa, Artificial Sequence
  source: /note=“Description of Artificial Sequence Synthetic peptide”
AALLPVLLAAP

SEQ ID NO: 67 - protein, 13 aa, Human immunodeficiency virus
GRKKRRQRRRPPQ

SEQ ID NO: 68 - protein, 16 aa, Drosophila sp.
RQIKIWFQNRRMKWKK